(19) United States
(12) Patent Application Publication
(10) Pub. No.: US 2022/0372066 A1
(43) Pub. Date: Nov. 24, 2022

(54) HUMAN SQUALAMINE DERIVATIVES (ENT-06), RELATED COMPOSITIONS COMPRISING THE SAME, AND METHOD OF USING THE SAME

(71) Applicant: Enterin, Inc., Philadelphia, PA (US)

(72) Inventors: Denise Barbut, Philadelphia, PA (US); Michael Zasloff, Philadelphia, PA (US)

(73) Assignee: Enterin, Inc., Philadelphia, PA (US)

(21) Appl. No.: 17/631,887

(22) PCT Filed: Jul. 31, 2020
(86) PCT No.: PCT/US2020/044392

§ 371 Date: Jan. 31, 2022

Related U.S. Application Data
(60) Provisional application No. 62/882,318, filed on Aug. 2, 2019.
(60) Provisional application No. 63/036,828, filed on Jun. 9, 2020.

(51) Int. Cl. C07J 9/00; C07J 71/00; A61P 25/18; A61P 25/28

(52) U.S. Cl. CPC C07J 9/005; C07J 71/0026; A61P 25/18; A61P 25/28

(57) ABSTRACT

The present application relates generally to novel aminosterol compounds, compositions comprising the same, and methods of making and using the novel aminosterol compounds and compositions.

### CROSS REFERENCE TO RELATED APPLICATIONS

This application claims the priority benefits under 35 USC § 119 to U.S. provisional Application No. 62/882,318, filed Aug. 2, 2019, and U.S. provisional Application No. 63/036,828, filed Jun. 9, 2020, the entire contents each of which is incorporated herein by reference in its entirety.

### FIELD

The present application relates generally to a novel compounds for the treatment of disease.

### BACKGROUND

Aminosterols are amino derivatives of a sterol. Squalamine is the most abundant member of a larger aminosterol family comprising at least 12 related compounds (Rao et al., 2000). Exemplary aminosterols include squalamine and ENT-02 (also known as aminosterol 1436, trodusquemine and MSI-1436). These aminosterols were discovered by Michael Zasloff in the spiny dogfish shark Squalus acanthias (U.S. Pat. No. 5,192,756) and they exhibit diverse pharmacological activity in mammalian systems. Aminosterol 1436 exhibits pharmacology in vertebrates causing weight loss and adipose tissue mobilization (Zasloff et al., 2001). Squalamine has antiviral, antibiotic, antifungal, and anticancer activity, and inhibits aggregation of the α-synuclein protein characteristic in Parkinson's disease (Zasloff et al., 2011; Moore et al., 1993; Perni et al., 2019; Ahima et al., 2002; Brunel et al., 2005).

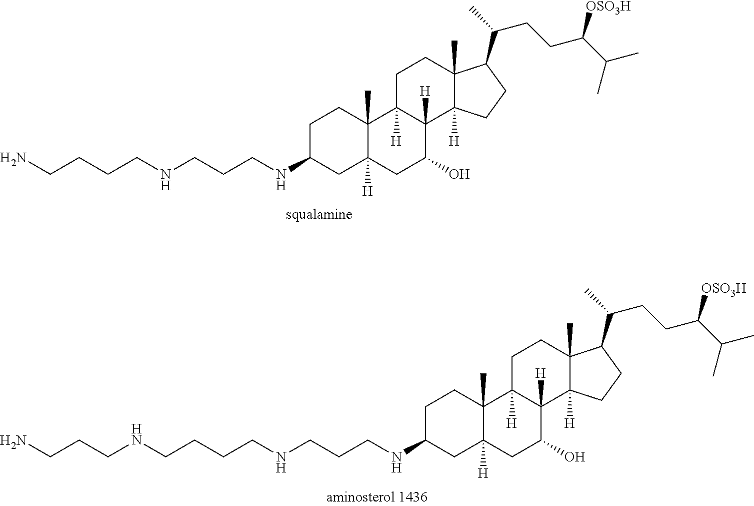

Several clinical trials have been conducted relating to the use of squalamine, including: (a) ClinicalTrials.gov Identifier NCT01769183 for “Squalamine for the Treatment in Proliferative Diabetic Retinopathy,” by Elman Retina Group (6 participants; study completed August 2014); (b) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017; (c) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014); (d) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015); (e) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated); (f) ClinicalTrials.gov Identifier NCT00094120 for “MSI-1256F (Squalamine Lactate) in Combination With Verteporfin in Patients With “Wet” Age-Related Macular Degeneration (AMD),” by Genaera Corporation (60 participants; study completed February 2007); and (g) ClinicalTrials.gov Identifier NCT00089830 for “A Safety and Efficacy Study of MSI-1256F (Squalamine Lactate) To Treat “Wet” Age-Related Macular Degeneration,” by Genaera Corporation (120 participants; study completed May 2007).

There is a need in the art for novel aminosterol compounds and methods of using the same. The present disclosure satisfies these needs.

### SUMMARY

In one aspect, an aminosterol compound having the formula:

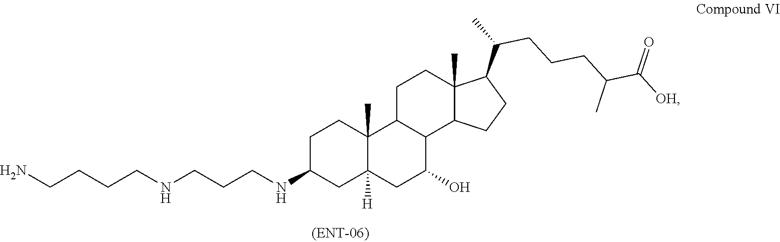 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]

is provided, or a pharmaceutically acceptable salt, solvate, prodrug, or derivative thereof.

In one embodiment, the aminosterol has the formula:

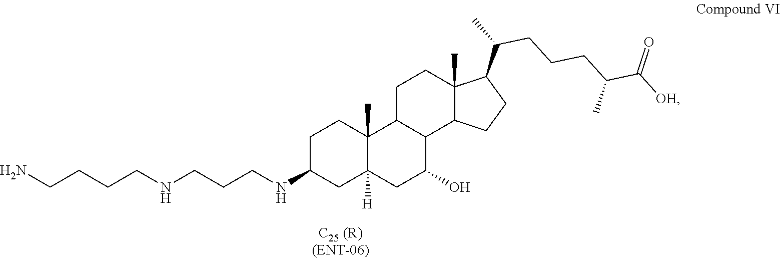 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

or a pharmaceutically acceptable salt, solvate, prodrug, or derivative thereof.

In another aspect, an aminosterol compound having the formula:

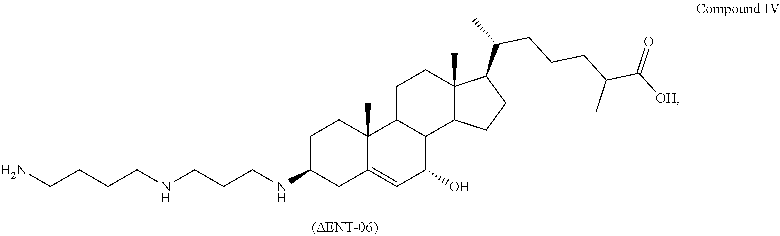 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

is provided, or a pharmaceutically acceptable salt, solvate, prodrug, or derivative thereof.

In one embodiment, the aminosterol compound has the formula:

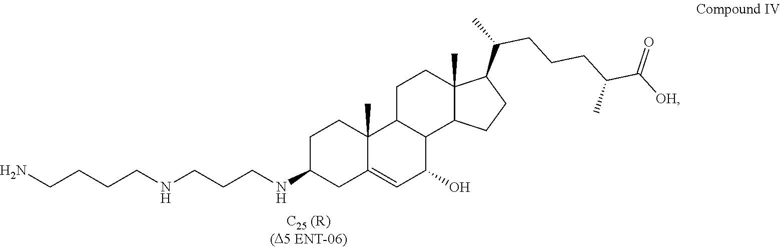 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]

or a pharmaceutically acceptable salt, solvate, prodrug, or derivative thereof.

In one aspect, an aminosterol compound having the formula:

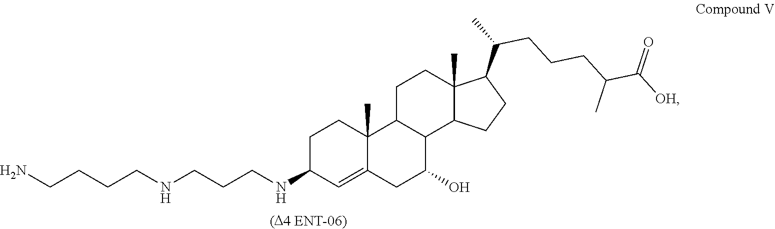 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, or a pharmaceutically acceptable salt, solvate, prodrug, or derivative thereof.

In one embodiment, the aminosterol compound has the formula:

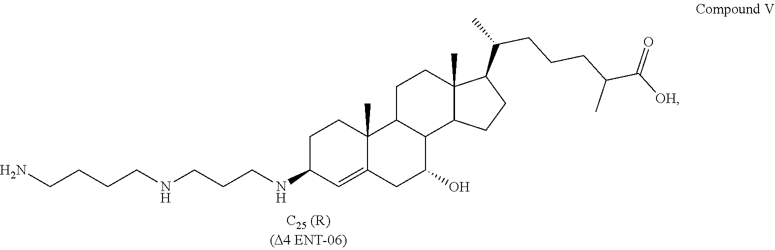 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

or a pharmaceutically acceptable salt, solvate, prodrug, or derivative thereof.

In another aspect, an aminosterol compound having the formula:

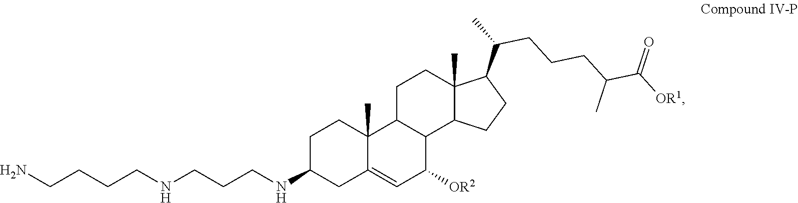

is provided wherein: R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; provided that at least one of R¹ and R² is not H, or a pharmaceutically acceptable salt, solvate, prodrug, or derivative thereof.

In one embodiment, the aminosterol compound has the formula:

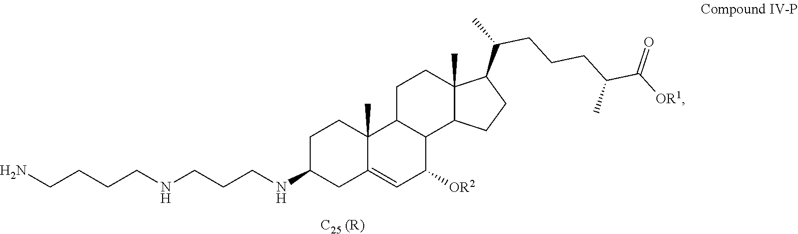

or a pharmaceutically acceptable salt, solvate, prodrug, or derivative thereof.

In another aspect, an aminosterol compound having the formula:

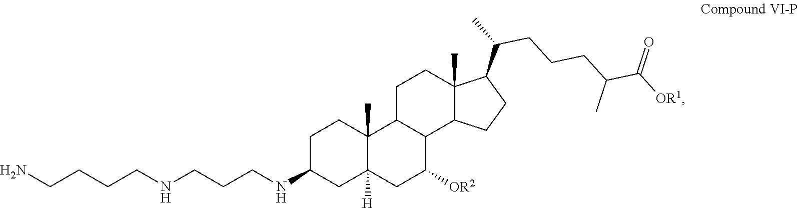

is provided, wherein: R¹ is H, optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; and R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; provided that at least one of R¹ and R² is not H, or a pharmaceutically acceptable salt, solvate, prodrug, or derivative thereof.

In some embodiments, the aminosterol has the formula:

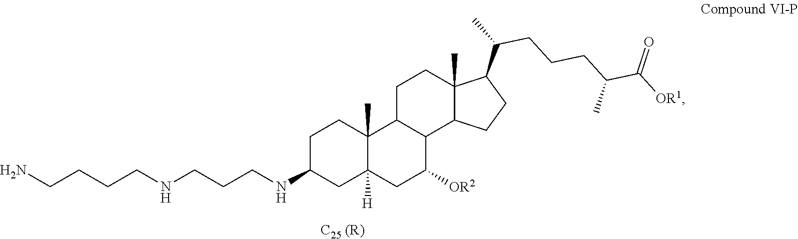

or a pharmaceutically acceptable salt, solvate, prodrug, or derivative thereof.

In another aspect, an aminosterol compound having the formula:

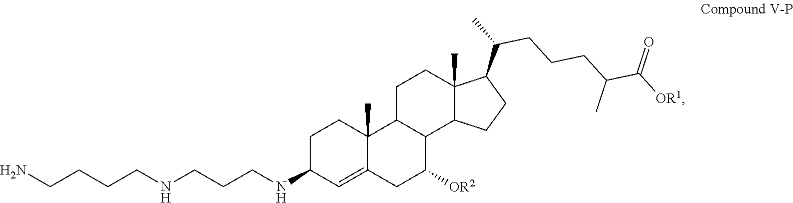

is provided wherein: R is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; provided that at least one of R¹ and R² is not H, or a pharmaceutically acceptable salt, solvate, prodrug, or derivative thereof.

In one embodiment, the aminosterol compound has the formula:

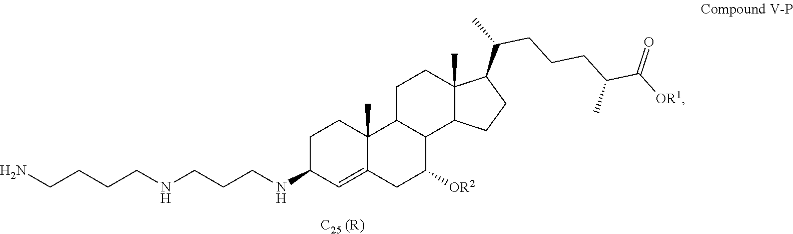

or a pharmaceutically acceptable salt, solvate, prodrug, or derivative thereof.

In some embodiments, the aminosterol is formulated as a pharmaceutically acceptable salt. In some embodiments, the pharmaceutically acceptable salt is a phosphate salt.

In one aspect, a composition comprising an aminosterol compound according to any embodiment herein is provided, the composition comprising at least one pharmaceutically acceptable carrier or excipient. In some embodiments, the composition comprises one or more of the following: (a) an aqueous carrier; (b) a buffer; (c) a sugar; and/or (d) a polyol compound. In some embodiments, the composition comprises at least one additional active agent.

In some embodiments, the composition is formulated (a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

In some embodiments, the composition is formulated for oral administration. In some embodiments, the composition is formulated as an oral tablet or capsule. In some embodiments, the composition is formulated for intranasal administration.

In one aspect a method of treating a subject in need is provided, the subject having a condition susceptible to treatment with an aminosterol, the method comprising administering to the subject the composition according to any embodiment herein. In some embodiments, the condition is correlated with abnormal alpha-synuclein pathology and/or dopaminergic dysfunction.

In another aspect, a method of treating, preventing, and/or slowing the onset or progression of a condition or disorder, or a related symptom, correlated with abnormal alpha-synuclein pathology and/or dopaminergic dysfunction, in a subject in need, is provided, the method comprising administering a therapeutically effective amount of a composition according to any embodiment herein.

In some embodiments (a) the symptom is selected from the group consisting of constipation, hallucinations, cognitive impairment, and inflammation; (b) the symptom is correlated with a synucleopathy, a neurodegenerative disease, a neurological disease or disorder, a psychological and/or behavior disorder, or a cerebral or general ischemic disorder or condition; or (c) the condition or disorder is a synucleopathy, neurodegenerative disease, or neurological disease or disorder; (d) the condition or disorder is a psychological and/or behavior disorder; or (e) the condition or disorder is a cerebral or general ischemic disorder or condition.

In some embodiments, (a) the synucleopathy, neurodegenerative disease, or neurological disease or disorder is selected from the group consisting of Parkinson's disease, Alzheimer's disease, schizophrenia, multiple system atrophy, Lewy body dementia, dementia with Lewy bodies, Huntington's Disease, Multiple Sclerosis, Amyotorphic Lateral Sclerosis, Friedreich's ataxia, vascular dementia, spinal muscular atrophy, supranuclear palsy, progressive nuclear palsy, frontotemporal dementia, progressive nuclear palsy, Guadeloupian Parkinsonism, spinocerebellar ataxia, parkinsonism, traumatic brain injury, degenerative processes associated with aging, and dementia of aging; (b) the psychological or behavior disorder is selected from the group consisting of depression, autism, autism spectrum disorder, down syndrome, Gaucher's disease, Krabbe's disease, lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, and sleep disorders such as REM sleep behavior disorder (RBD), sleep fragmentation, REM behavior disorder, circadian rhythm dysfunction, sleep apnea, and cognitive impairment; or (c) the cerebral or general ischemic disorder or condition is selected from the group consisting of microangiopathy, intrapartum, cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure, and pulmonary edema.

In another aspect, a method of treating, preventing, and/or slowing the onset or progression a cerebral or general ischemic disorder and/or a related symptom, correlated with abnormal alpha-synuclein pathology and/or dopaminergic dysfunction, in a subject in need, is provided, the method comprising administering a therapeutically effective amount of a composition according to any embodiment herein.

In one embodiment, the cerebral or general ischemic disorder and/or a related symptom is selected from the group consisting of microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, low blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects (CCDs) and/or a related symptom, and pulmonary edema.

In one aspect, a method of inhibiting protein tyrosine phosphatase 1B (PTP1B) in a subject is provided, the method comprising administering to the subject a therapeutically effective amount of a composition according to any embodiment herein.

In another aspect, a method of increasing transcription in the gut of a subject is provided, the method comprising administering to the subject a therapeutically effective amount of an aminosterol compound of any embodiment herein, or a pharmaceutically acceptable salt, solvate, prodrug, or derivative thereof.

In some embodiments, the increase in transcription is for one or more genes selected from caspase 14, collagen type XVII alpha 1, corneodesmosin, cornifelin, cystatin E/M, dermokine, desmocollin 1, desmoglein 1 beta, filaggrin, gap junction protein beta 4, gap junction protein beta 6, H19 imprinted maternally expressed transcript, hornerin, kallikrein related-peptidase 7 chymotryptic stratum, keratin 1, keratin 10, keratinocyte differentiation associated protein, keratinocyte expressed proline-rich, late cornified envelope 1A1, late cornified envelope 1A2, late cornified envelope 1B, late cornified envelope 1C, late cornified envelope 1E, late cornified envelope 1F, late cornified envelope 1G, late cornified envelope 1H, late cornified envelope 1I, late cornified envelope 1J, late cornified envelope 1L, late cornified envelope 1M, late cornified envelope 3C, late cornified envelope 3E, late cornified envelope 3F, lectin galactose binding soluble 7, loricrin, sciellin, myoglobin, myosin binding protein C slow-type, myosin heavy polypeptide 1 skeletal muscle, myosin heavy polypeptide 8 skeletal muscle, myosin light chain phosphorylatable fast ske, myosin light polypeptide 3, myozenin 1, myozenin 2, and titin-cap.

In some embodiments, the method further comprises administering to the subject one or more non-aminosterol compounds that upregulate or down regulate one or more genes selected from caspase 14, collagen type XVII alpha 1, corneodesmosin, cornifelin, cystatin E/M, dermokine, desmocollin 1, desmoglein 1 beta, filaggrin, gap junction protein beta 4, gap junction protein beta 6, H19 imprinted maternally expressed transcript, hornerin, kallikrein related-peptidase 7 chymotryptic stratum, keratin 1, keratin 10, keratinocyte differentiation associated protein, keratinocyte expressed proline-rich, late cornified envelope 1A1, late cornified envelope 1A2, late cornified envelope 1B, late cornified envelope 1C, late cornified envelope 1E, late cornified envelope 1F, late cornified envelope 1G, late cornified envelope 1H, late cornified envelope 1I, late cornified envelope 1J, late cornified envelope 1L, late cornified envelope 1M, late cornified envelope 3C, late cornified envelope 3E, late cornified envelope 3F, lectin galactose binding soluble 7, loricrin, sciellin, myoglobin, myosin binding protein C slow-type, myosin heavy polypeptide 1 skeletal muscle, myosin heavy polypeptide 8 skeletal muscle, myosin light chain phosphorylatable fast ske, myosin light polypeptide 3, myozenin 1, myozenin 2, and titin-cap. The non-aminosterol compound may be any compound known in the art to regulate any of the aforementioned genes.

In some embodiments, the increase in transcription is selected from about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, about 250% to about 300%, about 300% to about 350%, about 350% to about 400%, about 400% to about 450%, about 500% to about 600%, about 600% to about 700%, about 700% to about 800%, about 800% to about 900%, about 900% to about 1000%, or about 1000% to about 1500%.

In some embodiments, the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In some embodiments, the method of administration is nasal administration, oral administration, or a combination thereof.

In some embodiments, the therapeutically effective amount of the aminosterol compound or a pharmaceutically acceptable salt, solvate, prodrug, or derivative thereof comprises: (a) about 0.1 to about 20 mg/kg body weight of the subject; (b) about 0.1 to about 15 mg/kg body weight of the subject; (c) about 0.1 to about 10 mg/kg body weight of the subject; (d) about 0.1 to about 5 mg/kg body weight of the subject; or (e) about 0.1 to about 2.5 mg/kg body weight of the subject.

In some embodiments, the therapeutically effective amount of the aminosterol compound or a pharmaceutically acceptable salt, solvate, prodrug, or derivative thereof comprises: (a) about 0.001 to about 500 mg/day; (b) about 0.001 to about 250 mg/day; (c) about 0.001 to about 125 mg/day; (d) about 0.001 to about 50 mg/day; (e) about 0.001 to about 25 mg/day; (f) about 0.001 to about 10 mg/day; (g) about 0.001 to about 6 mg/day; (h) about 0.001 to about 4 mg/day; or (i) about 0.001 to about 2 mg/day.

In some embodiments, the method of administration comprises oral administration and the therapeutically effective amount of the aminosterol compound or a pharmaceutically acceptable salt, solvate, prodrug, or derivative thereof comprises: (a) about 1 to about 300 mg/day; or (b) about 25 to about 500 mg/day; or (c) about 1 to about 500 mg/day.

In some embodiments, the aminosterol compound or a pharmaceutically acceptable salt, solvate, prodrug, or derivative thereof is administered in combination with at least one additional active agent to achieve either an additive or synergistic effect. In some embodiments, the additional active agent is administered via a method selected from the group consisting of (a) concomitantly; (b) as an admixture; (c) separately and simultaneously or concurrently; and (d) separately and sequentially. In some embodiments, the additional active agent is a second aminosterol having a different structure from the aminosterol administered in any embodiment herein.

In some embodiments, administration of the composition comprises administration on an empty stomach, optionally within two hours of the subject waking. In some embodiments, no food is consumed by the subject after about 60 to about 90 minutes from administration of the composition.

In some embodiments, the aminosterol, or a pharmaceutically acceptable salt, solvate, prodrug, or derivative thereof, is of pharmaceutically acceptable grade. In some embodiments, a phosphate salt of the aminosterol is administered. In some embodiments, the subject is a human.

In some embodiments, the method further comprises (a) determining a dosage of the aminosterol or a pharmaceutically acceptable salt, solvate, prodrug, or derivative for the subject, wherein the aminosterol dosage is determined based on the effectiveness of the aminosterol dosage in improving or resolving a symptom being evaluated, (b) followed by administering a composition comprising the dosage of the aminosterol to the subject for a period of time, wherein the method comprises: (i) identifying a symptom to be evaluated, wherein the symptom is susceptible to treatment with an aminosterol; (ii) identifying a starting dosage of an aminosterol thereof for the subject; (iii) administering an escalating dosage of the aminosterol to the subject over a period of time until an effective dosage for the symptom being evaluated is identified, wherein the effective dosage is the aminosterol dosage where improvement or resolution of the symptom is observed, and fixing the aminosterol dosage at that level for that particular symptom in that particular subject. In some embodiments, improvement or resolution of the symptom is measured using a clinically recognized scale or tool.

In some embodiments, the composition is administered orally and: (a) the starting aminosterol dosage ranges from about 10 mg up to about 150 mg/day; (b) the dosage of the aminosterol for the subject following escalation is fixed at a range of from about 25 mg up to about 500 mg/day; and/or (c) the dosage of the aminosterol or a salt or derivative thereof is escalated in about 25 mg increments.

In some embodiments, the composition is administered intranasally and: (a) the starting aminosterol dosage ranges from about 0.001 mg to about 3 mg/day; (b) the dosage of the aminosterol for the subject following escalation is fixed at a range of from about 0.001 mg up to about 6 mg/day; (c) the dosage of the aminosterol for the subject following escalation is a dosage which is subtherapeutic when given orally or by injection; and/or (c) the dosage of the aminosterol is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In some embodiments, the dosage of the aminosterol is escalated every about 3 to about 5 days. In some embodiments, the starting aminosterol dosage is higher if the symptom being evaluated is severe.

In some embodiments, the symptom is correlated with abnormal alpha-synuclein pathology and/or dopaminergic dysfunction. In some embodiments, the symptom to be evaluated is selected from the group consisting of: (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (1) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

In some embodiments, the symptom to be evaluated is constipation, and wherein: (a) the fixed escalated aminosterol dosage for constipation is defined as the aminosterol dosage that results in a complete spontaneous bowel movement (CSBM) within 24 hours of dosing on at least 2 of 3 days at a given dosage; (b) if average complete spontaneous bowel movement (CSBM) or average spontaneous bowel movement (SBM) is greater than or equal to 1 per week, then the starting aminosterol dosage prior to escalation is 75 mg/day; and/or (c) if average CSBM or SBM is less than 1 per week, then the starting aminosterol dosage prior to escalation is 150 mg/day.

In one aspect, a method of producing an aminosterol of formula:

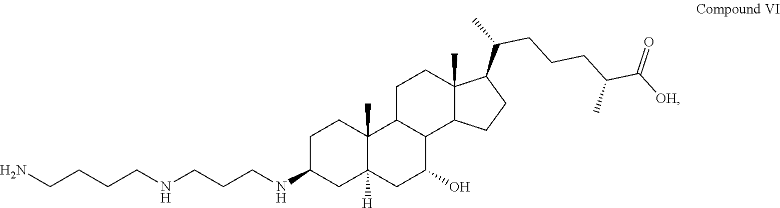 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

is provided, the method comprising stimulating the addition of spermidine to Compound Ia:

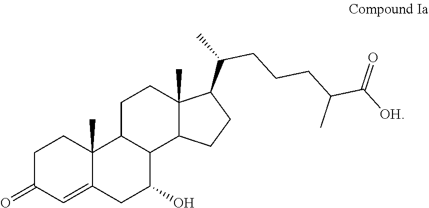 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the aminosterol is produced in vivo in a subject; or (b) the aminosterol is produced in vitro.

In another aspect, a method of suppressing the formation of an aminosterol of formula:

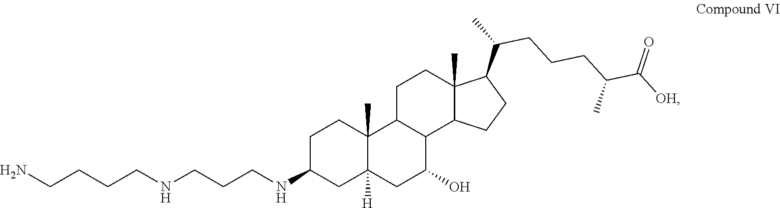 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

is provided, the method comprising suppressing the addition of spermidine to Compound Ia:

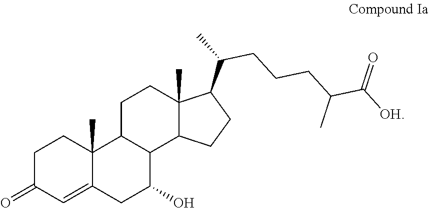 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the addition of spermidine to Compound Ia is suppressed in vivo in a subject; or (b) the addition of spermidine to Compound Ia is suppressed in vitro.

In some embodiments, Compound Ia has the formula:

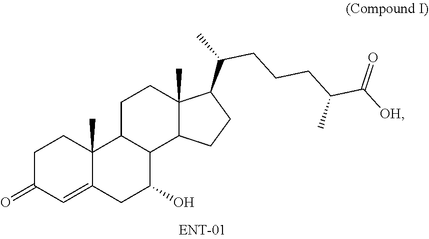 [SMILES: C[C@H](CCC[C@@H](C)[C@H]1CCC2C3C(CC[C@@]21C)[C@@]1(C)CCC(=O)C=C1C[C@H]3O)C(=O)O]

and Compound VI has the formula:

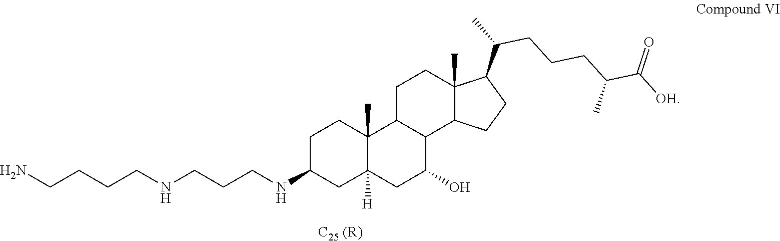 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

In another aspect, a method of producing an aminosterol of formula:

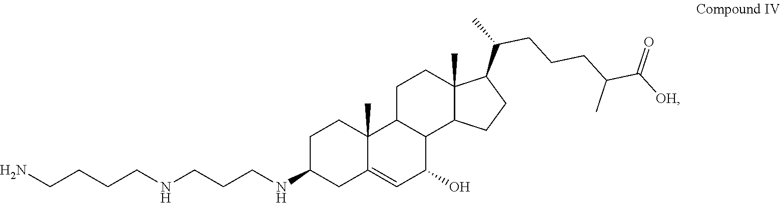 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

is provided, the method comprising stimulating the addition of spermidine to Compound Ia:

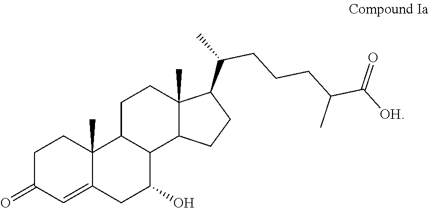 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the aminosterol is produced in vivo in a subject; or (b) the aminosterol is produced in vitro.

In another aspect, a method of suppressing the formation of an aminosterol of formula:

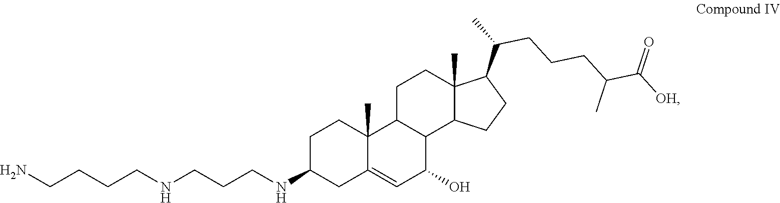 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

is provided, the method comprising suppressing the addition of spermidine to Compound Ia:

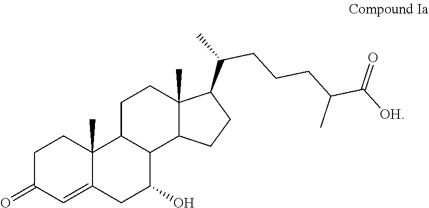 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the addition of spermidine to Compound Ia is suppressed in vivo in a subject; or (b) the addition of spermidine to Compound Ia is suppressed in vitro.

In some embodiments, Compound Ia has the formula:

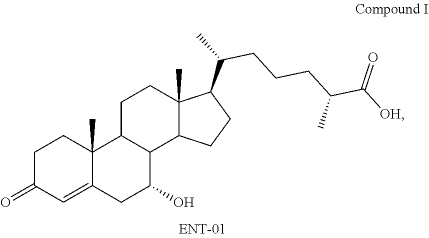 [SMILES: C[C@H](CCC[C@@H](C)[C@H]1CCC2C3C(CC[C@@]21C)[C@@]1(C)CCC(=O)C=C1C[C@H]3O)C(=O)O]

and Compound IV has the formula:

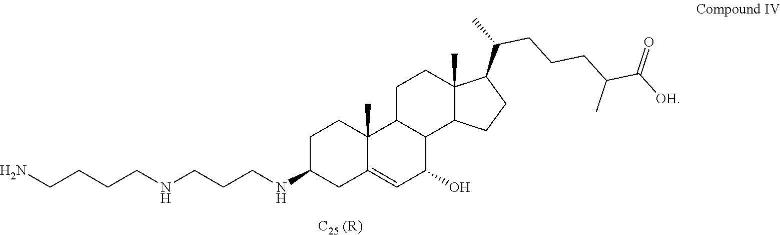 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

In another aspect, a method of producing an aminosterol of formula:

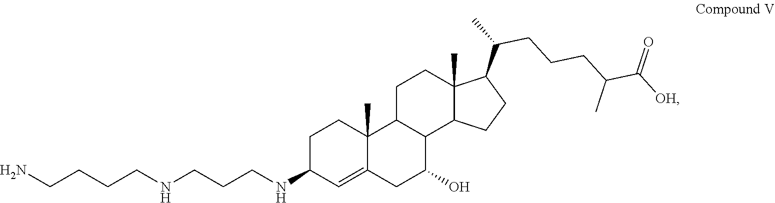 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, the method comprising stimulating the addition of spermidine to Compound Ia:

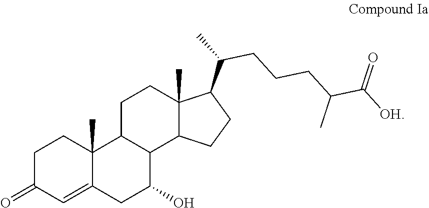 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the aminosterol is produced in vivo in a subject; or (b) the aminosterol is produced in vitro.

In another aspect, a method of suppressing the formation of an aminosterol of formula:

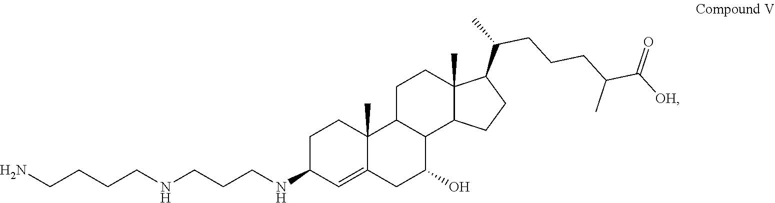 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, the method comprising suppressing the addition of spermidine to Compound Ia:

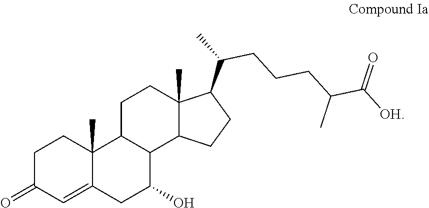 [SMILES: CC(CCC[C@@H](C)[C@H]1CCC2C3C(CC[C@@]21C)[C@@]1(C)CCC(=O)C=C1C[C@H]3O)C(=O)O]

In some embodiments, (a) the addition of spermidine to Compound Ia is suppressed in vivo in a subject; or (b) the addition of spermidine to Compound Ia is suppressed in vitro.

In some embodiments, Compound Ia has the formula:

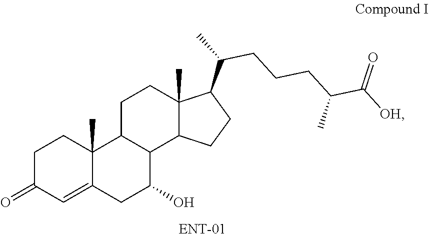 [SMILES: C[C@H](CCC[C@@H](C)[C@H]1CCC2C3C(CC[C@@]21C)[C@@]1(C)CCC(=O)C=C1C[C@H]3O)C(=O)O]

and Compound V has the formula:

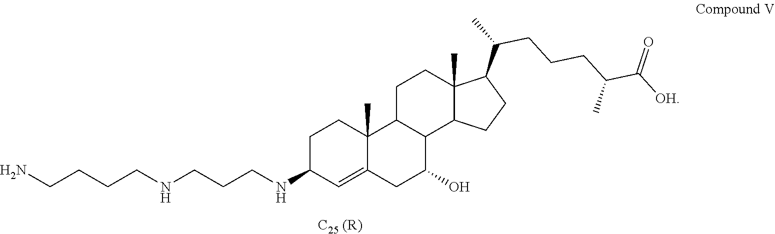 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1: Images of a mucosal layer of the stomach (in a mouse) showing a reduced mucosal layer in the 78 week old mouse stomach (FIG. 1B) vs. the younger 20 week old mouse stomach (FIG. 1A).

FIG. 2: Transcriptional changes in response to ENT-01 in the stomach of old and young mice: Sina plot showing the magnitude of fold change (log 2 fold change) of significantly differentially expressed genes between ENT-01-treated and control samples from the stomach tissues of old and young mice. Significantly up-regulated genes (fold change>4) were represented as red dots and significantly down-regulated genes (fold change<4) as blue dots.

FIG. 3: Ageing associated gene expression changes reversed by ENT-01 treatment: Sina plots showing the magnitude of fold change (log 2 fold change) of significantly differentially expressed genes between contrasts ‘Old vs young (control)’ and either ‘ENT-01 vs control (old)’ (FIG. 3A) or ‘ENT-01 vs control (young)’ (FIG. 3B). Green dots represent genes that were significantly down-regulated in ageing and significantly up-regulated upon ENT-01 treatment. Orange dots represented genes that were significantly up-regulated in ageing and significantly down-regulated upon ENT-01 treatment. Grey dots represented genes that were significantly differentially expressed in a given contrast, but were not altered in the opposite direction in the other contrast.

FIG. 4: Heatmaps of overlaps between contrasts: A plot showing the number of overlapping selected genes between the contrasts performed. Note that the numbers on the diagonal represent the total number of selected genes found for each contrast. The colours of the squares represent the Jaccard index (the intersection over the union) for the contrasts on the x-axis with those on the y-axis. FIG. 4A: Heatmap of overlaps of up- and down-regulated (y-axis) vs. up- and down-regulated (x-axis) selected genes for each contrast. FIG. 4B: Heatmap of overlaps of up-regulated (y-axis) vs. up-regulated (x-axis) selected genes for each contrast. FIG. 4C: Heatmap of overlaps of down-regulated (y-axis) vs. down-regulated (x-axis) selected genes for each contrast. FIG. 4D: Heatmap of overlaps of up-regulated (y-axis) vs. down-regulated (x-axis) selected genes for each contrast.

FIG. 5: Venn diagram of transcripts down-regulated in ageing and up-regulated by ENT-01 or ENT-06: A plot showing the numbers of overlapping and non-overlapping differentially expressed genes between the two sets of transcripts that were down-regulated in old versus young mice and up-regulated upon treatment compared to control. Numbers of features are shown from treatment ENT-01 (left) and ENT-06 (right).

FIG. 6: Scatter plot comparing significant genes in ENT-01 vs control (young) against ENT-06 vs untreated (young). Genes are represented by points. The colour of the point indicates which set the gene is assigned to. For each gene the log 2(fold change) in the ENT-01 vs control (young) contrast (y-axis) and the log 2(fold change) in the ENT-06 vs untreated (young) contrast (x-axis) are shown.

FIG. 7: Upset plot of significant genes: A plot showing the interaction between sets of up- and down-regulated genes. The leftmost barchart shows the size of each set used as input. The top barchart shows the exclusive size of each set (i.e., each gene is only counted once in this barchart). The dot-plot in the centre shows the sets interacting in each case.

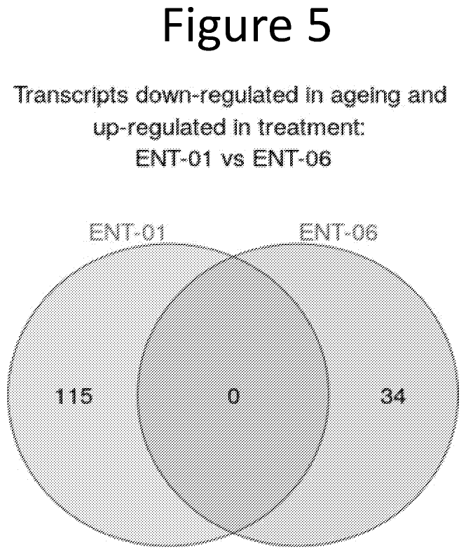

FIG. 8: Venn diagrams of significant genes in ENT-01 vs control (young) against ENT-06 vs untreated (young): Venn diagrams of up- and down-regulated genes. FIG. 8A: Venn diagram of overlapping genes in ENT-01 vs control (young) against ENT-06 vs untreated (young)—all vs all. FIG. 8B: Venn diagram of overlapping genes in ENT-01 vs control (young) against ENT-06 vs untreated (young) up vs up FIG. 8C: Venn diagram of overlapping genes in ENT-01 vs control (young) against ENT-06 vs untreated (young)—down vs down. FIG. 8D: Venn diagram of overlapping genes in ENT-01 vs control (young) against ENT-06 vs untreated (young)—up vs down. FIG. 8E: Venn diagram of overlapping genes in ENT-01 vs control (young) against ENT-06 vs untreated (young) down vs up.

FIG. 9: Scatter plot comparing significant genes in ENT-01 vs control (old) against ENT-06 vs untreated (old). Genes are represented by points. The colour of the point indicates which set the gene is assigned to. For each gene the log 2(fold change) in the ENT-01 vs control (old) contrast (y-axis) and the log 2(fold change) in the ENT-06 vs untreated (old) contrast (x-axis) are shown.

FIG. 10: Upset plot of significant genes: A plot showing the interaction between sets of up- and down-regulated genes. The leftmost barchart shows the size of each set used as input. The top barchart shows the exclusive size of each set (i.e., each gene is only counted once in this barchart). The dot-plot in the centre shows the sets interacting in each case.

FIG. 11: Venn diagrams of significant genes in ENT-01 vs control (old) against ENT-06 vs untreated (old): Venn diagrams of up- and down-regulated genes. Each plot considers a different interaction of sets; either ignoring direction of perturbation, considering only up-regulated genes, considering only down-regulated genes, or examining the overlap between those genes up-regulated in one contrast and those genes down-regulated in another. FIG. 11A: Venn diagram of overlapping genes in ENT-01 vs control (old) against ENT-06 vs untreated (old)—all vs all. FIG. 11B: Venn diagram of overlapping genes in ENT-01 vs control (old) against ENT-06 vs untreated (old)—up vs up. FIG. 11C: Venn diagram of overlapping genes in ENT-01 vs control (old) against ENT-06 vs untreated (old)—down vs down. FIG. 11D: Venn diagram of overlapping genes in ENT-01 vs control (old) against ENT-06 vs untreated (old)—up vs down.

FIG. 11E: Venn diagram of overlapping genes in ENT-01 vs control (old)—against ENT-06 vs untreated (old)—down vs up.

FIG. 12: Scatter plot comparing significant genes in Old vs young (ENT-01) against Old vs young (ENT-06): Genes are represented by points. The colour of the point indicates which set the gene is assigned to. For each gene the log 2(fold change) in the Old vs young (ENT-01) contrast (y-axis) and the log 2(fold change) in the Old vs young (ENT-06) contrast (x-axis) are shown.

FIG. 13: Upset plot of significant genes: A plot showing the interaction between sets of up and down-regulated genes. The leftmost barchart shows the size of each set used as input. The top barchart shows the exclusive size of each set (i.e., each gene is only counted once in this barchart). The dot-plot in the centre shows the sets interacting in each case.

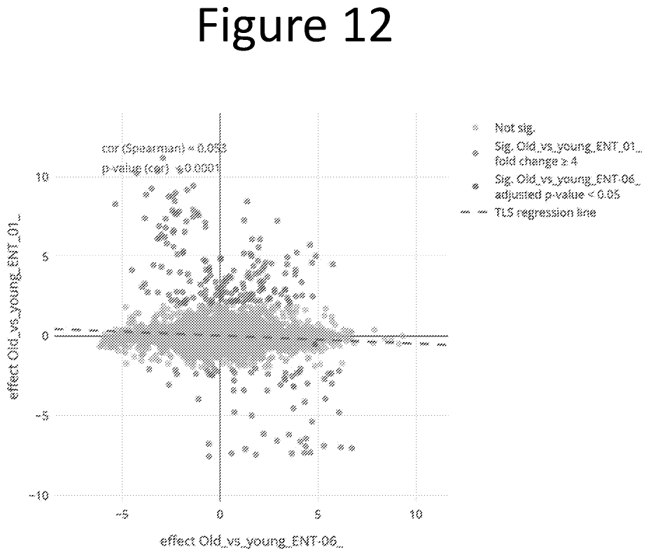

FIG. 14: Venn diagrams of significant genes in Old vs young (ENT-01) against Old vs young (ENT-06): Venn diagrams of up- and down-regulated genes. Each plot considers a different interaction of sets; either ignoring direction of perturbation, considering only up-regulated genes, considering only down-regulated genes, or examining the overlap between those genes up-regulated in one contrast and those genes down-regulated in another. FIG. 14A: Venn diagram of overlapping genes in Old vs young (ENT-01) against Old vs young (ENT-06)—all vs all. FIG. 14B: Venn diagram of overlapping genes in Old vs young (ENT-01) against Old vs young (ENT-06)—up vs up. FIG. 14C: Venn diagram of overlapping genes in Old vs young (ENT-01) against Old vs young (ENT-06) down vs down FIG. 14D: Venn diagram of overlapping genes in Old vs young (ENT-01) against Old vs young (ENT-06)—up vs down.

FIG. 14E: Venn diagram of overlapping genes in Old vs young (ENT-01) against Old vs young (ENT-6)—down vs up.

### DETAILED DESCRIPTION

### I. Overview

The pharmacological activities of aminosterols require a highly specific chemical structure, suggesting that the shark molecules are utilized by physiological circuits that exist to accommodate analogous compounds made by mammals. To date, no such compounds have been discovered nor even hypothesized to exist. This disclosure provides such compounds, modified versions thereof, and methods for their use. These methods include treatment and prevention of infection by a micororgnaism, including but not limited to a virus such as a coronavirus. Thus, the present technology relates to derivatives of squalamine (also referred to herein Compound VI and ENT-06), or a pharmaceutically acceptable salt, solvate, prodrug, or derivative thereof, methods of preparing Compound VI or derivatives thereof, compositions comprising one or more of the Compound VI and derivatives thereof, and methods of using the same.

A. Overview of the Chemistry of the Disclosure

It has been known since the 1980's that 3-oxocholenic acids with specific chemical structures can be isolated from humans under specific circumstances. In particular, fluid withdrawn from a chronic subdural hematoma was shown to contain high concentrations of the bile acid Compound I, the function of which is unknown.

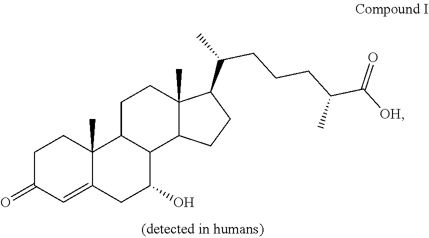 [SMILES: C[C@H](CCC[C@@H](C)[C@H]1CCC2C3C(CC[C@@]21C)[C@@]1(C)CCC(=O)C=C1C[C@H]3O)C(=O)O]

Later, this same compound was found to be present, also at high concentrations, in the cerebrospinal fluid (CSF) following subarachnoid hemorrhage, and at low concentrations in the CSF of healthy adults. Compound I, bearing a hydroxyl at the C₁₂ position, was also discovered in both the amniotic fluid and urine of newborn, healthy humans. The function of Compound I and its related metabolic variants has remained a mystery. It appears to be produced by a minor bile acid pathway that converts cholesterol initially to 27-hydroxycholesterol and then to Compound I. The role of this minor pathway remains unknown and is responsible for about 5% of bile acids produced in humans. It is believed that the products of this “acidic” pathway do not play a significant role in the emulsification of fats, the major function of bile acids in human physiology.

Although not the same, the structure of Compound I is reminiscent of the Squalus aminosterols, including those shown and discussed herein. Both steroid scaffolds are substantially flat, due to steric constraints imposed between the A and B rings. In the case of Compound I, the 4-ene double bond imparts flatness. In the shark molecules, flatness is imposed by the 5-alpha hydrogen. The carboxyl moiety of Compound I is spatially positioned similar to the sulfate on C₂₄ of the shark molecules, with both structures presenting a negative charge in the same general position in space relative to the steroid scaffold. Based on this spatial positioning, Compound VI was synthesized by coupling a protected version (Compound B) of the polyamine, spermidine with the reduced form of Compound I (Compound 4), followed by deprotection as shown below.

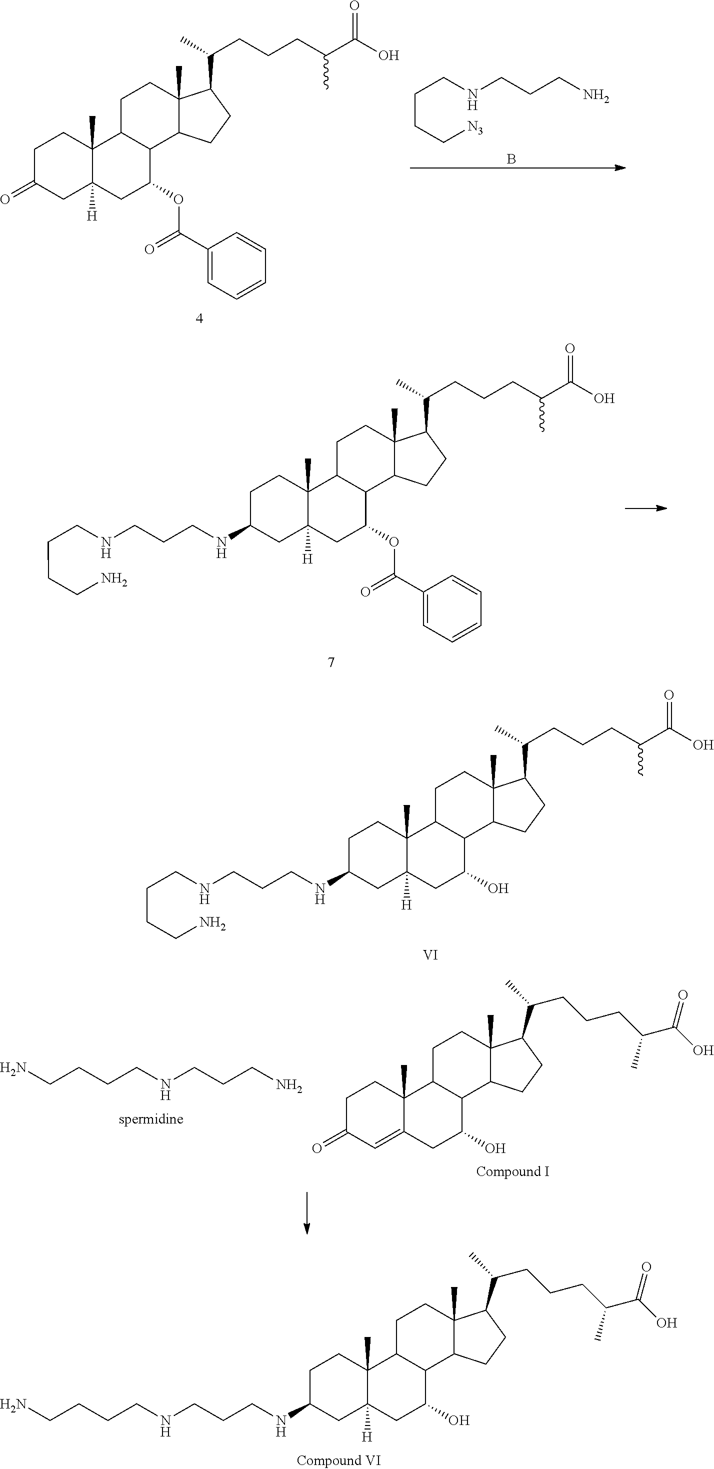

Compound VI contains a 5-α hydrogen, offering greater chemical stability versus a 4-ene such as that of Compound I. Compound VI, like the shark derived squalamine, is expected to treat abnormal alpha-synuclein pathology and/or dopaminergic dysfunction and relieve constipation in PD. Since Compound VI (ENT-06; 3-β-Spermidino-7α-hydroxy-5α-cholestanoic acid) is believed to have the same pharmacological activity known to be associated with shark derived squalamine (also referred to herein as ENT-01), the usefulness of Compound VI in other applications where squalamine, as well as other aminosterols, are known to be useful can be validated.

Compound VI contains a 5 alpha hydrogen rather than the 4-ene because of the greater chemical stability of the former versus the latter. Because of the replacement of the C24 sulfated hydroxyl on ENT-01 with the C27 carboxylic acid in ENT-06, ENT-06 will be subject to metabolism in a fashion common to all bile acids, namely via progressive cleavage of the cholesterol side chain. Differences in pharmacology are likely in part due to differences in metabolic handling of the two compounds.

B. Background Regarding Squalamine and Disease

Not to be bound by theory, it is believed that aminosterols work by targeting neurotoxic aggregates of alpha-synuclein (αS) in the gastrointestinal tract to restore function of the enteric nerve cells, thereby treating and/or preventing brain-gut disorders described herein.

Several clinical trials are in process or completed using squalamine (e.g., the shark derived compound) to treat Parkinson's Disease based upon this theory. See e.g., (1) ClinicalTrials.gov Identifier: NCT03047629 for “A Multi-Center, Single-Dose, Multiple-Dose, Double-Blind, Placebo-Controlled Study to Evaluate Safety, Tolerability, Pharmacokinetics and Pharmacodynamics of Orally Administered ENT-01 for the Treatment of Parkinson's Disease Related Constipation,” with 50 participants (study completed Jun. 14, 2018); (2) ClinicalTrials.gov Identifier: NCT03781791 for “A Multicenter, Randomized, Double-Blind, Placebo-Controlled, Multiple Dose Study to Evaluate Safety, Tolerability and Efficacy of Orally Administered ENT-01 for the Treatment of Parkinson's Disease-Related Constipation (KARMET),” with 72 participants (estimated study completion date of June 2019); and (3) ClinicalTrials.gov Identifier: NCT03938922 for “A Multicenter, Randomized, Double Blind Study to Evaluate Tolerability and Efficacy of Orally Administered ENT-01 for the Treatment of Parkinson's Disease Dementia,” with a target of 40 participants (estimated study start date of Jun. 3, 2019, and estimated study completion date of February 2020).

This effect of aminosterols is highly unexpected given that aminosterols have a very low bioavailability; e.g., squalamine appears to work locally rather than via absorption into the blood stream, thus Compound VI is believed to work locally as well. Following squalamine administration, the now-functional enteric nerve cells prevent retrograde trafficking of proteins, such as αS, to the brain. In addition to restoring gastrointestinal function, this effect is believed to slow and possibly reverse disease progression of brain-gut disorders such as Parkinson's Disease (PD), as well as other related brain-gut diseases and conditions as described herein.

A strategy that targets neurotoxic aggregates of αS in the gastrointestinal tract represents a novel approach to the treatment of brain-gut disorders such as PD and other neurodiseases and conditions described herein, and that may restore the function of enteric nerve cells and prevent retrograde trafficking to the brain. Such actions may potentially slow progression of the brain-gut disease in addition to restoring gastrointestinal function.

Accordingly, but not to be bound by theory, the methods described herein using the novel Compound VI or a pharmaceutically acceptable salt, solvate, prodrug, or derivative thereof, as well as compositions comprising the same, are expected to apply to the treatment and/or prevention of any of the described brain-gut diseases and symptoms described herein.

C. Alpha-Synuclein (αS) and Disease

PD correlates with the formation of toxic alpha-synuclein (αS) aggregates within the enteric nervous system (ENS) (Braak et al. 2003 (a); Braak et al. 2003 (b)). αS is a member of the synuclein family of soluble proteins (αS, β-synuclein and γ-synuclein) that are commonly present in the central nervous system (CNS) of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release chemical messengers, called neurotransmitters, from compartments known as synaptic vesicles. The release of neurotransmitters relays signals between neurons and is critical for normal brain function. αS can be seen in neuroglial cells and melanocytic cells and is highly expressed in the neuronal mitochondria of the olfactory bulb, hippocampus, striatum and thalamus.

αS aggregates to form insoluble fibrils in pathological conditions characterized by Lewy bodies, such as PD, dementia with Lewy bodies (DLB), and multiple system atrophy (MSA). These disorders are known as synucleinopathies. αS pathology is also found in both sporadic and familial cases with AD. Thus, one indicator of αS pathology is the formation of αS aggregates.

At the molecular level, protein misfolding, accumulation, aggregation and subsequently the formation of amyloid deposits are common features in many neurological disorders including Alzheimer's disease (AD) and PD. Thus, neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies (Brundin et al., 2017). The α-synuclein abnormalities typically found in PD are believed to be responsible for apparent catecholamine-deficits (dopamine is a catecholamine sharing metabolic pathways with other catecholamines) (Frisina et al., 2009).

Examples of conditions associated with abnormal αS pathology, and/or dopaminergic dysfunction, also referred to as “brain-gut” disorders, include but are not limited to, synucleinopathies, neurodiseases, psychological and/or behavior disorders, cerebral and general ischemic disorders, and/or disorders or conditions. Examples of synucleinopathies, neurodegenerative disease and/or neurological diseases include, for example, AD, PD, Lewy body dementia (LBD) or dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy (SMA), progressive nuclear palsy, supranuclear palsy, frontotemporal dementia (FTD), progressive supranuclear palsy, Guadeloupian Parkinsonism, parkinsonism, spinocerebellar ataxia, stroke, traumatic brain injury, degenerative processes associated with aging, and dementia of aging. Examples of psychological or behavior disorders include for example depression, autism, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, and sleep disorders such as REM sleep behavior disorder (RBD), sleep fragmentation, REM behavior disorder, circadian rhythm dysfunction, sleep apnea, and cognitive impairment. Examples of general ischemic or cerebral ischemic disorders include for example microangiopathy, intrapartum, cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure and pulmonary edema.

Constipation serves as an early indicator of many neurodiseases such as PD to the extent that it is suspected to correlate with the formation of toxic αS aggregates within the enteric nervous system (ENS) (Braak et al. 2003 (b)). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may therefore reduce the continuing neuro disease process in both the ENS and CNS (Phillips et al. 2008). This relationship between the ENS and CNS is sometimes described herein as “brain-gut” in relation to a class of disorders or the axis of aminosterol activity.

Not to be bound by theory, it is believed that aminosterols improve bowel function by acting locally on the gastrointestinal tract (as supported by the low oral bioavailability, e.g., less than about 0.3%). An orally administered aminosterol such as squalamine stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

It is theorized that nerve impulses initiated from the ENS following administration of an aminosterol augments afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of aminosterol administration, the neurons of the CNS gradually re-accumulate an αS burden either locally or via trafficking from αS re-aggregation within the gut.

Disturbance of the circadian rhythm has been described in neurodiseases such as PD both clinically and in animal models and might play a role in the abnormal sleep architecture, dementia, mood and autonomic dysfunction associated with neurodiseases such as PD (Breen et al. 2014; Videnovic et al. 2017; Antonio-Rubio et al. 2015; Madrid-Navarro et al. 2018). Circadian cycles of wrist skin temperature have been shown to correlate with sleep wake cycles, reflecting the impact of nocturnal heat dissipation from the skin on the decrease in core temperature and the onset of sleep (Sarabia et al. 2008; Ortiz-Tudela et al. 2014).

It is believed that administration of Compound VI or a salt or derivative thereof will have a significant positive impact on the circadian rhythm of patients. Not to be bound by theory, it is believed that Compound VI can affect neuronal circuits involving the master clock (the suprachiasmatic nucleus) and its autonomic projections and opens the possibility of therapeutic correction of circadian dysfunction. As described in greater detail herein, Compound VI dosing can range from about 0.01 to about 500 mg/day, with dosage determination described in more detail below.

D. Mucosal Tissue Rejuvenation

Example 1 shows that squalamine is effective at rejuvenating mucosal tissue and increasing the transcriptome in old mice. As discussed in section I.A, Compound VI and the shark-derived aminosterols such as squalamine share similar structures and spatial positioning of functional groups. Thus, the activity of squalamine is believed to extend to Compound VI and derivatives thereof.

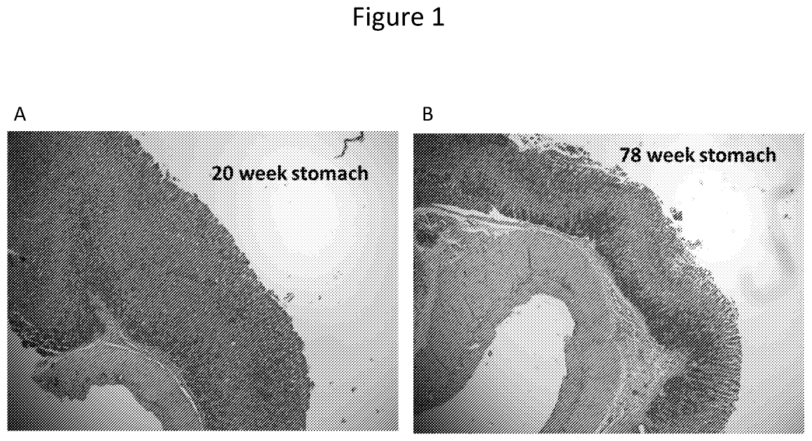

Aging involves a depletion of gene expression in the gut. Comparison of the images showing mucosal tissue in the stomach of a young mouse (20 week, FIG. 1A) versus an old mouse (78 week, FIG. 1B) shows a reduced thickness of the mucosal layer in the older specimen. This reduction in mucosa is associated with a reduced RNA transcriptome in the stomach in aged mice (e.g., 78 weeks) vs. young mice (e.g., 20 weeks), see Table 2. Example 1 evaluated the impact of oral dosing of squalamine (ENT-01) on old mice. After 2 weeks of dosing, the animals were euthanized, and the GI tracts sectioned into stomach, duodenum, jejunum, ileum, caecum, colon, and rectum. The stomach tissues were then sent for histology, and the transcriptomes analyzed by RNAseq.

mRNA levels for all of the genes of Table 3 in Example 1 showed a significant increase after treatment with squalamine. This suggests that squalamine has a rejuvenating effect in the aged gut and this activity would be believed to extend to Compound VI and salts, solvates, derivatives, and prodrugs thereof.

### II. Compounds

In one aspect an aminosterol is provided having the formula:

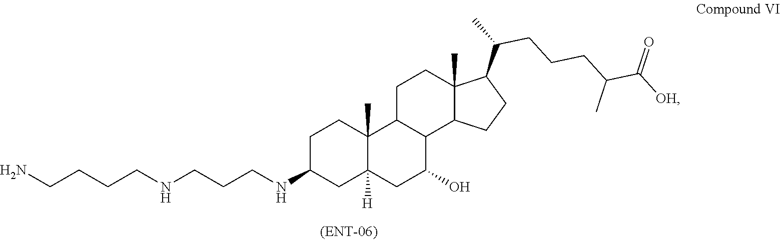 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]

or a pharmaceutically acceptable salt, solvate, prodrug, or derivative thereof.

In one embodiment the aminosterol has the formula:

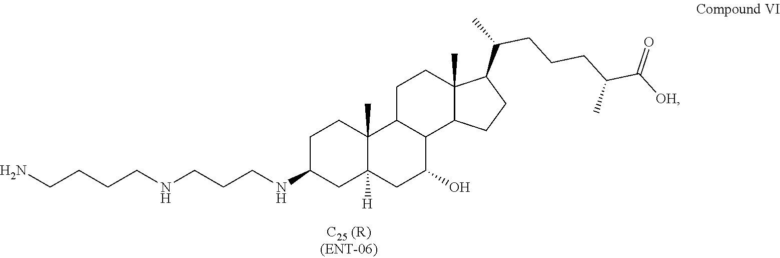 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

or a pharmaceutically acceptable salt, solvate, prodrug, or derivative thereof.

In one aspect an aminosterol is provided having the formula:

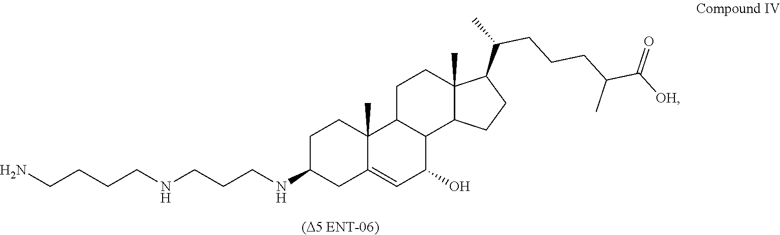 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

or a pharmaceutically acceptable salt, solvate, prodrug, or derivative thereof.

In one embodiment, the aminosterol has the formula:

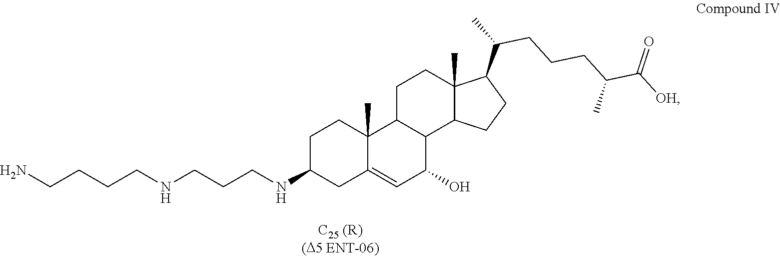 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]

or a pharmaceutically acceptable salt, solvate, prodrug, or derivative thereof.

In another aspect, an aminosterol compound having the formula:

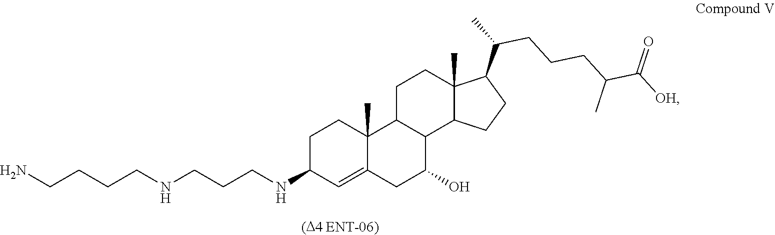 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, or a pharmaceutically acceptable salt, solvate, prodrug, or derivative thereof.

In some embodiments, the aminosterol compound has the formula:

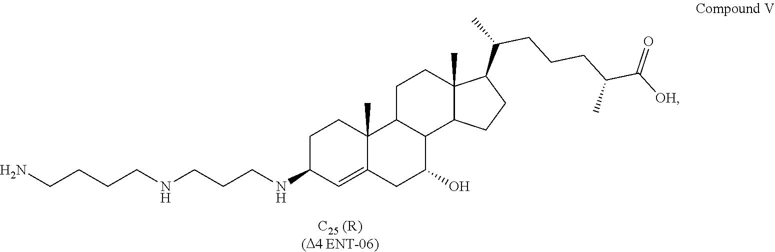 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]

or a pharmaceutically acceptable salt, solvate, prodrug, or derivative thereof.

In one embodiment, the prodrug comprises a compound of formula:

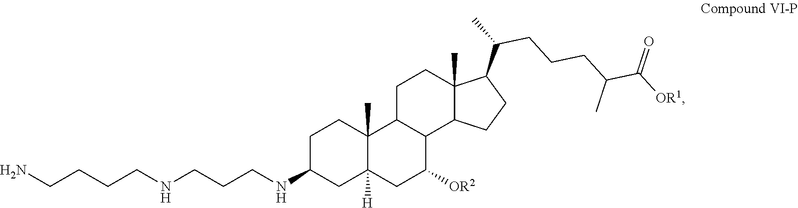

wherein:

R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and

R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl;

provided that at least one of R¹ and R² is not H,

or a pharmaceutically acceptable salt, solvate, prodrug, or derivative thereof.

In one embodiment, the prodrug comprises a compound of formula:

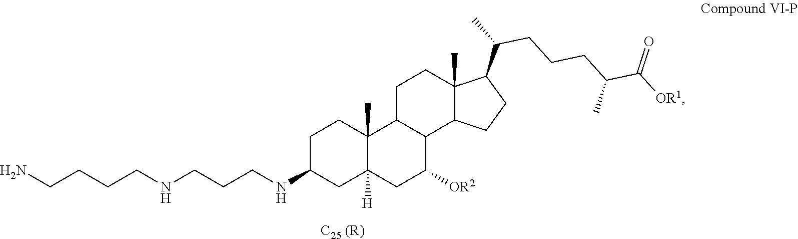

or a pharmaceutically acceptable salt, solvate, prodrug, or derivative thereof.

In one aspect, the prodrug comprises a compound having the formula:

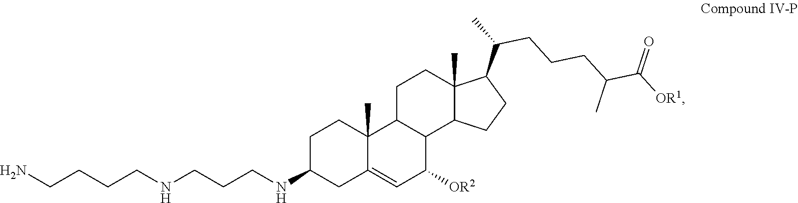

wherein:

R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and

R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl;

provided that at least one of R¹ and R² is not H,

or a pharmaceutically acceptable salt, solvate, prodrug, or derivative thereof.

In one embodiment, the prodrug comprises a compound of formula:

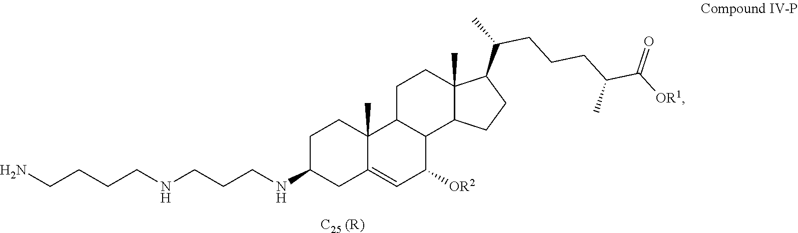

or a pharmaceutically acceptable salt, solvate, prodrug, or derivative thereof.

In another aspect, an aminosterol compound having the formula:

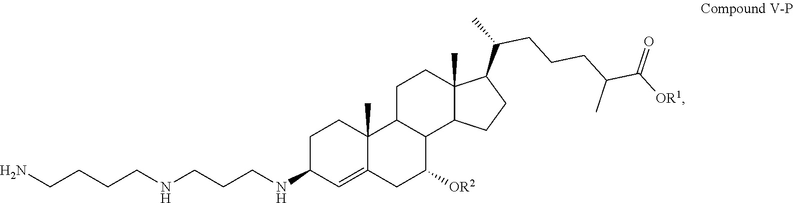

is provided wherein: R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; provided that at least one of R¹ and R² is not H, or a pharmaceutically acceptable salt, solvate, prodrug, or derivative thereof.

In one embodiment, the aminosterol compound has the formula:

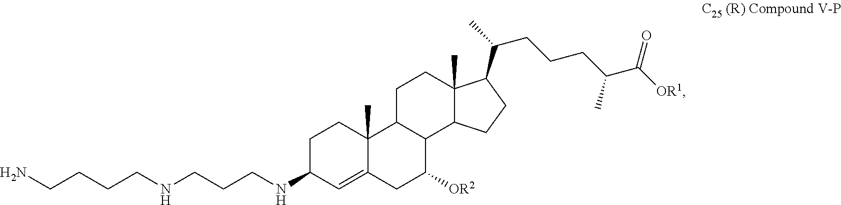

or a pharmaceutically acceptable salt, solvate, prodrug, or derivative thereof.

The aminosterols of the present disclosure may comprise an asymmetric carbon atom. As such, aminosterols of this disclosure can exist as either individual enantiomers, or mixtures of the two enantiomers. Accordingly, an aminosterol of the present disclosure can include both racemic mixtures, and also individual respective stereoisomers or diastereoisomers that are substantially free from another possible stereoisomer. The term “substantially free of other stereoisomers” as used herein means less than about 25%, less than about 20%, less than about 15%, less than about 10%, less than about 5%, less than about 4%, less than about 3%, less than about 2%, or less than about 1% of other stereoisomers, or less than “about X”% of other stereoisomers (wherein X is a number between 0 and 100, inclusive) are present.

In some embodiments, the aminosterol is a derivative of any of the aminosterols disclosed herein, or a derivative of Compound VI (ENT-06), modified through medical chemistry to improve biodistribution, ease of administration, metabolic stability, or any combination thereof. In some embodiments, Compound VI or the derivative aminosterol is modified to include one or more of the following: (1) substitutions of the carboxylate by a sulfonate, phosphate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and/or (3) substitution of various ring hydrogen atoms to prevent oxidative or reductive metabolism of the steroid ring system.

The present technology also provides salts, solvates and hydrates of the aminosterols disclosed herein. A salt of an aminosterol of this technology is formed between an acid and a basic group of the aminosterol, such as an amino functional group, or a base and an acidic group of the aminosterol, such as a carboxyl functional group. According to another embodiment, the aminosterol is a pharmaceutically acceptable acid addition salt. Examples of pharmaceutically acceptable salts include, but are not limited to, hydrochloride, sodium, sulfate, acetate, phosphate or diphosphate, chloride, potassium, maleate, calcium, citrate, mesylate, nitrate, tartrate, aluminum, and gluconate.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosutfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-I,6-dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid, hydrobromic acid, and phosphoric acid.

### III. Methods of Making the Claimed Aminosterol Compounds

In one aspect, a method of producing an aminosterol of formula:

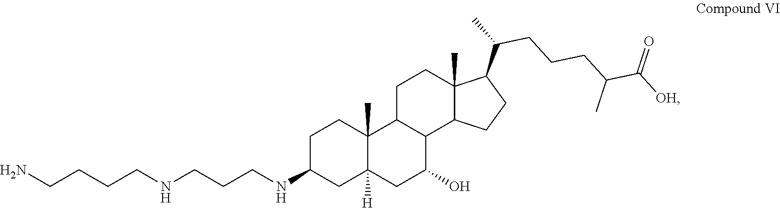 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]

is provided, the method comprising stimulating the addition of spermidine to Compound Ia:

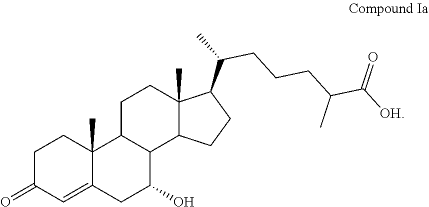 [SMILES: CC(CCC[C@@H](C)[C@H]1CCC2C3C(CC[C@@]21C)[C@@]1(C)CCC(=O)C=C1C[C@H]3O)C(=O)O]

In another aspect, a method of suppressing the formation of an aminosterol of formula:

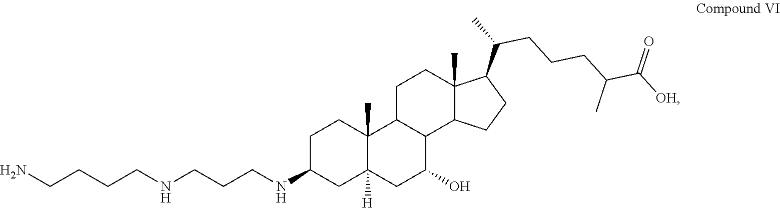 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]

is provided, the method comprising suppressing the addition of spermidine to Compound Ia:

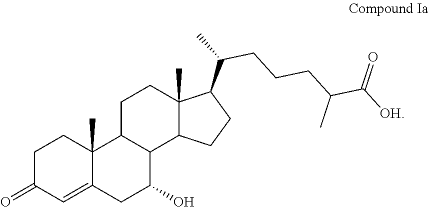 [SMILES: CC(CCC[C@@H](C)[C@H]1CCC2C3C(CC[C@@]21C)[C@@]1(C)CCC(=O)C=C1C[C@H]3O)C(=O)O]

In some embodiments, Compound Ia has the formula:

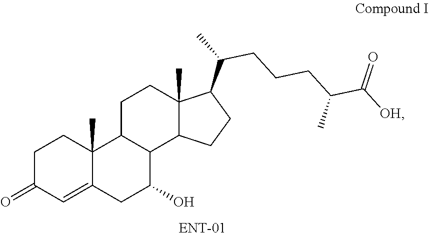 [SMILES: C[C@H](CCC[C@@H](C)[C@H]1CCC2C3C(CC[C@@]21C)[C@@]1(C)CCC(=O)C=C1C[C@H]3O)C(=O)O]

and Compound VI has the formula:

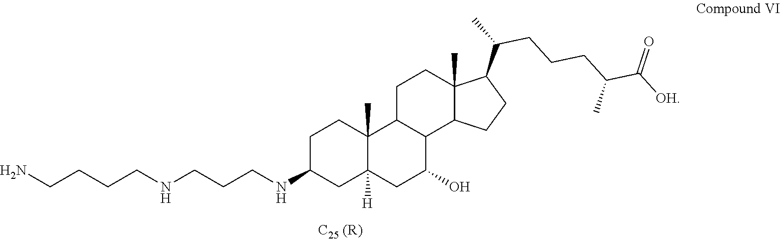 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

In another aspect, a method of producing an aminosterol of formula:

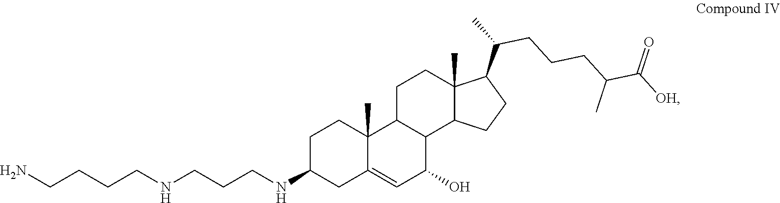 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

is provided, the method comprising stimulating the addition of spermidine to Compound Ia:

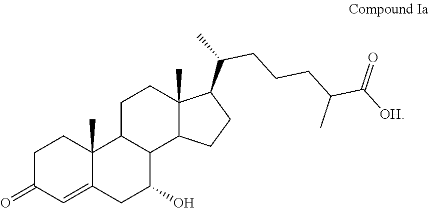 [SMILES: CC(CCC[C@@H](C)[C@H]1CCC2C3C(CC[C@@]21C)[C@@]1(C)CCC(=O)C=C1C[C@H]3O)C(=O)O]

In another aspect, a method of suppressing the formation of an aminosterol of formula:

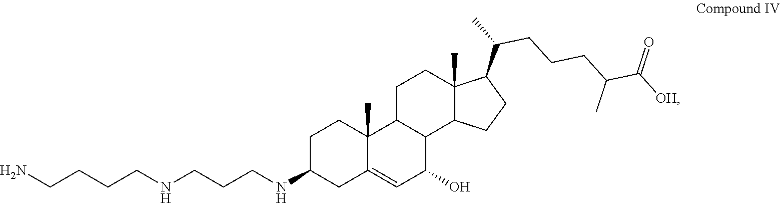 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]

is provided, the method comprising suppressing the addition of spermidine to Compound Ia:

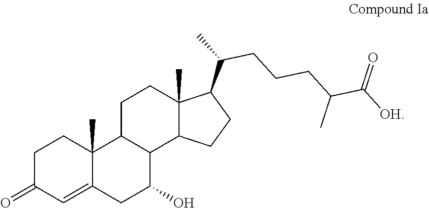 [SMILES: CC(CCC[C@@H](C)[C@H]1CCC2C3C(CC[C@@]21C)[C@@]1(C)CCC(=O)C=C1C[C@H]3O)C(=O)O]

In some embodiments, Compound Ia has the formula:

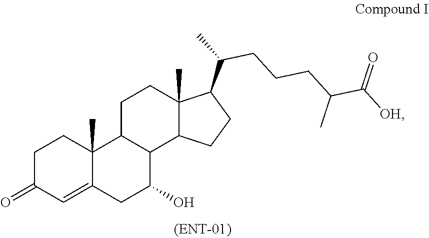 [SMILES: CC(CCC[C@@H](C)[C@H]1CCC2C3C(CC[C@@]21C)[C@@]1(C)CCC(=O)C=C1C[C@H]3O)C(=O)O]

and Compound IV has the formula:

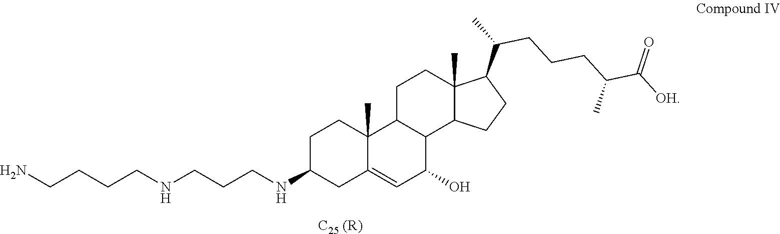 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]

In another aspect, a method of producing an aminosterol of formula:

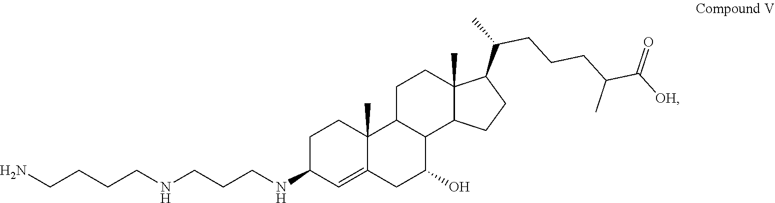 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, the method comprising stimulating the addition of spermidine to Compound Ia:

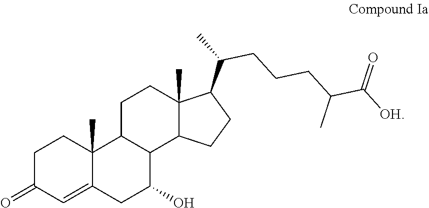 [SMILES: CC(CCC[C@@H](C)[C@H]1CCC2C3C(CC[C@@]21C)[C@@]1(C)CCC(=O)C=C1C[C@H]3O)C(=O)O]

In another aspect, a method of suppressing the formation of an aminosterol of formula:

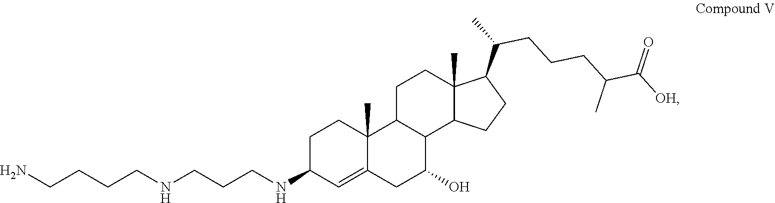 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]

is provided, the method comprising suppressing the addition of spermidine to Compound Ia:

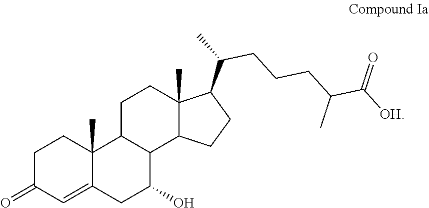 [SMILES: CC(CCC[C@@H](C)[C@H]1CCC2C3C(CC[C@@]21C)[C@@]1(C)CCC(=O)C=C1C[C@H]3O)C(=O)O]

In some embodiments, Compound Ia has the formula:

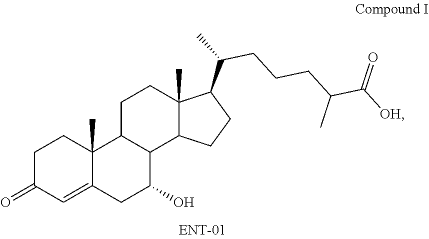 [SMILES: CC(CCC[C@@H](C)[C@H]1CCC2C3C(CC[C@@]21C)[C@@]1(C)CCC(=O)C=C1C[C@H]3O)C(=O)O]

and Compound V has the formula:

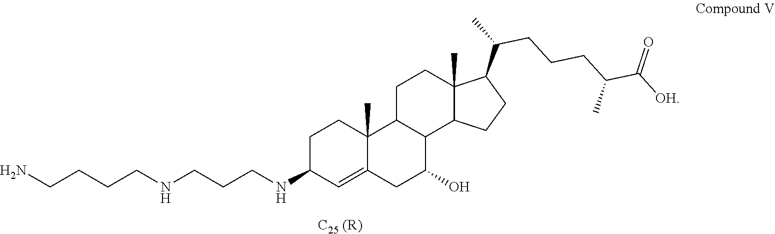 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]

In some embodiments, the aminosterol is produced in vivo in a subject. In some embodiments, the aminosterol is produced in vitro.

In some embodiments, the addition of spermidine to Compound I is suppressed in vivo in a subject. In some embodiments, the addition of spermidine to Compound I is suppressed in vitro. In some embodiments, the addition of spermidine to Compound Ia is suppressed in vivo in a subject. In some embodiments, the addition of spermidine to Compound Ia is suppressed in vitro.

In some embodiments, stimulating the addition of spermidine to Compound Ia comprises contacting a matrix comprising spermidine and Compound Ia with an agent that facilitates the addition of spermidine to Compound Ia. In some embodiments, stimulating the addition of spermidine to Compound Ia comprises contacting a cell comprising spermidine and Compound Ia with an agent that facilitates the addition of spermidine to Compound Ia. In some embodiments, the aminosterol is produced in vivo in a subject, and stimulating the addition of spermidine to Compound Ia comprises administering to the subject an effective amount of an agent that facilitates the addition of spermidine to Compound Ia. Administration of the agent may comprise administration by any of the same routes discussed herein for administering aminosterols.

In some embodiments, an agent that facilitates the addition of spermidine to Compound Ia comprises a promoter or effector. The effector may comprise an enzyme activator, enzyme inducer, a protein, a small molecule, or a nucleic acid. In some embodiments, the agent may comprise any agent that activates or catalyzes the addition of spermidine to Compound Ia. In some embodiments, the agent comprises one or more enzymes that catalyze the addition of spermidine to Compound Ia. In some embodiments, the agent comprises one or more polynucleotides encoding enzymes that catalyze the addition of spermidine to Compound Ia, or one or more polynucleotides encoding peptide effectors. The one or more polynucleotides may comprise recombinant DNA and/or RNA. In some embodiments, the agent may comprise a vector comprising the one or more polynucleotides.

In some embodiments, the spermidine addition is an enzymatic reductive amination. In some embodiments, the spermidine addition is a synthetic reductive amination. In some embodiments, the subject is human. In some embodiments, the subject includes human and non-human animals, including mammals, as well as immature and mature animals, including human children and adults. The human subject can be an infant, toddler, school-aged child, teenager, young adult, adult, or elderly patient.

### IV. Compositions

In another aspect, provided herein are compositions comprising an aminosterol compound disclosed herein, or a pharmaceutically acceptable salt, solvate, prodrug, or derivative thereof, and one or more pharmaceutically acceptable carriers and/or excipients.

In another aspect, provided herein are compositions comprising a lactate or dilactate salt of an aminosterol compound disclosed herein, and optionally one or more pharmaceutically acceptable carriers and/or excipients. Administration of an aminosterol disclosed herein, or a pharmaceutically acceptable salt, solvate, or prodrug thereof may comprise administration of the composition.

In another aspect, deuterated forms of aminosterols compounds disclosed herein can be used in the compositions and methods of the disclosure.

A. Pharmaceutical Carriers

While it is possible for an aminosterol, or a pharmaceutically acceptable salt, solvate or prodrug thereof, to be administered alone, it is preferable to administer it as a pharmaceutical formulation, together with one or more pharmaceutically acceptable carriers. The carrier(s) must be “acceptable” in the sense of being compatible with the aminosterol, or a pharmaceutically acceptable salt, solvate or prodrug thereof, and not deleterious to the recipients thereof.

Generally, the formulations are prepared by contacting an aminosterol described herein, or a pharmaceutically acceptable salt, solvate, prodrug, or derivative thereof, uniformly and intimately with liquid carriers or finely divided solid carriers or both. Then, if necessary, the product is shaped into the desired formulation. Non-aqueous vehicles such as fixed oils and ethyl oleate are also useful herein, as well as liposomes.

The carrier suitably comprises minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as gelatin, serum albumin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

In instances where aerosol administration is appropriate, an aminosterol described herein, or a pharmaceutically acceptable salt, solvate or prodrug thereof, can be formulated as an aerosol using standard procedures. The term “aerosol” includes any gas-borne suspended phase of a compound described herein which is capable of being inhaled into the bronchioles or nasal passages, and includes dry powder and aqueous aerosol, and pulmonary and nasal aerosols. Specifically, aerosol includes a gas-born suspension of droplets of a compound described herein, as may be produced in a metered dose inhaler or nebulizer, or in a mist sprayer. Aerosol also includes a dry powder composition of a composition of the present technology suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al., Pharmacol. Toxicol. Methods, 27:143-159 (1992).

B. Dosage Forms

The aminosterol compositions may conveniently be presented in unit dosage form and may be prepared by any of the methods well known in the art of pharmacy. Exemplary aminosterol dosage forms include, but are not limited to, oral, intranasal, and injectable (IP, IV, or IM). Preferably, the aminosterol formulation is administered orally, intranasally, or a combination thereof. In yet another embodiment, administration comprises non-oral administration.

Formulations or compositions of the present technology may be packaged together with, or included in a kit with, instructions or a package insert. “Pharmaceutically acceptable carrier” refers to a non-toxic solid, semisolid or liquid filler, diluent, encapsulating material or formulation auxiliary of any type.

Pharmaceutical compositions according to the present technology may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™)

Suitable lubricants, including agents that act on the flowability of the powder to be compressed, may include colloidal silicon dioxide, such as Aerosil® 200, talc, stearic acid, magnesium stearate, calcium stearate, and silica gel.

Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Inhalation or nasal dosage forms may be administered. Inhalable, pulmonary, and/or nasal dosage forms may include aerosols, dry powders, liquids or sprays. In some embodiments, administration is with a metered dose inhaler. Inhalation refers to the delivery of the aminosterol through a respiratory passage, and/or through the subject's airways, such as the subject's nose or mouth.

A metered dose inhaler in the present context means a device capable of delivering a metered or bolus dose of drug, such as aminosterol, to the lungs. One example of the inhalation device can be a pressurized metered dose inhaler, a device which produces the aerosol clouds for inhalation from solutions, solids, and/or suspensions of drugs in chlorofluorocarbon (CFC) and/or hydrofluoroalkane (HFA) solutions.

The inhalation device can be also a dry powder inhaler. In such case, the aminosterol is inhaled in solid composition, usually in the form of a powder with particle size less than 10 micrometers in diameter or less than 5 micrometers in diameter.

The metered dose inhaler can be a soft mist inhaler (SMI), in which the aerosol cloud containing a respiratory drug can be generated by passing a solution containing the respiratory drug through a nozzle or series of nozzles. The aerosol generation can be achieved in a SMI, for example, by mechanical, electromechanical or thermomechanical process. Examples of soft mist inhalers include the Respimat® Inhaler (Boeringer Ingelheim GmbH), the AERx® Inhaler (Aradigm Corp.), the Mystic™ Inhaler (Ventaira Pharmaceuticals, Inc.) and the Aira™ Inhaler (Chrysalis Technologies Incorporated). For a review of soft mist inhaler technology, see e.g. M. Hindle, The Drug Delivery Companies Report, Autumn/Winter 2004, pp. 31-34. The aerosol for SMI can be generated from a composition comprising an aminosterol, and which optionally comprises one or more pharmaceutically acceptable excipients. The composition can be, for example, an aminosterol dispersed in any suitable media, such as for example water, ethanol or a mixture thereof. In one aspect, the diameter of the aminosterol-containing aerosol particles is less than about 10 microns, or less than about 5 microns, or less than about 4 microns.

The inhaled, pulmonary or nasal formulations can include a hydrophobic substance to reduce sensitivity to humidity. Such a hydrophobic substance is preferably leucine, which makes the particle disaggregation easier.

In case of production of a solid product in a powder form, this can occur using different techniques, well described in the pharmaceutical industry. The preparation of fine particles through spray-drying or freeze-drying represents exemplary methods according to the present disclosure.

An exemplary dose of aminosterol that can be administered in a nasal, inhalation, or pulmonary administration, and in particular for treating a microbial infection, can be for example about 50 mg or less, about 45 mg or less, about 40 mg or less, about 35 mg or less, about 30 mg or less, about 25 mg or less, about 20 mg or less, about 15 mg or less, about 14 mg or less, about 13 mg or less, about 12 mg or less, about 11 mg or less, about 10 mg or less, about 9 mg or less, about 8 mg or less, about 7 mg or less, about 6 mg or less, about 5 mg or less, about 4 mg or less, about 3 mg or less, about 2 mg or less, about 1 mg or less, and greater than about 0 mg. The dose can be delivered in a single or multiple pumps or discharges from the device.

The aminosterol dose can be administered in 20 breaths or less, or in 10 breaths or less, or than 5 breaths or less. In other aspect, the aminosterol can be administered in about 3, about 2 or about 1 breaths. The total time of a single administering event can be less than 5 minutes, or less than about 1 minute, less than about 30 seconds, or less than about 15 seconds. In addition, the aminosterol can be administered a single time per day or several times per day.

C. Dosages & Dosing Period

In other aspects of the disclosure, the dosage of an aminosterol described herein can range from about 1 to about 500 mg/day, or any amount in-between these two values. In some embodiments, a subject is administered a therapeutically effective dose of an aminosterol described herein. The therapeutically effect amount of the at least one aminosterol or a salt or derivative thereof in the methods of the disclosure can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the disclosure can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

Oral dosage of an aminosterol described herein can range from about 1 to about 500 mg/day, or any amount in-between these two values. In one embodiment, the method of administration comprises oral administration and the therapeutically effective amount of the aminosterol comprises (i) about 1 to about 300 mg/day; (ii) about 25 to about 300 mg/day; (iii) about 50 to about 300 mg/day; or (iv) about 75 to about 300 mg/day. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of the aminosterol. Examples of such intranasal aminosterol low dosages include, but are not limited to, about 0.001 to about 6 mg/day, or any amount in-between these two values. In some embodiments, the method of administration comprises nasal administration and the therapeutically effective amount of the aminosterol comprises (i) about 0.001 to about 6 mg/day; (ii) about 0.001 to about 4 mg/day; (iii) about 0.001 to about 2 mg/day. For example, the low dosage of an intranasally administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that the same dosage would not provide any pharmacological effect if administered by any other route—e.g., a “subtherapeutic” dosage, and, in addition, does not result in negative effects. For example, as described herein, Compound VI (ENT-06) has the pharmacological effects of a reduction in food intake and weight loss. Therefore, in the IN methods of the disclosure, if the aminosterol is Compound VI (ENT-06) or a salt, solvate, prodrug, or derivative thereof, then if the same IN Compound VI dosage is administered via another route, such as oral, IP, or IV, then the Compound VI dosage will not result in a noticeable reduction in food intake or noticeable weight loss. Similarly, some aminosterols are known to produce the pharmacological effects of nausea, vomiting and/or reduced blood pressure. Thus, in the IN methods of the disclosure, if the aminosterol has this effect when given IN, then if the same IN aminosterol dosage is administered via another route, such as oral, IP, or IV, then the aminosterol dosage will not result in noticeable nausea, vomiting, and/or a reduction in blood pressure. Suitable exemplary aminosterol dosages are described above. In some embodiments, intranasal administration comprises delivery of the aminosterol to the brain.

Aminosterol doses can be de-escalated (reduced) if any given aminosterol dose induces a persistent undesirable side effect, such as diarrhea, vomiting, or nausea. In another embodiment, a dose of an aminosterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment an aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

The pharmaceutical composition comprising an aminosterol or a derivative, salt, solvate, or prodrug thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g., twice daily). In an exemplary embodiment, dosing is 1×/day.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the disclosure, the aminosterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the disclosure, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

D. “Fixed Aminosterol Dose”

In one aspect, the present application relates to the discovery of a method to determine a “fixed dose” of an aminosterol described herein, that is not age, size, or weight dependent but rather is individually calibrated. The “fixed dose” obtained through this method yields highly effective results in treating the symptom(s) based on which the “fixed dose” was determined, related symptoms along the “brain-gut” axis, and the underlying disorder. Further, contemplated herein are methods of leveraging this same “fixed dose” method for methods of prevention of the underlying disorder. The present disclosure is not limited to methods whereby a fixed aminosterol dosage is determined for a specific patient.

A “fixed aminosterol dose,” also referred to herein as a “fixed escalated aminosterol dose,” which will be therapeutically effective is determined for each patient by establishing a starting dose of an aminosterol composition and a threshold for improvement of a particular symptom which is used as a tool or marker for evaluating the effectiveness of the aminosterol dosage. Following determining a starting aminosterol dosage for a particular patient, the aminosterol dose is then progressively escalated by a consistent amount over consistent time intervals until the desired improvement is achieved; this aminosterol dosage is the “fixed escalated aminosterol dosage” for that particular patient for that particular symptom. In exemplary embodiments, an orally administered aminosterol dose is escalated every about 3 to about 5 days by about 25 mg until the desired improvement is reached. Symptoms evaluated, along with tools for measuring symptom improvement, may be specifically described below, including but not limited to constipation, hallucinations, sleep disturbances (e.g. REM disturbed sleep or circadian rhythm dysfunction), cognitive impairment, depression, or alpha-synuclein aggregation.

This therapeutically effective “fixed dose” is then maintained throughout treatment and/or prevention. Thus, even if the patient goes “off drug” and ceases taking the aminosterol composition, the same “fixed dose” is taken with no ramp up period following re-initiation of aminosterol treatment.

Not to be bound by theory, it is believed that the aminosterol dose is dependent on the severity of nerve damage relating to the symptom establishing the “fixed dose” threshold—e.g. for constipation, the dose may be related to the extent of nervous system damage in the patient's gut.

Dose escalation: When determining a “fixed aminosterol dosage” for a particular patient, a patient is started at a lower dose and then the dose is escalated until a positive result is observed for the symptom being evaluated. An exemplary symptom to be evaluated can be constipation, but any symptom associated with the disease or disorder to be treated can be used as a marker for evaluating aminosterol dosage. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a patient experiencing severe constipation, defined as less than one spontaneous bowel movement (SBM) a week, the starting oral aminosterol dose can be about 150 mg/day or greater. In contrast, for a patient having moderate constipation, e.g., defined as having more than one SBM a week, the starting oral aminosterol dose can be about 75 mg/day. Thus, as an example, a patient experiencing moderate constipation can be started at an oral aminosterol dosage of about 75 mg/day, whereas a patient experiencing severe constipation can be started at an oral aminosterol dosage of about 150 mg/day.

In other embodiments, a patient experiencing moderate symptoms (for the symptom being used to calculate a fixed escalated aminosterol dose) can be started at an oral aminosterol dosage of from about 10 mg/day to about 75 mg/day, or any amount in-between these values. For example, the starting oral aminosterol dosage for a moderate symptom can be about 10, about 15, about 20, about 25, about 30, about 35, about 40, about 45, about 60, about 65, about 70, or about 75 mg/day.

In yet further embodiments, when the patient is experiencing severe symptoms (for the symptom being used to calculate the fixed escalated aminosterol dose), the patient can be started at an oral aminosterol dosage ranging from about 75 to about 175 mg/day, or any amount in-between these two values. For example, the starting oral aminosterol dosage for a severe symptom can be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day.

In some embodiments, the starting oral aminosterol dose may be about 125 mg or about 175 mg/day; again dependent on the severity of the symptom, such as constipation.

Starting IN aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In exemplary embodiments, the aminosterol dose is given periodically as needed. For example, the aminosterol dose can be given once per day. The aminosterol dose can also be given every other day, 2, 3, 4, or 5× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. For example, when the symptom being treated/measured is constipation, threshold improvement can be an increase of one SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Other symptoms that can be used as an endpoint to determine aminosterol dosage for a patient's fixed escalated aminosterol dosage are any symptom known to be associated with the disease, disorder, or condition intended to be treated. For example, neurodisease symptoms described herein and include, but are not limited to, (a) at least one non-motor aspect of experiences of daily living as defined by Part I of the Unified Parkinson's Disease Rating Scale (UPDRS), such as for example cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the UPDRS, such as for example, speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the UPDRS, such as for example, speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the UPDRS, such as for example, dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (1) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

V. Methods of Treatment and/or Prevention

Aspects of this disclosure relate to methods of treating certain symptoms and/or methods of treating and/or preventing diseases or disorders associated with one or more of these symptoms by administration of a therapeutically effective amount of an aminosterol disclosed herein (e.g., Compound VI), or a pharmaceutically acceptable salt, solvate, prodrug, or derivative thereof, optionally present in one or more pharmaceutically acceptable carriers. The therapeutically effective amount can be as described herein, which includes but is not limited to a “fixed aminosterol dosage” determined as described herein.

In one embodiment, the symptoms, diseases, and/or disorders are generally correlated with abnormal αS pathology and/or dopaminergic dysfunction, which means they are amenable to treatment with aminosterols described herein. The compositions of the present technology can be administered using any pharmaceutically acceptable method, including but not limited to oral, pulmonary, nasal, and nebularization administration. In yet another embodiment, administration comprises non-oral administration.

In some embodiments, provided herein are methods for treating a subject in need having a condition or symptom susceptible to treatment with an aminosterol, comprising administering to the subject a therapeutically effective amount of an aminosterol described herein, or a pharmaceutically acceptable salt, solvate, or prodrug thereof. In some embodiments, provided herein are methods for treating a subject in need having a condition susceptible to treatment with an aminosterol, comprising administering to the subject a therapeutically effective amount of a composition comprising or consisting essentially of an aminosterol disclosed herein, or a pharmaceutically acceptable salt, solvate, or prodrug thereof, and one or more pharmaceutically acceptable carriers and/or excipients.

Non-limiting examples of symptoms amenable to treatment with aminosterols include but are not limited to constipation, hallucinations, sleep disorders, cognitive impairment, depression, and inflammation.

Examples of diseases amenable to treatment with aminosterols are described herein and include but are not limited to those described herein, such as neurological diseases, e.g., PD, AD, MSA, schizophrenia, Huntington's disease (HD), progressive supranuclear palsy, frontotemporal dementia (FTD), vascular dementia, amyotrophic lateral sclerosis (ALS), multiple sclerosis (MS), spinal muscular atrophy (SMA), Friedreich's ataxia. In another embodiment, the aminosterols described herein and compositions comprising the same can be used in methods of treating, preventing, and/or slowing the onset or progression of psychological or behavior disorder and/or a related symptom in a subject in need is provided, In one embodiment, the psychological or behavior disorder can be, for example, depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, and cognitive impairment. In another embodiment, a method of treating, preventing, and/or slowing the onset or progression of a cerebral or general ischemic disorder and/or a related symptom in a subject in need is provided. The cerebral or general ischemic disorder can be, for example, microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, and pulmonary edema.

In one embodiment, a method of inhibiting protein tyrosine phosphatase 1B (PTP1B) is provided, comprising contacting PTP1B with at least one aminosterol disclosed herein, or pharmaceutically acceptable salt, solvate, or prodrug thereof.

Applicant has shown in Example 1, that squalamine can increase transcription in the gut of old mice, thus having a rejuvenating effect on the gut. It is believed that this activity extends to Compound VI and derivatives thereof. Thus, in another aspect, a method of increasing transcription in the gut of a subject is provided, the method comprising administering to the subject a therapeutically effective amount of an aminosterol compound of any embodiment herein, or a pharmaceutically acceptable salt, solvate, prodrug, or derivative thereof.

A. Exemplary Symptoms Correlated with Abnormal αS Pathology and/or Dopaminergic Dysfunction and Amenable to Aminosterol Treatment

(1) Constipation

In one embodiment, a method of treating, preventing, and/or slowing the onset or progression of constipation and/or a related symptom in a subject in need is provided, comprising administering to the subject a therapeutically effective amount of at least one aminosterol disclosed herein, or pharmaceutically acceptable salt, solvate, or prodrug thereof.

Constipation is a common problem worldwide, affecting 2% to 27% of the population, with most estimates varying from 12% to 20%. The prevalence of constipation increases to 30%-40% among people aged>65 years and women are disproportionately affected. In North America, 63M people meet the Rome IV criteria for constipation and in the US alone, constipation is responsible for over 2M physician visits annually. Laxatives are prescribed to 2-3M patients every year and furthermore, in most patients, the condition is chronic requiring life-long treatment.

Constipation is much more common among patients with PD than in the general population. There are 1M people suffering from PD in the US, of which roughly 60%, or 600,000 suffer from chronic constipation and in most, the condition is chronic, severe and unresponsive to standard therapy. This represents an economic burden to the individual with PD and to the healthcare system. According to the Federal Supply Schedule April 2016, available at fss.gsa.gov, the average 30-day reimbursed price for a basket of orally administered drugs for constipation is approximately $260 or $3120 per year. This represents about $1.8B of prescription laxatives just for patients with PD.

Constipation not only constitutes a major economic burden, but it also significantly affects the quality of life of the individual, contributing to social isolation and depression. Furthermore, the severity of the symptoms correlates negatively with patient reported quality of life. An effective pro-kinetic medication for individuals with constipation would be a useful addition to the currently available treatments for this condition.

Constipation is defined as a lower than normal frequency of bowel movements in a fixed duration of time (e.g. less than 3 bowel movements per week). While often dismissed as strictly a gastrointestinal symptom, constipation is believed to be an early indicator of neurodegenerative disease to the extent that ENS degeneration can be indicative of later CNS degeneration. Indeed, not to be bound by theory, but constipation is believed to be one of the earliest indicators of PD pathology. Accordingly, method embodiments disclosed herein relate to the treatment of constipation or the treatment and/or prevention of an underlying disorder associated with constipation.

Constipation is common in PD and often becomes symptomatic years before the onset of the motor dysfunction and the subsequent diagnosis of PD. There is substantial evidence that the neurodegenerative process associated with PD, namely the accumulation of toxic aggregates of alpha-synuclein, occurs within the enteric nervous system years before they appear within the brain. It is believed that the enteric nervous system (ENS), with its vast surface area, is subject to continuous insults from infectious agents and toxic substances. Although the function of alpha-synuclein is not known, inflammation within the nervous system leads to an increase in its intracellular levels. In individuals with PD the increase in alpha-synuclein leads to the formation of neurotoxic aggregates, perhaps because of a failure by the neuron (due to genetic factors) to effectively dispose of them. The aggregates of alpha-synuclein then traffic along the vagal nerve to the dorsal motor nucleus within the brainstem, and from there to more rostral structures.

The individual with PD suffers from a form of constipation that is believed to be caused principally by delayed transit through the colon. In addition, defecation is often impaired by dysfunction of the PD subject's anorectal reflex. For many individuals, bowel issues represent a significant detriment to quality of life. Failure to effectively manage this problem can also lead to bowel obstruction, especially as the terminal phase of PD approaches. A limited number of therapies have been subjected to clinical trials and they include agents that increase the fluid content of the stool, either by blocking fluid resorption or increasing the osmolar load within the intestine.

The pathophysiology of the gastrointestinal (GI) dysfunction in PD involves deposition of αS within both the ENS as well as within the brainstem. For reasons that remain unknown αS, which is a protein normally produced in neurons, forms neurotoxic intracellular aggregates in PD. Numerous studies suggest that the αS aggregate formation begins in the ENS of the PD individual many years before the onset of the motor symptoms. As a consequence of the normal retrograde neuronal trafficking that occurs within the vagus nerve, toxic αS aggregates are transported from the neurons of the ENS to the dorsal motor nucleus of the vagus, and then, gradually to sites within the brain that are involved in physical movement and balance. Because the constipation is fundamentally of an acquired neurodegenerative nature, it differs from other forms of this condition.

Examples of tools that can be used to measure and evaluate the effect of aminosterol treatment on constipation include for example: (1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives); (2) Constipation—Ease of Evacuation Scale (from 1-7, with 7=incontinent, 4=normal, and 1=manual disimpaction); (3) Bristol Stool Chart, which is a patient-friendly means of categorizing stool characteristics (assessment of stool consistency is a validated surrogate of intestinal motility) and stool diary; (4) Unified Parkinson's Disease Scale (UPSRS), section 1.11 (Constipation Problems); (5) Patient Assessment of Constipation Symptoms (PAC-SYM); and (5) Patient Assessment of Constipation Quality of Life (PAC-QOL).

Examples of characteristics of constipation that can be positively affected by aminosterol treatment include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the disclosure. Further, assessments of these characteristics are known in the art, e.g. spontaneous bowel movements (SBMs)/week, stool consistency (Bristol Stool Form Scale) (Lewis and Heaton 1997; Heaton et al. 1992), ease of passage (Ease of Evacuation Scale) (Andresen et al. 2007), rescue medication use and symptoms and quality of life related to bowel function (PAC-SYM (Frank et al. 1999) and PAC-QOL (Marquis et al. 2005)).

The methods of using a therapeutically effective dose of an aminosterol composition according to the disclosure to treat and/or prevent constipation preferably results in an increase in the number of spontaneous bowel movements per week and/or an improvement in other stool conditions. The increase can be, for example, an increase of between about 1 to about 3 spontaneous bowel movements in a week, or, optionally, full restoration of regular bowel function.

In one embodiment of the disclosure, treatment of a subject having constipation with an aminosterol in a method described herein results in an improvement of one or more characteristics of constipation. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%. Examples of constipation characteristics that can be improved by the methods of the disclosure include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Measurement of a constipation characteristic can be done using any clinically recognized scale or tool.

In one embodiment, the dose of aminosterol required to obtain a positive impact on a symptom being evaluated, referred to herein as a “fixed escalated aminosterol dose,” is patient specific. A fixed escalated aminosterol dose may not be dependent upon age, size, or weight but rather can be individually calibrated. In another embodiment, the severity of constipation correlates with a higher required “fixed escalated aminosterol dose.” It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of aminosterol required to restore normal bowel function. It is also hypothesized that gastrointestinal dysmotility in PD results from the progressive accumulation of αS in the ENS, and that aminosterol treatment can restore neuronal function by displacing αS and stimulating enteric neurons. These results demonstrate that the ENS in PD is not irreversibly damaged and can be restored to normal function.

In calibrating the fixed aminosterol dose for a specific patient, the starting dose is varied based upon the severity of the constipation. Thus, for subjects with severe constipation, e.g., subjects with 1 or less CSBM or SMB per week, oral aminosterol dosing is started at about 100 to about 150 mg/day or more (or any amount in-between these values as described herein). For subjects with less severe constipation, e.g., more than 1 CSBM or SBM per week, oral aminosterol dosing is started at about 25 to about 75/day mg (or any amount in-between these values as described herein). Dosing for both patients is then escalated by defined amounts over a defined period of time until the fixed escalated dose for the patient is identified. Aminosterol doses can also be de-escalated (reduced) if any given aminosterol dose induces a persistent undesirable side effect, such as diarrhea, vomiting, or nausea.

For example, for patients with severe constipation, a starting oral aminosterol dosage can be from 75 mg up to about 300 mg/day, or any amount in-between these two values. In other embodiments, the starting oral aminosterol dosage for severely constipated patients can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg/day. A “fixed escalated” oral aminosterol dose for a severely constipated patient is likely to range from about 75 mg up to about 500 mg/day.

For patients with less severe constipation, oral aminosterol dosing is started at about 10 to about 75 mg/day, or any amount in-between these two values as described herein. For example, starting oral aminosterol dosage for patients with moderate to mild constipation can be about 1, about 5, about 10, about 15, about 20, about 25, about 30, about 35, about 40, about 45, about 50, about 55, about 60, about 65, about 70, up to less than or equal to about 75 mg/day. A fixed escalated oral aminosterol dose for a mild or moderately constipated patient is likely to range from about 5 mg up to about 350 mg/day, or any amount in-between these two values as described herein.

(2) Hallucinations

In one embodiment, a method of treating, preventing, and/or slowing the onset or progression of hallucinations and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

A hallucination is a sensory impression or perception of an object or event, in any of the 5 senses (sight, touch, sound, smell, or taste) that has no basis in external stimulation. Hallucinations can have debilitating impact on the subject's health and life by causing harm to self or others, by making it difficult for the subject to function normally in everyday situations, and by causing sleep disruption. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination a hallucination involving the perception of a physical experience occurring within the body.

Hallucinations can be a result of psychiatric conditions or correlated with diseases, such as a neurodisease. Hallucinations, especially auditory hallucinations, are characteristic of certain psychiatric conditions such as schizophrenia, occurring in up to 70-80% of subjects. They also occur in 30-50% of individuals with borderline personality disorder. Auditory hallucinations can take control of actions or behavior and elicit violent defensive behavior or alternatively lead to self-harming behavior. They can also occur in post-partum psychosis. Auditory hallucinations can less commonly occur in severely depressed patients or even in mania. Substance abuse can also be associated with visual hallucinations. Alcohol intoxication or withdrawal, post-traumatic stress disorder (PTSD) and bereavement can also be associated with visual hallucinations.

In some cases, hallucination is the result of a psychiatric or neurological disorder. The aminosterol composition can, for example, reverse the dysfunction of the psychiatric or neurological disorder and treat the hallucination. The psychiatric disorder can be, for example, selected from the group consisting of bipolar disorder, borderline personality disorder, depression (mixed), dissociative identity disorder, generalized anxiety disorder, major depression, obsessive compulsive disorder, post-traumatic stress disorder, psychosis (NOS), schizoaffective disorder, and schizophrenia. The neurodegenerative disorder can be, for example, PD, supranuclear palsy, multi-system atrophy, Parkinsonism, Alzheimer's disease, frontotemporal dementia, amyotrophic lateral sclerosis (ALS), Huntington's disease, schizophrenia, Friedreich's ataxia, multiple sclerosis (MS), Lewy body dementia or disease, spinal muscular atrophy, frontotemporal dementia, progressive nuclear palsy, Guadeloupian Parkinsonism, spinocerebellar ataxia, or vascular dementia. In a preferred embodiment, the aminosterol compositions of the disclosure reverse the dysfunction of the neurodegenerative disorder and treat the hallucination. The neurological disorder can also be, for example, the result of (a) a brain tumor, (b) a sleep disorder such as narcolepsy, or (c) a focal brain lesion, such as occipital lobe lesions or temporal lobe lesions. In an exemplary embodiment, the temporal lobe lesion can be lesions of the uncinate gyrus, cerebral peduncles, or substantia nigra. The neurological disorder can be, for example, the result of (d) a diffuse involvement of the cerebral cortex, such as that caused by a viral infectious disease.

The diffuse involvement of the cerebral cortex can be a result of a cerebral vasculitis condition, and the viral infectious disease can be, for example, acute metabolic encephalopathies, encephalitis, or meningitis. The cerebral vasculitis condition can be caused by an autoimmune disorder, a bacterial or viral infection, or a systemic vasculitis. The autoimmune disorder can be, for example, Systemic Lupus Erythematosus (SLE).

Further still, hallucinations may be caused by a sensory loss. The sensory loss can be, for example, visual, auditory, gustatory, tactile, or olfactory. In a preferred embodiment, the aminosterol compositions of the disclosure reverse the dysfunction of the sensory loss and treat the hallucination. In another preferred embodiment, the aminosterol compositions of the disclosure reverse the dysfunction of the enteric nervous system and treats the hallucination.

The methods of using a therapeutically effective amount of an aminosterol composition according to the disclosure to treat and/or prevent hallucinations preferably result in a decrease in hallucinations. The decrease can be, for example, a reduction in occurrences of hallucinations by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The methods of the disclosure may also result in the subject being hallucination-free. The hallucination can comprise, for example, a visual, auditory, tactile, gustatory or olfactory hallucination. The improvement can be measured using any clinically recognized assessment or tool.

Examples of tools that can be used to measure and evaluate the effect of aminosterol treatment on hallucinations include for example: The University of Miami Parkinson's Disease Hallucinations Questionnaire (UM-PDHQ), Unified Parkinson's Disease Scale (UPSRS), section 1.2 (Hallucinations and Psychosis), direct questioning, Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

(3) Inflammation Related to Abnormal αS Pathology and/or Dopaminergic Dysfunction and Amenable to Aminosterol Treatment

In one embodiment, provided is a method of treating, preventing, and/or slowing the onset or progression in a subject of inflammation and/or a related symptom related to αS pathology. The method comprises administering to the subject a therapeutically effective amount of at least one aminosterol disclosed herein (e.g., Compound VI), or pharmaceutically acceptable salt, solvate, or prodrug thereof.

αS is a potent pro-inflammatory hormone. Inflammation can be blocked by either of two strategies. First, inflammation can be blocked by reducing the tissue concentration of αS by decreasing or stopping production of αS. Alternatively, inflammation can be blocked by interrupting the signaling between αS and inflammatory cells that express CD11b. The subject of the methods of the disclosure can be any mammal, including a human.

The inflammatory disease or condition caused by excessive expression of neuronal αS can be a neurodegenerative disorder (NDD), such as an alpha-synucleinopathy. Exemplary alpha-synucleinopathies include, but are not limited to, PD, Lewy body dementia, multiple system atrophy, amytrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal αS can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

In some embodiments of the disclosure, patient populations particularly susceptible to excessive production or secretion of αS can benefit from the methods of the disclosure and are targeted for therapy, including for example preventative therapy. For example, a patient population having a mutated form of αS resulting in increased amounts of αS in tissues can be treated using the methods of the disclosure. Another example of a patient population susceptible for high levels of αS are patients having chronic inflammatory conditions or diseases. A still further example is a patient population having elevated levels of αS aggregation in their enteric nerve cells, manifesting as a constipation.

The methods of the disclosure can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

In some embodiments, the decrease is measured quantitatively or qualitatively by a method selected from the group consisting of high-performance liquid chromatography, liquid chromatography mass spectrometry, enzyme linked immunosorbent assay, protein immunoprecipitation, immunoelectrophoresis, Western blot, and protein immunostaining.

In addition, it follows from the present disclosure that an individual with an inflammatory condition appropriate for treatment or prophylaxis with the methods targeting αS described herein can be identified by determination of the tissue concentrations of αS at sites of inflammation, with high levels of αS, as compared to a control or healthy subject, correlating with patients appropriate for treatment with a method of the disclosure.

It is theorized that administration of an aminosterol reduces the formation of neurotoxic αS aggregates in vivo, and stimulates gastrointestinal motility in patients with neurodiseases such as PD and constipation. It is also hypothesized that the greater the burden of αS impeding neuronal function, the higher the dose of aminosterol required to restore normal bowel function as well as address other symptoms of alpha-synuclein aggregation.

B. Exemplary Diseases or Disorders Correlated with Abnormal αS Pathology and/or Dopaminergic Dysfunction and Amenable to Aminosterol Treatment

The aminosterols described herein (e.g., Compound VI), including a pharmaceutically acceptable salt, solvate, prodrug, or derivative thereof, can be used in methods of treating and/or preventing a variety of diseases and disorders, which are generally correlated with abnormal αS pathology and/or dopaminergic dysfunction, as described herein and as described below.

In one embodiment, provided is a method of treating, preventing, and/or slowing the onset or progression in a subject of diseases or disorder correlated with abnormal αS pathology and/or dopaminergic dysfunction and/or a related symptom related to αS pathology. The method comprises administering to the subject a therapeutically effective amount of at least one aminosterol disclosed herein (e.g., Compound VI), or pharmaceutically acceptable salt, solvate, prodrug, or derivative thereof.

### (1) Neurological or Neurodegenerative Disorders or Diseases

In one embodiment, a method of treating, preventing, and/or slowing the onset or progression of a neurodegenerative disease or neurological disease and/or a related symptom in a subject in need is provided, comprising administering to the subject a therapeutically effective amount of at least one aminosterol disclosed herein, or pharmaceutically acceptable salt, solvate, prodrug, or derivative thereof.

The methods and aminosterol compositions of the disclosure can be used to treat and/or prevent neurological disorders or diseases such as those described herein, examples of which include but are not limited to AD, PD, Huntington's Disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, frontotemporal dementia, progressive nuclear palsy, Guadeloupian Parkinsonism, spinocerebellar ataxia, and autism.

A variety of neuroimaging techniques may be useful for the early diagnosis and/or measurement of progression of neurodegenerative disorders. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, “Neuroimaging in the early diagnosis of neurodegenerative disease,” Transl. Neurodegener., 1:5 (2012). Combinations of these techniques can also be used to measure disease progression.

Progression of neurodegeneration can be measured using well known techniques. For example, an electroencephalogram (EEG) can be used as a biomarker for the presence and progression of a neurodegenerative disease (Morairty, 2013). Another exemplary technique that can be used to measure progression of neurodegeneration of MRI (Rocca et al., 2017). Alternatively, neurodegeneration can be measured by measuring the levels of one or more biomarkers known in the art to indicate neurodegeneration using analytical techniques selected from the group consisting of, for example, high-performance liquid chromatography, liquid chromatography mass spectrometry, enzyme linked immunosorbent assay, protein immunoprecipitation, immunoelectrophoresis, Western blot, and protein immunostaining. Biomarkers indicating neurodegeneration are known to the skilled artisan and may include, for example, any of those in Beach et al. 2017, the entire disclosure of which is hereby incorporated by reference.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. Id.

In one embodiment of the disclosure, the progression or onset of a neurodegenerative disorder is slowed or prevented over a defined time period, following administration of a therapeutically effective amount of an aminosterol according to the disclosure to a subject in need, as measured by a medically-recognized technique. For example, the progression or onset of a neurodegenerative disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The period of time over which the progression or onset of a neurodegenerative disorder is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the disclosure, a neurodegenerative disorder may be positively impacted by administration of a therapeutically effective amount of an aminosterol according to the disclosure. A “positive impact” includes for example slowing advancement of the condition, improving one or more symptoms, etc.

(i) Parkinson's Disease

In one embodiment, a method of treating, preventing, and/or slowing the onset or progression of Parkinson's disease (PD) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

PD is a progressive neurodegenerative disorder caused by accumulation of the protein αS within the peripheral and central nervous system (CNS), including the enteric nervous system (ENS), autonomic nerves and brain (Braak et al. 2003 (a) and (b)). While motor symptoms are still required for a diagnosis of PD (Hughes et al. 1992), non-motor symptoms represent a greater therapeutic challenge (Zahodne et al. 2012). These symptoms include constipation (Ondo et al. 2012; Lin et al. 2014), disturbances in sleep architecture (Ondo et al. 2001; Gjerstad et al. 2006), cognitive dysfunction (Auyeung et al. 2012), hallucinations (Friedman et al. 2016; Diederich et al. 2009), REM behavior disorder (RBD) and depression (Aarsland et al. 2007), all of which result from impaired function of neural pathways not restored by replacement of dopamine. In fact, long-term institutionalization, caregiver burden and decrease in life expectancy correlate more significantly with the severity of these symptoms than with motor symptoms (Goetz et al. 1995). In 2003, Braak proposed that PD begins within the GI tract caused when neurotoxic aggregates of αS form within the ENS, evidenced clinically by the appearance of constipation in a majority of people with PD many years before the onset of motor symptoms (Braak et al., 2003 (a) and (b)). A recent study in rats has demonstrated movement of aggregates of αS from the ENS to the CNS via the vagus and other afferent nerves. Neurotoxic aggregates accumulated progressively within the brainstem and then dispersed rostrally to structures within the diencephalon, eventually reaching the cerebral hemispheres.

PD is the second most common age-related neurodegenerative disease after AD. PD affects over 1% of the population over the age of 60, which in the US equates to over 500,000 individuals, while in individuals over the age of 85 this prevalence reaches 5%, highlighting the impact that advancing age has on the risk of developing this condition.

PD is divided into three stages: preclinical (in which neurodegenerative process is started without evident symptoms or signs); prodromal (in which symptoms and signs are present but insufficient to define a full clinical PD diagnosis); and clinical (in which the diagnosis is achieved based on the presence of classical motor signs). The so-called gold standard for PD diagnosis entails expert diagnosis based on patient symptoms. PD and prodromal PD diagnosis is probabilistic, made on the basis of the presence of particular motor and non-motor symptoms, physiological pathologies, genetic characteristics, and environmental factors. Diagnosis may include a combination of markers (any disease indicator, whether a symptom, sign, or biomarker) ranging from mild motor symptoms [i.e., UPDRS 1987 version score≥3 excluding action tremor; or MDS-UPDRS score>6 excluding postural-action tremor; slowness, loss of muscle movements, tremor, rigidity, imbalance, abnormal posture], non-motor symptoms (i.e., REM SBD, olfactory dysfunction, constipation, excessive daytime somnolence, symptomatic hypotension, erectile/urinary dysfunction, depression, cognition), and ancillary diagnostic tests (i.e., abnormal tracer uptake of the presynaptic dopaminergic system: SPECT or positron emission tomography).

PD may also be assessed using the UPDRS, which consists of 42 items in four subscales: (1) Part I, Non-Motor Aspects of Experiences of Daily Living (nM-EDL): cognitive impairment (section 1.1), hallucinations and psychosis (section 1.2), depressed mood (section 1.3), anxious mood (section 1.4), apathy (section 1.5), features of dopamine dysregulation syndrome (section 1.6), sleep problems (section 1.7), daytime sleepiness (section 1.8), pain and other sensations (section 1.9), urinary problems (section 1.10), constipation problems (section 1.11), light headedness on standing (section 1.12), and fatigue (section 1.13); (2) Part II, Motor Aspects of Experiences of Daily Living (M-EDL): speech (section 2.1), saliva & drooling (section 2.2), chewing and swallowing (section 2.3), eating tasks (section 2.4), dressing (section 2.5), hygiene (section 2.6), handwriting (section 2.7), doing hobbies and other activities (section 2.8), turning in bed (section 2.9), tremor (section 2.10), getting out of bed, a car, or a deep chair (section 2.11), walking and balance (section 2.12), and freezing (section 2.13); Part III, Motor Examination: speech (section 3.1), facial expression (section 3.2), rigidity (section 3.3), finger tapping (section 3.4), hand movements (section 3.5), pronation-supination movements of hands (section 3.6), toe tapping (section 3.7), leg agility (section 3.8), arising from chair (section 3.9), gait (3.10), freezing of gait (section 3.11), postural stability (section 3.12), posture (section 3.13), global spontaneity of movement (body bradykinesia) (section 3.14), postural tremor of the hands (section 3.15), kinetic tremor of the hands (section 3.16), rest tremor amplitude (section 3.17), and constancy of rest tremor (section 3.18); Part IV, Motor Complications: time spent with dyskinesias (section 4.1), functional impact of dyskinesias (section 4.2), time spent in the off state (section 4.3), functional impact of fluctuations (section 4.4), complexity of motor fluctuations (section 4.5), and painful off-state dystonia (section 4.6).

Further, symptom-based endpoints can be assessed using known scales. For example, (1) depression can be assessed using the Beck Depression Inventory (BDI-II) (Steer et al. 2000), cognition can be assessed using the Mini Mental State Examination (MMSE) (Palsetia et al. 2018), sleep and REM-behavior disorder (RBD) can be assessed using a daily diary and an RBD questionnaire (RBDQ) (Stiasny-Kolster et al. 2007), and hallucinations can be assessed using the PD hallucinations questionnaire (PDHQ) (Papapetropoulos et al. 2008) and direct questioning. Circadian system status can also be assessed by continuously monitoring wrist skin temperature (Thermochron iButton DS1921H; Maxim, Dallas) following published procedures (Sarabia et al. 2008).

In another embodiment, administration of a therapeutically effective amount of an aminosterol composition described herein to a PD patient results in improvement of one or more symptoms of PD or on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

PD progression and treatment is particularly difficult in view of patients' development of resistance to dopamine and subsequent dose escalation until no response can be elicited. Not to be bound by theory, it is believed that prior or co-administration of an aminosterol composition according to the disclosure (e.g., Compound VI) may reduce the dopamine dosage required to elicit a therapeutic effect for Parkinson's symptoms and/or increase the period during which the patient is sensitive to dopamine. It is also theorized that prior or co-administration of an aminosterol composition according to the disclosure may delay the time period when a patient is advised to begin dopamine therapy. This is significant, as currently patients are encouraged to delay initiation of dopamine treatment as long as possible, as after a period of time subjects become resistant to dopamine.

(ii) Alzheimer's Disease

In one embodiment, a method of treating, preventing, and/or slowing the onset or progression of Alzheimer's disease (AD) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Alzheimer's disease (AD) is a chronic neurodegenerative disease that usually starts slowly and worsens over time. It is the cause of 60-70% of cases of dementia. As the disease advances, symptoms can include problems with language, disorientation, mood swings, loss of motivation, not managing self-care, and behavioral issues. As a person's condition declines, they often withdraw from family and society. Gradually, bodily functions are lost, ultimately leading to death. Although the speed of progression can vary, the typical life expectancy following diagnosis is 3 to 9 years. In 2015, there were approximately 29.8 million people worldwide with AD. It most often begins in people over 65 years of age, although 4% to 5% of cases are early-onset Alzheimer's. It affects about 6% of people 65 years and older. In 2015, dementia resulted in about 1.9 million deaths.

The symptoms of Alzheimer's disease are primarily marked by cognitive deficits including memory impairment, language dysfunction, and visuospatial skills; functional impairment that may span occupational and social issues (e.g., activities of daily living); and behavioral symptoms including depression, anxiety, aggression and psychosis may also appear as the disease progresses in severity.

At this time, unambiguous diagnosis of AD requires clinical findings of cognitive deficits consistent with AD and post-mortem identification of brain pathologies consistent with AD. The term AD dementia is used to describe dementia that is due to the pathophysiologies of AD. The term “probable Alzheimer's disease” is used in life when a subject demonstrates clinical characteristics of AD and when other possible biological causes of dementia (e.g. PD or stroke) are excluded. There are currently a variety of art-accepted methods for diagnosing probable AD. Typically, these methods are used in combination and include determining an individual's ability to carry out daily activities and identifying changes in behavior and personality. Dementia of the AD type is also typically characterized by an amnestic presentation (memory deficit) or language, visuospatial or executive function deficits. Cognitive ability/impairment may be determined by art-accepted methods, including, but not limited to, validated instruments that assess global cognition (e.g., the Modified Mini Mental State Examination (3MS-E)), and specific domains such as visual and verbal memory (e.g., the Brief Visuospatial Memory Test (Revised) (BVMT-R) and the Hopkins Verbal Learning Test (Revised) (HVLT-R), respectively), language (e.g., the Generative Verbal Fluency Test (GVFT)) and executive function and attention (e.g., the Digit Span Test (DST)). Dementia due to AD is also defined by insidious onset and a history of worsening cognitive performance.

The criteria for ‘probable AD’ are described a National Institute of Aging-Alzheimer's Association workgroup (McKhann et al. 2011). According to this workgroup, for people who first exhibit the core clinical characteristics of AD dementia, evidence of biomarkers associated with the disease may enhance the certainty of the diagnosis.

In another embodiment, administration of a therapeutically effective amount of an aminosterol composition to an AD patient results in improvement of one or more symptoms of AD or on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

(iii) Multiple System Atrophy

In one embodiment, a method of treating, preventing, and/or slowing the onset or progression of multiple system atrophy (MSA) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Multiple system atrophy (MSA) is a progressive neurodegenerative disorder characterized by a combination of symptoms that affect both the autonomic nervous system (the part of the nervous system that controls involuntary action such as blood pressure or digestion) and movement. MSA, also known as Shy-Drager syndrome, is a neurodegenerative disorder characterized by tremors, slow movement, muscle rigidity, and postural instability (collectively known as parkinsonism) due to dysfunction of the autonomic nervous system, and ataxia. This is caused by progressive degeneration of neurons in several parts of the brain including the substantia nigra, striatum, inferior olivary nucleus, and cerebellum. There is no known cure for MSA and management is primarily supportive.

Progression of neurodegeneration can be measured using well known techniques. For example, an electroencephalogram (EEG) can be used as a biomarker for the presence and progression of a neurodegenerative disease (Morairty, 2013). Another exemplary technique that can be used to measure progression of neurodegeneration of MRI. Rocca et al. 2017.

A variety of neuroimaging techniques may be useful for the early diagnosis and/or measurement of progression of MSA. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for MSA progression), multimodal imaging, and biomarker analysis (Stoessl, 2012). Combinations of these techniques can also be used to measure disease progression.

For example, structural MRI can be used to measure atrophy of the hippocampus and entorhinal cortex in MSA, as well as involvement of the lateral parietal, posterior superior temporal and medial posterior cingulate cortices. In frontotemporal dementias (FTD), structural MRI can show atrophy in frontal or temporal poles.

In another embodiment, administration of a therapeutically effective amount of an aminosterol composition to an MSA patient results in improvement of one or more symptoms of MSA, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool or assessment.

(iv) Schizophrenia

In one embodiment, a method of treating, preventing, and/or slowing the onset or progression of schizophrenia (SZ) and/or a related symptom in a subject in need is provided, comprising administering to the subject a therapeutically effective amount of at least one aminosterol disclosed herein (e.g., Compound VI) or pharmaceutically acceptable salt, solvate, prodrug, or derivative thereof.

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease.

The mainstay of treatment is antipsychotic medication, along with counselling, job training and social rehabilitation. However, the 2013 study found that in general, the higher the anti-psychotic medication doses, the greater the loss of brain tissue.

About 0.3-0.7% of people are affected by schizophrenia during their lifetimes. In 2013 there were an estimated 23.6 million cases globally. Males are more often affected, and on average experience more severe symptoms. About 20% of people do well and a few recover completely. About 50% have lifelong impairment. Social problems, such as long-term unemployment, poverty and homelessness are common. The average life expectancy of people with the disorder is ten to twenty-five years less than for the general population. This is the result of increased physical health problems and a higher suicide rate (about 5%). In 2015 an estimated 17,000 people worldwide died from behavior related to, or caused by, schizophrenia.

While not wished to be bound by theory, it is theorized that administration of a therapeutically effective amount of an aminosterol composition to a schizophrenia patient may treat and/or prevent schizophrenia or any one or more symptoms thereof. In some embodiments, the administration may be oral, resulting in absorption in the ENS. In some embodiments, the administration may be intranasal, resulting in stimulation of neurogenesis, which has a positive impact on the loss of brain tissue characteristic of schizophrenia subjects.

In one embodiment of the disclosure, administration of a therapeutically effective amount of an aminosterol composition to a schizophrenia patient results in improvement of one or more symptoms as determined by a clinically recognized psychiatric symptom rating scale. Examples of such rating scales include for example, the Positive and Negative Syndrome Scale (PANSS), the Psychotic Symptom Rating Scales (PSYRATS), the Quality of Life Scale (QLS), the Schizophrenia Cognition Rating Scale (SCoRS), the Drug Attitude Inventory (DAI), and the Abnormal Involuntary Movement Scale (AIMS).

In another embodiment, administration of a therapeutically effective amount of an aminosterol composition to a schizophrenia patient results in improvement of one or more symptoms as determined by a clinically recognized psychiatric symptom rating scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool, scale, or assessment.

(v) Other Neurodiseases

The methods and compositions of the disclosure may also be useful in treating and/or preventing a variety of other neurodiseases. In one embodiment, a method of treating, preventing, and/or slowing the onset or progression of a neurodisease described herein, and/or a related symptom, in a subject in need is provided, comprising administering to the subject a therapeutically effective amount of at least one aminosterol disclosed herein (e.g., Compound VI), or pharmaceutically acceptable salt, solvate, prodrug, or derivative thereof. Examples of exemplary neurodiseases are described below and herein.

Huntington's disease (HD) is a fatal genetic disorder that causes the progressive breakdown of nerve cells in the brain. It deteriorates a person's physical and mental abilities during their prime working years and has no cure. Full-time care is required in the later stages of the disease. Symptoms of Huntington's disease most commonly become noticeable between the ages of 35 and 44 years, but they can begin at any age from infancy to old age. The most characteristic initial physical symptoms are jerky, random, and uncontrollable movements called chorea. Suicide is the cause of death in about 9% of cases. Death typically occurs 15 to 20 years from when the disease was first detected.

Progressive supranuclear palsy, also called Steele-Richardson-Olszewski syndrome, is a brain disorder that causes serious problems with walking, balance and eye movements. The disorder results from deterioration of cells in areas of the brain that control body movement and thinking. There is no known cure for PSP and management is primarily supportive.

Frontotemporal dementia (FTD) is a group of related conditions resulting from the progressive degeneration of the temporal and frontal lobes of the brain. These areas of the brain play a significant role in decision-making, behavioral control, emotion and language. The frontotemporal dementias (FTD) encompass six types of dementia involving the frontal or temporal lobes. They are: behavioral variant of FTD, semantic variant primary progressive aphasia, nonfluent agrammatic variant primary progressive aphasia, corticobasal syndrome, progressive supranuclear palsy, and FTD associated with motor neuron disease. Currently, there is no cure for FTD.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes.

Amyotrophic lateral sclerosis (ALS), also known as motor neuron disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. While the cause is not clear, the underlying mechanism is thought to be either destruction by the immune system or failure of the myelin-producing cells. Proposed causes for this include genetics and environmental factors such as being triggered by a viral infection. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. SMA is the most common genetic cause of infant death. In December 2016, nusinersen became the first approved drug to treat SMA while several other compounds remain in clinical trials.

Friedreich's ataxia is an autosomal recessive inherited disease that causes progressive damage to the nervous system. It manifests in initial symptoms of poor coordination such as gait disturbance; it can also lead to scoliosis, heart disease and diabetes, but does not affect cognitive function. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses).

### (2) Psychological or Behavior Disorders and/or a Related Symptom

In one embodiment, a method of treating, preventing, and/or slowing the onset or progression of psychological or behavior disorder and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof. In one embodiment, the psychological or behavior disorder is depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, or cognitive impairment.

(i) Depression

In one embodiment, a method of treating, preventing, and/or slowing the onset or progression of depression and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Clinical depression is characterized by a sad, blue mood that goes above and beyond normal sadness or grief. Major depression is an episode of sadness or apathy along with other symptoms that lasts at least two consecutive weeks and is severe enough to interrupt daily activities. Depressive events feature not only negative thoughts, moods, and behaviors but also specific changes in bodily functions (like, eating, sleeping, energy and sexual activity, as well as potentially developing aches or pains). One in 10 people will have a depression in their lifetime. Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the worki6yng brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the disclosure, encompassed are methods of treating and/or preventing depression comprising administering therapeutically effective amount of an aminosterol composition according to the disclosure. While not wishing to be bound by theory, it is theorized that the aminosterol compositions of the disclosure trigger neurogenesis, which functions to combat depression.

In some embodiments, the methods of the disclosure produce an improvement in a subject's clinical depression. An improvement in a subject's depression can be measured using any clinically-recognized measurement. For example, improvement can be measured using a depression rating scale. In one embodiment of the disclosure, following treatment a subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement. The improvement can be measured using any clinically recognized tool or assessment.

Examples of tools that can be used to evaluate depression and/or mood and the improvement following aminosterol treatment include for example: (1) Beck Depression Inventory (BDI-II); (2) UPDRS, sections 1.3 (depressed mood), 1.4 (anxious mood), 1.5 (apathy), and 1.13 (fatigue); and (3) Parkinson's Disease Fatigue Scale (PFS-16). In some embodiments, the improvement can be measured from one or more medically-recognized techniques selected from the group consisting of the Patient Health Questionnaire-9 (PHQ-9); Zung Self-Rating Depression Scale; Center for Epidemiologic Studies-Depression Scale (CES-D); and the Hamilton Rating Scale for Depression (HRSD).

(ii) Cognitive Impairment

In one embodiment, a method of treating, preventing, and/or slowing the onset or progression of cognitive impairment and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Cognitive impairment, including mild cognitive impairment (MCI), is characterized by increased memory or thinking problems exhibited by a subject as compared to a normal subject of the same age. Approximately 15 to 20% of people age 65 or older have MCI, and MCI is especially linked to neurodegenerative conditions such as AD and synucleinopathies like PD. In 2002, an estimated 5.4 million people (22%) in the United States over age 70 had cognitive impairment without dementia. Plassman et al. 2009.

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Related disorders and conditions include, but are not limited to, dementia, Alzheimer's, delirium, PD, diabetes, high blood pressure, high cholesterol, depression, psychological and behavioral conditions, amnesia, Lewy body diseases, and Huntington's disease, among others.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al. 2011; McKhann et al. 2011; Albert et al. 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used. Palsetia et al. (2018); Kirkevold, O. & Selbaek, G. (2015). With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F. 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective amount of an aminosterol composition to a patient in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Other examples of tools that can be used to evaluate cognitive impairment and the improvement following aminosterol treatment include for example: (1) Mini Mental State Examination (MMSE); (2) Trail Making Test (TMT) Parts A and B; and (3) UPDRS, sections 1.1 (cognitive impairment).

### (iii) Sleep Disturbance/Sleep Problems (e.g., REM Disturbed Sleep or Circadian Rhythm Dysfunction)

In one embodiment, a method of treating, preventing, and/or slowing the onset or progression of a sleep disturbance, sleep problem, sleep disorder, circadian rhythm dysfunction, and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Normal sleep is critically important for the proper functioning of many organ systems, the most important of which is the brain. Disturbances in normal sleep patterns are closely associated with the normal aging process, with the development of cognitive impairment, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “zeitgeber” or circadian clock. These conditions may be reversible, such as desynchronization resulting from jet-lag, night-shift work or hunger, conditions easily remedied by adaptation or food intake. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to REM-behavior disorders, disturbances in the Circadian rhythm (“circadian rhythm dysfunction”), delayed sleep onset, sleep fragmentation, REM-behavior disorder” (RBD), and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” (RBD) or “REM-disturbed sleep”, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

A “normal” or “restful” sleep period is defined as a sleep period uninterrupted by wakefulness. Alternatively, a said period can be defined by the recommended or appropriate amount of sleep for the subject's age category, e.g., (i) infants β-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults≥65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. Similar recommendations are provided by the National Sleep Foundation (https://sleepfoundation.org/press-release/national-sleep-foundation-recommends-new-sleep-times/page/0/1):

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008. Similarly, symptoms of RBD can be monitored using a daily diary and RBD questionnaire (Stiasny-Kolster et al. 2007).

In some embodiments, administration of a therapeutically effective amount of an aminosterol composition to a patient with disturbed results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Examples of tools that can be used to measure and evaluate the effect of aminosterol treatment on sleep include for example: (1) Sleep Diary (participants completed a sleep diary on a daily basis throughout the study. The diaries included time into bed and estimated time to sleep as well as wake time and duration during the night); (2) I-Button Temperature Assessment; (3) UPDRS, sections 1.7 (sleep problems), 1.8 (daytime sleepiness) and 1.13 (fatigue); (4) Parkinson's Disease Fatigue Scale (PFS-16); (5) REM Sleep Behavior Disorder Screening Questionnaire; and (6) Parkinson's Disease Sleep Scale.

(iv) Autism

In one embodiment, a method of treating, preventing, and/or slowing the onset or progression of autism spectrum disorder (ASD) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Autism, or autism spectrum disorder, refers to a range of conditions characterized by challenges with social skills, repetitive behaviors, speech and nonverbal communication, as well as by unique strengths and differences. There are many types of autism, caused by different combinations of genetic and environmental influences. Autism's most-obvious signs tend to appear between 2 and 3 years of age. In some cases, it can be diagnosed as early as 18 months. Some developmental delays associated with autism can be identified and addressed even earlier.

Experts are still uncertain regarding the causes of autism. In all likelihood, there are multiple causes. It appears that a number of different circumstances, including environmental, biologic, and genetic factors, set the stage for autism and make a child more likely to have the disorder. It is likely that genetics play a large factor in the development of autism. Identical twins are more likely to both be affected than twins who are fraternal (not genetically identical). In a family with one autistic child, the chance of having another child with autism is about 5%, which is much higher than in the normal population. Research also has found that some emotional disorders (such as manic depression) occur more often in families of a child with autism.

At least one group of researchers has found a link between an abnormal gene and autism. The gene may be just one of 3-5 or more genes that interact in some way to cause the condition. Scientists suspect that a faulty gene or genes might make a person more likely to develop autism when there are also other factors present, such as a chemical imbalance, viruses or chemicals, or a lack of oxygen at birth.

Other potential causes of autism are environmental toxins, including pesticides and heavy metals such as mercury. Heavy metals are certainly more commonly encountered in the environment now than they were in the past. It may be that people with autism or those at higher risk for developing it are more sensitive than others to these toxins.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. However, by late adolescence, pruning eliminates about half of these cortical synapses. In addition, many genes linked to autism are known to affect the development or function of brain synapses. The study also found that the brain cells from individuals with autism were filled with damaged parts and deficient in signs of a normal breakdown pathway called “autophagy.” Tang et al. 2014.

Thus, one embodiment of the disclosure is directed to methods of treating autism comprising administering a therapeutically effective amount of an aminosterol composition according to the disclosure. In one embodiment, treatment results in improvement in one or more characteristics of autism. Such characteristics can be, for example, communication skills, social interaction, sensory sensitivity, and behavior. Improvement can be measured using any clinically recognized tool or assessment.

For example, the methods of the disclosure may show an improvement in one or more characteristics of autism, such as behavior, communication, mood, etc., as measured by a medically recognized scale. The improvement may be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The medically recognized scale may be selected from, for example, Childhood Autism Rating Scale (CARS), Autism Spectrum Rating Scales, or The Michigan Autism Spectrum Questionnaire.

### (3) Cerebral or General Ischemic Disorder and/or a Related Symptom

In one embodiment, a method of treating, preventing, and/or slowing the onset or progression of a cerebral or general ischemic disorder and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

In one embodiment, the cerebral or general ischemic disorder is selected from microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, or pulmonary edema.

For example, the methods of the disclosure may show an improvement in one or more characteristics of the cerebral or general ischemic disorder as measured by a medically recognized scale. The improvement may be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Medically recognized scales or techniques to measure improvement include, for example, cholesterol test, high-sensitivity C-reactive protein test, lipoprotein (a), plasma ceramides, natriuretic peptides, low density lipoprotein cholesterol, high density lipoprotein cholesterol, triglycerides, electrocardiogram (EKG), Holter monitor, stress test, echocardiogram, positron emission tomography (PET), thallium scans, myocardial perfusion scans, implantable loop recorder, tilt table test, electrophysiology study, coronary angiogram, magnetic resonance imaging, magnetic resonance angiography, cardiac CT scan, and event recorder.

(i) Erectile Dysfunction

In one embodiment, a method of treating, preventing, and/or slowing the onset or progression of erectile dysfunction (ED) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Erectile dysfunction can be a sign of a physical or psychological condition. It can cause stress, relationship strain, and low self-confidence. The main symptom is a man's inability to get or keep an erection firm enough for sexual intercourse. ED can occur manifest through different mechanisms. Based on its mechanism, ED can be classified as psychogenic, neurogenic (failure to initiate erection), artereogenic (failure of the penis to fill with blood), cavernosal (failure of vascular system to retain blood in penis once filled) (Dean et al. 2005).

Psychogenic ED can arise because sexual behavior and penile erection are controlled by the hypothalamus, the limbic system, and the cerebral cortex. Therefore, stimulatory or inhibitory messages can be relayed to the spinal erection centers to facilitate or inhibit erection. Two possible mechanisms have been proposed to explain the inhibition of erection in psychogenic dysfunction: direct inhibition of the spinal erection center by the brain as an exaggeration of the normal suprasacral inhibition and excessive sympathetic outflow or elevated peripheral catecholamine levels, which may increase penile smooth muscle tone to prevent the relaxation necessary for erection.

Neurogenic ED may arise as a result of pathology in the brain. The medial preoptic area, the paraventricular nucleus, and the hippocampus have been regarded as important integration centers for sexual drive and penile erection. Pathologic processes in these regions, in conditions such as Parkinson's disease, stroke, encephalitis, or temporal lobe epilepsy, are often associated with ED. Other lesions in the brain noted to be associated with ED are tumors, dementias, Alzheimer's disease, Shy-Drager (multiple system atrophy), syndrome, and trauma.

Many neurodiseases causing ED such as PD are suspected to correlate with the formation of toxic αS aggregates within the enteric nervous system (ENS) (Braak et al. 2003 (a) and (b)). ED has been reported to affect in the range of 60-79% of men having PD, while the prevalence of ED in non-Parkinson men is only about 37.5% (Papatsoris, 2006). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008), and thereby positively impact ED associated with abnormal αS pathology.

Typically, ED manifests several years after PD has been established in the patient. Neurodegenerative conditions such as PD may cause damage to brain centers responsible for autonomic processing. It is believed that aminosterols capable of treating or preventing neurodegeneration in PD, may prevent or treat the degeneration of neuronal structure that governs erection either directly or indirectly via the regulation of hormones.

It is known that central dopamine is a key neurotransmitter in the control of sexual function including erection (Giuliano et al 2001). It is thought that dopamine deficiency may be responsible for erectile dysfunction often observed in PD patients (Palma et al 2014). In patients with PD, αS-related pathology develops in serotonergic and cholinergic neurons in parallel with that seen in the nigral dopamine neurons. Thus, regulation of αS may play a role in ED in PD via dopaminergic dysfunction.

In one embodiment, the method results in a decrease in the number of instances in which the subject cannot attain erection, and the decrease in number of instances in which the subject cannot attain erection comprises a reduction in number of instances in which the subject cannot attain erection over a defined period of time. In another aspect, the method results in a decreased severity of ED over a defined period of time, wherein the decreased severity of ED is measured by a medically recognized technique selected from the group consisting of bone-pressed erect length (BPEL) measurement, girth measurement, Erection Hardness Scale (EHS), and International Index of Erectile Function (IIEF).

(ii) Blood Pressure

In one embodiment, a method of treating, preventing, and/or slowing the onset or progression of high blood pressure (HBP) or low blood pressure (LBP) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

High blood pressure (HBP), also referred to as hypertension, is a long-term medical condition in which the blood pressure in the arteries is persistently elevated. Long-term high blood pressure, is a major risk factor for coronary artery disease, stroke, heart failure, atrial fibrillation, peripheral vascular disease, vision loss, chronic kidney disease, and dementia. HBP may be characterized as (a) a systolic blood pressure (BP)≥120 and a diastolic BP<80; or (b) a systolic blood pressure (BP)≥130 or a diastolic BP≥80; while low blood pressure (LBP) may be characterized as (a) a systolic blood pressure≤80; or (b) a diastolic blood pressure≤50.

Low blood pressure (LBP), also referred to as hypotension, is generally classified as a systolic blood pressure of less than 90 millimeters of mercury (mm Hg) or diastolic of less than 60 mm Hg. Primary symptoms include lightheadedness, vertigo and fainting. Severely low blood pressure can deprive the brain and other vital organs of oxygen and nutrients, leading to a life-threatening condition called shock. For some people who exercise and are in top physical condition, low blood pressure is a sign of good health and fitness. For many people, excessively low blood pressure can cause dizziness and fainting or indicate serious heart, endocrine or neurological disorders.

Blood Pressure (BP) and αS pathology: Many neurodiseases causing HBP or LBP, such as PD, are suspected to correlate with the formation of toxic αS aggregates within the enteric nervous system (ENS) (Braak et al. 2003 (a) and (b)). In a study of 11.55 million PD patient doctor visits in the US, the most commonly recorded comorbidity was hypertension, in 37.8% of visits (Lingala et al. 2017). Orthostatic hypotension (OH) is one of the commonly occurring nonmotor symptoms in patients with idiopathic Parkinson's disease (IPD) (Fereshtehnejad et al. 2014).

Studies suggest that a persistent hypertension can cause abnormal accumulation of phosphorylated αS in rats (Sato et al. 2014; Fukui et al. 2014). Also, mice genetically engineered to overexpress human αS showed differing cardiac responses to chemically induced hypotension compared to wildtype mice (Fleming et al. 2013).

One in five patients with PD are affected by orthostatic hypotension, which may manifest as a drop in blood pressure upon standing up. Elevated systolic blood pressure predicts worsening motor function among patients with Parkinson's disease (Lineback 2016). Neurodegenerative conditions such as PD may cause damage to brain centers responsible for autonomic processing, essential for regulation of blood pressure. It is believed that aminosterols capable of treating or preventing neurodegeneration in PD, may prevent or treat the degeneration of neuronal structure that governs regulation of blood pressure either directly or indirectly via the regulation of hormones.

The αS abnormalities typically found in PD are believed to be responsible for apparent catecholamine-deficits (dopamine is a catecholamine sharing metabolic pathways with other catecholamines) (Frisina et al., 2009). It is known that dopamine is a key neurotransmitter regulating blood pressure (Jose et al. 2003). Dopamine's actions on renal hemodynamics, epithelial transport and humoral agents such as aldosterone, catecholamines, endothelin, prolactin, pro-opiomelanocortin, renin and vasopressin place it in central homeostatic position for regulation of blood pressure. Dopamine also modulates fluid and sodium intake via actions in the central nervous system and gastrointestinal tract, and by regulation of cardiovascular centers that control the functions of the heart, arteries and veins. Abnormalities in dopamine production and receptor function accompany a high percentage of human essential hypertension and several forms of rodent genetic hypertension. In patients with PD, α-synuclein-related pathology develops in serotonergic and cholinergic neurons in parallel with that seen in the nigral dopamine neurons. Thus, regulation of αS may play a role in blood pressure dysregulation in PD via dopaminergic dysfunction.

Examples of conditions associated with abnormal αS pathology, and/or dopaminergic dysfunction, correlated with HBP or LBP include, but are not limited to, synucleinopathies, neurodiseases, psychological and/or behavior disorders, cerebral and general ischemic disorders, examples of which are described herein.

In one embodiment, in a subject having HBP, the method lowers the systolic and/or diastolic blood pressure by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In one embodiment, in a subject having LBP, the method raises the systolic and/or diastolic blood pressure by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In one embodiments, the clinically recognized scale or tool is selected from the group consisting of sphygmomanometry, arterial penetration, palpitation, asuculatoration, oscillometry, continuous noninvasive arterial pressure (CNAP), pulse wave velocity, and ambulatory monitoring.

(iii) Cardiac conduction defects

In one embodiment, a method of treating, preventing, and/or slowing the onset or progression of cardiac conduction defects (CCDs) and/or a related symptom in a subject in need is provided, comprising administering to the subject a therapeutically effective amount of at least one aminosterol disclosed herein (e.g., Compound VI), or pharmaceutically acceptable salt, solvate, prodrug, or derivative thereof.

Cardiac conduction defects (CCDs) involve an aberration in how electrical impulses travel to and through the heart. The cardiac conduction system transmits electrical signals generated usually by the sinoatrial node to cause contraction of cardiac muscle. Cardiac conduction defect (CCD) is a serious and potentially life-threatening disorder. It belongs to a group of pathologies with an alteration of cardiac conduction through the atrioventricular (AV) node, the His-Purkinje system with right or left bundle branch block, and widening of QRS complexes in the electrocardiogram (EKG). Originally, CCD was considered a structural disease of the heart with anatomic changes in the conduction system underlying abnormal impulse propagation. In a substantial number of cases, however, conduction disturbances are found to occur in the absence of anatomical abnormalities. In these cases functional rather than structural alterations appear to underlie conduction disturbances. These functional defects are called “primary electrical disease of the heart.” The pathophysiological mechanisms underlying CCD are diverse, but the most frequent form of CCD is a degenerative form also called Lenegre-Lev disease (idiopathic bilateral bundle branch fibrosis). Today Lenegre-Lev disease represents a major cause of pacemaker implantation in the world.

Bundle branch block is a common type of CCD. Normally, electrical impulses travel down the right and left branches of the ventricles at the same speed. This allows both ventricles to contract simultaneously. When there is a “block” in one of the branches, electrical signals have to take a different path through the ventricle. This detour means that one ventricle contracts a fraction of a second slower than the other, causing an arrhythmia. A person with bundle branch block may experience no symptoms, especially in the absence of any other problems. An electrocardiogram (EKG or ECG) reveals bundle branch block when it measures the heart's electrical impulses.

Another common CCD is known as heart block. In cases of heart block, the electrical signals that progress from the heart's upper chambers (atria) to its lower chambers (ventricles) are impaired. When these signals transmit improperly, the heart beats irregularly. There are several degrees of heart block.

First-degree heart block occurs when the electrical impulse moves through the heart's AV node more slowly than normal. This usually results in a slower heart rate. The condition may cause dizziness or lightheadedness. Second-degree heart block occurs when electrical signals from the heart's upper chambers (atria) fail to reach the lower chambers (ventricles). This can result in skipped beats. Symptoms of second degree heart block include chest pain, fainting, palpitations, difficulty breathing, rapid breathing, nausea and fatigue. Third-degree, or complete, heart block means that electrical signals cannot pass at all from the heart's upper chambers (atria) to its lower chambers (ventricles). In the absence of electrical impulses from the sinoatrial node, the ventricles will still contract and pump blood, but at a slower rate than usual. Symptoms of third degree hear block are similar to those of second degree heart block. Heart conditions can cause third-degree heart block, as can certain medications in extreme cases. An injury to the heart's electrical conduction system during surgery can also cause third-degree heart block.

Long QT Syndrome, also called LQTS, is a CCD. In LQTS, the lower chambers of the heart (ventricles) take too long to contract and release. The gap of time needed to complete a cycle can be measured and compared to normal averages. The name for the condition comes from letters associated with the waveform created by the heart's electrical signals. The interval between the letters Q and T defines the action of the ventricles. Hence, long QT Syndrome means that time period is too long, even if by fractions of a second. An occasional prolonged QT interval can be precipitated by everyday circumstances, including: when startled by a noise, physical activity or exercise, intense emotion (such as fright, anger or pain). Some arrhythmias related to LQTS are potentially fatal and can cause cardiac arrest.

Other forms of CCD include atrioventricular (AV) blocks and wide or narrow QRS. Some forms of CCD are congenital, for example, Wolff-Parkinson White (WPW). In WPW, patients have an accessory pathway that communicates between the atria and the ventricles, in addition to the AV node. This accessory pathway is known as the bundle of Kent. This accessory pathway does not share the rate-slowing properties of the AV node, and may conduct electrical activity at a significantly higher rate than the AV node causing abnormally high heart rate.

In one embodiment, the CCD includes or results in (a) QT interval (QTc)≥440 ms; (b) syncope; (c) presence of delta wave in electrocardiogram (EKG); (d) pseudo-right bundle branch block in EKG; (e) ST elevations in V1-V3 in EKG; (f) a QRS complex>100 ms in EKG; (g) PR interval<120 ms in EKG; (h) heart rate above 100 beats per minute (BPM); (i) heart rate below 60 BPM; (j) PR interval>200 ms in EKG; (k) QRS not following a P wave in EKG; (1) no repeating relation between P wave and QRS complex in EKG; (m) differing atrial and ventricular rates; (n) QS or rS complex in lead V1 in EKG; (o) notched (‘M’-shaped) R wave in lead V6; (p) T wave discordance in EKG; (q) left axis deviation between −45° and −60° in EKG; (r) qR pattern (small q, tall R) in the lateral limb leads I and aVL in EKG; (s) rS pattern (small r, deep S) in the inferior leads II, III, and aVF in EKG; (t) delayed intrinsicoid deflection in lead aVL (>0.045 s) in EKG; (u) frontal plane axis between 900 and 1800 in EKG; (v) rS pattern in leads I and aVL in EKG; (w) qR pattern in leads III and aVF in EKG; (x) chest pain; (y) palpitations; (z) difficulty breathing; (aa) rapid breathing; (bb) nausea; (cc) fatigue; (dd) sleep problem, sleep disorder, or sleep disturbance; (ee) constipation; and (ff) cognitive impairment.

In one embodiment, progression or onset of CCD is slowed, halted, or reversed over a defined period of time following administration of the fixed escalated dose of the aminosterol or a salt or derivative thereof, as measured by a medically-recognized technique. In addition, the CCD can be positively impacted by the fixed escalated dose of the aminosterol or a salt or derivative thereof, as measured by a medically-recognized technique. The positive impact and/or progression of CCD can be measured quantitatively or qualitatively by one or more techniques selected from the group consisting of echocardiography, electrocardiography (ECG or EKG), magnetic resonance imaging (MRI), positron-emission tomography (PET); coronary catheterization, intravascular ultrasound, Holter monitoring, stress test, computed tomography angiography (CTA), and coronary CT calcium scan. In addition, the progression or onset of CCD can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof reverses dysfunction caused by the CCD and treats, prevents, improves, and/or resolves the symptom being evaluated. The improvement or resolution of the CCD symptom is measured using a clinically recognized scale or tool. In addition, the improvement in the CCD symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using, for example, the techniques described above.

C. Aminosterols for Treatment of Microbial Infections

The present disclosure is also directed to methods of treating a subject in need, wherein the subject has a microbial infection. The methods comprise administering to the subject a therapeutically effective amount of a composition comprising at least one aminosterol disclosed herein via inhalation, pulmonary, and/or nasal administration. In another aspect, the formulation can be administered by gargling to ensure drug application/exposure to the throat. Such an administration method can be particularly beneficial for treating respiratory infections, such as influenza and coronavirus.

The microbial infection can be, for example, a viral infection, fungal infection, protozoan infection, or bacterial infection. In addition, the microbial infection can be correlated with pneumonia and/or a lung infection.

Further, for the methods described herein, optionally the method can additionally comprise administering (a) an antimicrobial drug; and/or (b) an antiviral drug if the subject has a viral infection; and/or (c) an antibacterial drug if the subject has a bacterial infection; and/or (d) an antifungal drug if the subject has a fungal infection.

In one aspect of the methods, the subject is a human. In another aspect, the subject is at risk, or is a member of a patient population at risk, of developing the microbial infection to be treated.

The aminosterol may be in a lactate or dilactate salt form. In another aspect of the methods described herein, the lactate or dilactate salt of the aminosterol is administered at a very low dose of about 50 mg or less. In yet another aspect, the lactate or dilactate salt of the aminosterol is administered at a very low dose of about 45 mg or less, about 40 mg or less, about 35 mg or less, about 30 mg or less, about 25 mg or less, about 20 mg or less, about 15 mg or less, about 14 mg or less, about 14 mg or less, about 13 mg or less, about 12 mg or less, about 11 mg or less, or about 10 mg or less.

In yet another aspect, the lactate or dilactate salt of the aminosterol is administered at about 9.5 mg or less, about 9 mg or less, about 8.5 mg or less, about 8 mg or less, about 7.5 mg or less, about 7 mg or less, about 6.5 mg or less, about 6 mg or less, about 5.5 mg or less, about 5 mg or less, about 4.5 mg or less, about 4 mg or less, about 3.5 mg or less, or about 3 mg or less.

In one aspect, the present invention encompasses a method of treating a subject, wherein the subject is susceptible to or has an infection by one or more microorganisms.

In one aspect, the present invention encompasses a method of treating a subject, wherein the subject is susceptible to or has an infection by one or more gram-positive or gram-negative bacterial species. In another aspect, the present invention encompasses a method of treating a subject, wherein the subject is susceptible to or has an infection by one or more viruses. In yet another aspect, the present invention encompasses a method of treating a subject, wherein the subject is susceptible to or has an infection by one or more fungi. In one aspect, the present invention encompasses a method of treating a subject, wherein the subject is susceptible to or has an infection by one or more protozoan. In all of the methods described herein, the infection can be but is not limited to a pulmonary infection.

Compositions and methods of the present invention also find use in the treatment and/or prevention of a host of respiratory infections (e.g., respiratory infections of the upper respiratory tract (e.g., nose, ears, sinuses, and throat) and the lower respiratory tract (e.g., trachea, bronchial tubes, and lungs)).

(1) Microorganisms

The microbial infection can be caused for example by a virus, bacteria, fungus, or protozoan. The present invention is not limited by the type of microbe treated.

The viral infection to be treated or prevented can be caused by any virus, including but not limited to, “African Swine Fever Viruses,” Arbovirus, Adenoviridae, Arenaviridae, Arterivirus, Astroviridae, Baculoviridae, Bimaviridae, Birnaviridae, Bunyaviridae, Caliciviridae, Caulimoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g., Paramyxoviridae, Morbillivirus, Rhabdoviridae), Myoviridae, Orthomyxoviridae (e.g., Influenza A, Influenza B, and parainfluenza), Papiloma virus, Papovaviridae, Paramyxoviridae, Prions, Parvoviridae, Phycodnaviridae, Picomaviridae (e.g. Rhinovirus, Poliovirus), Poxviridae (such as Smallpox or Vaccinia), Potyviridae, Reoviridae (e.g., Rotavirus), Retroviridae (HTLV-I, HTLV-II, Lentivirus), Rhabdoviridae, Tectiviridae, Togaviridae (e.g., Rubivirus), or any combination thereof. In another embodiment of the invention, the viral infection is caused by a virus selected from the group consisting of herpes, pox, papilloma, corona, influenza, hepatitis, sendai, sindbis, vaccinia viruses, west nile, hanta, or viruses which cause the common cold. In another embodiment of the invention, the condition to be treated is selected from the group consisting of AIDS, viral meningitis, Dengue, EBV, hepatitis, and any combination thereof.

In one aspect, the viral infection is caused by a coronavirus. For example, the coronavirus can be selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus. In one embodiment, the coronavirus is SARS-CoV-2.

The terms “bacteria” and “bacterium” refer to all prokaryotic organisms, including those within all of the phyla in the Kingdom Procaryotae. All forms of bacteria are included within this definition including cocci, bacilli, spirochetes, spheroplasts, protoplasts, etc. Also included within this term are prokaryotic organisms that are Gram-negative or Gram-positive. Examples of bacteria include, but are not limited to, bacterial cells of a genus of bacteria selected from the group comprising Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

The term “fungi” is used in reference to eukaryotic organisms such as molds and yeasts, including dimorphic fungi.

Compositions and methods of the present invention can be utilized to treat (e.g., kill and/or inhibit growth of) organisms capable of forming biofilms including, but not limited to, dermatophytes (e.g, Microsporum species such as Microsporum canis, Trichophyton species such as Trichophyton rubrum and Trichophyton mentagrophytes), yeasts (e.g., Candida albicans, Candidaparapsilosis, Candida glabrata, Candida tropicalis, and other Candida species including drug resistant Candida species), Epidermophytonfloccosum, Malasseziafuurfur (Pityropsporon orbiculare, Pityropsporon ovale) Cryptococcus neoformans, Aspergillusfumigatus and other Aspergillus species, Zygomycetes (Rizopus, Mucor), hyalohyphomycosis (Fusarium species), Paracoccidioides brasiliensis, Blastmyces dermatitides, Histoplasma capsulatum, Coccidiodes immitis, Sporothrix schenckii, and Blastomyces.

The present invention also provides compositions and methods for treating (e.g., killing and/or inhibiting growth of) microorganisms that heretofore display resistance to a broad spectrum of antibiotics (e.g., species of the genus Acinetobacter).

Acinetobacter species are generally considered nonpathogenic to healthy individuals. However, several species persist in hospital environments and cause severe, life-threatening infections in compromised patients. The spectrum of antibiotic resistances of these organisms together with their survival capabilities make them a threat to hospitals as documented by recurring outbreaks both in highly developed countries and elsewhere. Infections occur in immunocompromised individuals, and the strain A. baumannii is the second most commonly isolated nonfermenting bacteria in human specimens. Acinetobacter is frequently isolated in nosocomial infections and is especially prevalent in intensive care units, where both sporadic cases as well as epidemic and endemic occurrence is common. A. baumannii is a frequent cause of nosocomial pneumonia, especially of late-onset ventilator associated pneumonia. It can cause various other infections including skin and wound infections, bacteremia, and meningitis. A. lwoffi is also causative of meningitis. A. baumannii can survive on the human skin or dry surfaces for weeks.

Acinetobacter species are innately resistant to many classes of antibiotics, including penicillin, chloramphenicol, and often aminoglycosides. Thus, in some embodiments, compositions and methods of the present invention are utilized to treat (e.g., kill and/or inhibit growth of) bacteria of the Acinetobacter species (e.g., individually or in combination with other treatments (e.g., carbapenems, polymyxin B, and/or sulbactam)).

(2) Respiratory Infections/Diseases

In one aspect of the disclosure, encompassed are methods of treating pulmonary and/or respiratory infections with an inhaled aminosterol composition. “Respiratory infection” and “pulmonary infection” refer to a microbial infection (e.g., bacterial, viral, fungal, etc.) of the respiratory tract. In humans, the respiratory tract comprises the upper respiratory tract (e.g., nose, throat or pharynx, and larynx); the airways (e.g., voice box or larynx, windpipe or trachea, and bronchi); and the lungs (e.g., bronchi, bronchioles, alveolar ducts, alveolar sacs, and alveoli).

“Respiratory disease”, “pulmonary disease,” “respiratory disorder”, “pulmonary disorder,” “respiratory condition”, “pulmonary condition,” “pulmonary syndrome,” and “respiratory syndrome” refer to any one of several ailments that involve inflammation and affect a component of the respiratory system including especially the trachea, bronchi and lungs. In another aspect of the disclosure, encompassed are methods of treating such conditions comprising administration of an inhaled lactate or dilactate aminosterol composition. Examples of such ailments include acute alveolar disease, obstructive respiratory disease (e.g., asthma; bronchitis; and chronic obstructive pulmonary disease, referred to as COPD), upper airway disease (e.g., such as otitis media, and rhinitis/sinusitis), cystic fibrosis (CF), insterstitial lung disease, allergy, and respiratory infection (e.g., pneumonia, pneyumocystis carinii, and respiratory syncitial virus (RSV)).

Specific examples of acute alveolar disease include acute lung injury (ALI), acute respiratory distress syndrome (ARDS), meconium aspiration syndrome (MAS) and respiratory distress syndrome (RDS). ALI is associated with conditions that either directly or indirectly injure the air sacs of the lung, the alveoli. ALI is a syndrome of inflammation and increased permeability of the lungs with an associated breakdown of the lungs' surfactant layer. The most serious manifestation of ALI is ARDS. Among the causes of ALI are complications typically associated with certain major surgeries, mechanical ventilator induced lung injury (often referred to as VILI), smoke inhalation, pneumonia, and sepsis.

Cystic fibrosis (CF) is a life-threatening disorder that causes severe lung damage due to a defective transmembrane protein called CFTR responsible for the balance of electrolytes. Thick mucus forms plugging the tubes, ducts and passageways in the lungs. This environment is ideal for opportunistic bacteria to establish biofilm communities, leading to respiratory infections. Systemically-administered antibiotics can decrease the frequency and severity of exacerbations; however, the bacteria are never be completely eradicated from the airways and the lungs. Nebulized antibiotics are used, but resistance emergence and/or colonization of different resistant species is a major concern. Cystic fibrosis (CF) results in the functional impairment of innate respiratory defense mechanisms, providing an environment for colonization of pathogenic bacterial species such as Staphylococcus aureus and Haemophilus influenzae, and a number of opportunistic species such as Pseudomonas aeruginosa, Achromobacter xylosoxidans, Stenotrophomonas maltophilia, Ralstonia spp., Pandoraea spp., and the Burkholderia cepacia complex (Bcc) species. The Bcc comprises a group of at least 17 phylogenetically related saprophytic gram-negative bacilli, most of which can form biofilm. They are particularly difficult to treat and are associated with increased rates of morbidity and mortality in CF patients. They also are among the most antimicrobial-resistant bacterial species encountered in human infections. Once established, the infection and associated inflammation are rarely eliminated, resulting in progressive lung disease ending in pulmonary failure and death.

(i) Coronavirus

Squalamine has been shown to inhibit a specific isoform of the sodium-hydrogen exchanger (“NHE-3”), a protein that plays a role in numerous cellular processes that involve the control of intracellular hydrogen ions (Akhter, Nath et al. 1999). As a consequence of this activity, it was proposed that squalamine might find utility in treating diseases, including viral infections, where NHE3 played a critical role, and where its inhibition (by squalamine) could be effected (see e.g., U.S. Pat. No. 6,962,909). It has been proposed that squalamine could be used to treat viral infections should it be known that a specific virus infected a target cell expressing an NHE sensitive to inhibition (NHE-3 in the case of squalamine), and that the specific NHE played a critical role in the cellular homeostasis of that cell type, and that the virus in question naturally infected that cell type in the course of a disease process (U.S. Pat. No. 6,962,909). To date, however, no example of an NHE-3 dependent viral infection has been reported in the literature, nor has any known NHE-3 inhibitor been shown to exhibit antiviral activity in an animal, including squalamine. Furthermore the viruses demonstrated to be inactivated in vitro by squalamine, namely HIV and HSV (WO96/08270) are now known to infect cells via a pathway that is “pH independent”, in the sense that inhibitors of pH homeostasis do not influence infectivity (Pelkmans and Helenius 2003).

To date, no published data describe or support the efficacy of squalamine in treating or preventing a systemic viral infection in an animal. It has been reported in a patent application that squalamine could inhibit the infectivity of HIV and HSV in tissue culture (WO96/08270). However, it was not reported at that time, nor until the invention disclosed herein, that squalamine could exhibit antiviral activity when administered systemically to an animal. In the experiments described in WO96/08270, squalamine was conceived as a component of a topical agent to be used as a “chemical condom”, acting as a microbicide, and capable of rapidly inactivating HIV or HSV on contact by disrupting the outermost membranous envelopes of the viruses. Thus, the antiviral properties of squalamine observed in vitro were believed to result from direct disruption of the viral membrane, via a mechanism analogous to that proposed for its antibacterial activity. The potential use of squalamine for the topical prevention of sexually transmitted diseases such as HIV, Herpes simplex, and Neisseria gonorrhea was presented at the 1995 ICAAC conference (MacDonald 1995). Thus, squalamine was proposed to have utility as an advanced form of “disinfectant,” to be applied to a mucosal surface in some formulation and thereby prevent viable virus from gaining access to the epithelial surfaces of the genitourinary tract. It is thus envisaged that aminosterols may be useful in the treatment and prevention of other virus types, for example, coronaviruses.

In one aspect, a method of treating or preventing an infection by a coronavirus in a subject is provided, comprising administering a therapeutically effective amount of the aminosterol compound of any embodiment herein or the composition of any embodiment herein to the subject.

In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus.

In some embodiments, the coronavirus is encoded by a polynucleotide comprising the sequence of SARS-CoV-2, or a polynucleotide having at least 80% sequence identity to the polynucleotide comprising the sequence of SARS-CoV-2. In some embodiments, the coronavirus comprises or is characteristic of human coronavirus 229E, human coronavirus OC43, SARS-CoV, HCoV NL63, HKU1, MERS-CoV, or SARS-CoV-2. In some embodiments, the coronavirus comprises or is characteristic of SARS-CoV-2.

In some embodiments, the subject is deemed at risk for severe illness and/or serious complications from the infection. In some embodiments, the subject is about age 50 or older, about age 55 or older, about age 60 or older, or about age 65 or older. In some embodiments, the subject suffers from one or more pre-existing conditions selected from the group consisting of diabetes, asthma, a respiratory disorder, high blood pressure, and heart disease. In some embodiments, the subject is immunocompromised. In some embodiments, the subject is immunocompromised due to AIDS, cancer, a cancer treatment, hepatitis, an auto-immune disease, steroid receiving, immunosenescence, or any combination thereof.

In some embodiments, administration increases the chance of survival for the subject following exposure to a coronavirus. In some embodiments, the chance of survival is increased by about 10%, about 20%, about 30%, about 40%, about 50%, about 60%, about 70%, about 80%, about 90%, or about 100%, as measured using any clinically recognized technique.

In some embodiments, the subject is exposed to or is anticipated to be exposed to an individual who is contagious for a coronavirus. In some embodiments, the individual who is contagious for a coronavirus has one or more symptoms selected from the group consisting of fever, cough, shortness of breath, diarrhea, sneezing, runny nose, and sore throat. In some embodiments, the subject is a healthcare worker, aged 60 years or older, frequent traveler, military personnel, caregiver, or a subject with a preexisting condition that results in increased risk of mortality with infection.

In some embodiments, the method further comprises administering one or more antiviral drugs. In some embodiments, the one or more antiviral drugs are selected from the group consisting of chloroquine, hydroxychloroquine, darunavir, galidesivir, interferon beta, lopinavir, ritonavir, remdesivir, and triazavirin.

In some embodiments, administration reduces the risk of transmission of coronavirus. In some embodiments, the reduction in risk of transmission is by about 10%, about 20%, about 30%, about 40%, about 50%, about 60%, about 70%, about 80%, about 90%, or about 100%, as measured using any clinically recognized technique. In some embodiments, the medically recognized technique comprises PCR (polymerase chain reaction), a test of Table 1A below, or immunoassay.

Cells that are rich in a cell-surface receptor called angiotensin-converting enzyme 2 (ACE2) are more readily invaded by coronaviruses because the virus requires that receptor (ACE2) to enter a cell. In one aspect a method of treating and or preventing infection by a coronavirus (i.e., SARS-CoV-2) in a subject is provided, comprising administering to an ACE2-rich tissue of the subject an aminosterol disclosed herein. ACE-2 rich tissues may include lung, alveoli, renal, neural cortex, brain stem, and digestive tract tissue.

(ii) COVID-19

Coronavirus disease 2019 (COVID-19) is an infectious disease caused by severe acute respiratory syndrome coronavirus 2 (SARS-CoV-2). The disease was first identified in December 2019 in Wuhan, the capital of China's Hubei province, and has since spread globally, resulting in the ongoing 2019-20 coronavirus pandemic. Symptoms include fever, cough, shortness of breath, diarrhea, sneezing, runny nose, blood clotting, low blood O₂, and sore throat.

In one aspect, a method of treating a subject in need having COVID-19 is provided, comprising administering a therapeutically effective amount of an aminosterol herein. In some embodiments, of the present methods, the subject has been diagnosed with and/or tested positive for COVID-19. COVID-19 tests include any commercially available test known in the art, for example those in Table 1A below, or a positive test result may be determined using, for example, polymerase chain reaction (PCR) or immunoassay. In one embodiment, the subject has been diagnosed with COVID-19, is at risk of contracting COVID-19, or is suspected of suffering from COVID-19.

TABLE 1A
Commercially available tests for SARS-CoV-2 and COVID-19

| Company Name | Test Name |
|---|---|
| 3D Medicines | SARS-CoV-2 and Influenza A & B RT-qPCR Detection Kit |
| Abbott | SARS-CoV-2 IgG test |
| Abbott | ID Now COVID-19 |
| Abbott | Abbott RealTime SARS-CoV-2 EUA test |
| Anatolia Geneworks | Bosphore Novel Coronavirus (2019-nCoV) Detection Kit |
| ARUP Laboratories | COVID-19 |
| A*STAR, Tan Tock Seng Hospital of | A*STAR Fortitude 2.0 |
| Singapore | |
| Assure Tech | COVID-19 IgG/IgM Rapid Test Device |
| Atila BioSystems | iAMP COVID-19 Detection Kit |
| AusDiagnostics | AusDiagnostics SARS-CoV-2, influenza, RSV panel |
| Autobio Diagnostics | Anti-SARS-CoV-2 Rapid Test |
| Avellino Lab | Avellino SARS-CoV-2/COVID-19 (AvellinoCoV2) |
| Bako Diagnostics | BakoDx SARS-CoV-2 RNA test |
| Baptist Hospital Miami | COVID-19 RT-PCR Test |
| Pathology/Laboratory Medicine Lab | |
| Becton Dickinson | BD SARS-CoV-2 Reagents for BD MAX System |
| Becton Dickinson, BioGx | BioGX SARS-CoV-2 Reagents for the BD MAX System |
| Beijing Decombio Biotechnology | Novel Coronavirus IgM/IgG Combo Rapid Test-Cassette |
| Beijing Diagreat Biotechnologies | 2019-nCoV IgG, IgM Antibody Determination Kits |
| | 2019-nCoV IgG/IgM Antibody Rapid Test Kit |
| Beijing Kewei Clinical Diagnostic | Genonto RapidTest10 COVID-19 IgG/IgM Antibody Rapid Test Kit |
| Reagent | |
| Beijing O&D Biotech | Coronavirus disease (COVID-19) Total Antibody Rapid Test |
| | (Colloidal Gold) |
| Beroni Group | SARS-CoV-2 IgG/IgM Antibody Detection Kit |
| BGI | Real-Time Fluorescent RT-PCR kit for detecting SARS-2019-nCoV |
| Biodesix | SARS-CoV-2 Droplet Digital PCR (ddPCR) test |
| Biolidics | 2019-nCoV IgG/IgM Detection Kit (Colloidal Gold) |
| BioMedomics | COVID-19 IgM-IgG Rapid Test |
| BioMérieux | SARS-COV-2 R-GENE test |
| BioMérieux/BioFire Defense | BioFire COVID-19 test |
| Bioneer | AccuPower COVID-19 Real-Time RT-PCR Kit, AccuPower SARS- |
| | CoV-2 Real-Time RT-PCR Kit |
| Bio-Rad Laboratories | SARS-CoV-2 Total Ab test |
| BioReference Laboratories | Novel Coronavirus COVID-19 |
| Boston Children's Hospital Infections | Childrens-Altona-SARS-CoV-2 assay |
| Diseases Diagnostic Laboratory (IDDL) | |
| BTNX | Rapid Response COVID-19 IgG/IgM Test Cassette |
| Cellex | qSARS-CoV-2 IgG/IgM Rapid Test |
| Centers for Disease Control and | CDC 2019-Novel Coronavirus (2019-nCoV) Real-Time RT-PCR |
| Prevention (performed at qualified high- | Diagnostic Panel (CDC) |
| complexity CLIA laboratories designated | |
| by CDC) | |
| Cepheid | Xpert Xpress SARS-CoV-2 test |
| CerTest BioTec | ViaSure SARS-CoV-2 Real Time PCR Detection Kit |
| Chembio Diagnostics | DDP COVID-19 IgM/IgG System |
| Children's Hospital of Philadelphia | SARS-CoV-2 RT-PCR test |
| Infectious Disease Diagnostics | |
| Laboratory | |
| ChromaCode | HDPCR SARS-CoV-2 real-time PCR assay |
| CirrusDx Laboratories | CirrusDx SARS-CoV-2 Assay |
| Co-Diagnostics | Logix Smart Coronavirus Disease 2019 (COVID-19) Kit |
| Core Technology | CoreTest COVID-19 IgM/IgG Ab Test |
| Credo Diagnostics Biomedical | VitaPCR SARS-CoV2 Assay |
| DiaCarta | QuantiVirus SARS-CoV-2 test kit |
| Diagnostic Solutions Laboratory | COVID-19 Assay |
| DiaSorin Molecular | Simplexa COVID-19 Direct |
| Diatherix Eurofins | COVID-19 Panel |
| Diazyme Laboratories | Diazyme DZ-LITE SARS-CoV-2 IgG, IgM CLIA Kits |
| Eachy Biopharmaceuticals | AccuRapid SARS-CoV-2 IgM/IgG Test Kit (Lateral Flow |
| | Immunoassay) |
| Euroimmun/PerkinElmer | EuroRealTime SARS-CoV-2 |
| Euroimmun/PerkinElmer | Anti-SARS-CoV-2 ELISAs (IgA and IgG) |
| Exact Sciences | SARS-CoV-2 Test |
| Fosun Pharma USA | Fosun COVID-19 RT-PCR Detection Kit |
| Fulgent Genetics/MedScan Laboratory | COVID-19 |
| Genetic Signatures | Easy Screen SARS-CoV-2 detection kit |
| Genetron | Detection Kit for Novel Coronavirus (SARS-CoV-2) RNA (PCR- |
| | Fluorescence Probing) |
| GenMark Diagnostics | ePlex SARS-CoV-2 Test |
| Genomica/PharmMar Group | 2 kits: qCOVID-19, CLART COVID-19 |
| GenoSensor | GS COVID-19 RT-PCR Kit |
| Gnomegen | Gnomegen COVID-19 RT-Digital PCR Detection Kit |
| Gold Standard Diagnostics | SARS-CoV-2 IgG, IgM, IgA assays |
| Guangzhou Wondfo Biotech | SARS-CoV-2 Antibody Test |
| Hackensack University Medical Center | CDI Enhanced COVID-19 Test |
| (HUMC) Molecular Pathology | |
| Laboratory | |
| Hangzhou AllTest Biotech | AllTest 2019-nCoV IgG/IgM Rapid Test Cassette, AllTest COVID |
| | IgG/IgM Rapid Test Dipstick |
| Hangzhou Biotest Biotech | COVID-19 IgG/IgM Rapid Test Cassette |
| Hangzhou Clongene Biotech | Clungene COVID-19 IgM/IgG Rapid Test Cassette |
| Hangzhou Testsealabs Biotechnology | One Step SARS-CoV2 (COVID-19) IgG/IgM Test |
| Healgen Scientific | COVID-19 IgG/IgM Rapid Test Cassette(Whole |
| | Blood/Serum/Plasma) |
| Hologic | Panther Fusion SARS-CoV-2 assay |
| InBios International | Smart Detect SARS-CoV-2 rRT-PCR Kit |
| Innovita (Tangshan) Biological | 2019-nCoV Ab Test (Colloidal Gold) |
| Technology | |
| Integrated DNA Technologies/Danaher | IDT 2019-novel coronavirus kit |
| Integrity Laboratories | SARS-CoV-2 Assay |
| Ipsum Diagnostics | COV-19 IDx assay |
| Jiangsu Macro & Micro-Test Med-Tech | SARS-CoV-2 IgM/IgG Rapid Assay Kit (Colloidal Gold) |
| JN Medsys | ProTect Covid-19 kit |
| Kogene Biotech | 2019 Novel Coronavirus Real-time PCR Kit |
| KorvaLabs | Curative-Korva SARS-Cov-2 Assay |
| Laboratory Corporation of America | LabCorp 2019 Novel Coronavirus test |
| LGC, Biosearch Technologies | 2019-nCoV CDC-qualified Probe and Primer Kits for SARS-CoV-2 |
| Lifeassay Diagnostics | Test-it COVID-19 IgM/IgG Lateral Flow Assay |
| Luminex | ARIES SARS-CoV-2 Assay |
| Luminex | NxTAGCoV Extended Panel Assay |
| Maccura Biotechnology | SARS-CoV-2 Fluorescent PCR Kit |
| Massachusetts General Hospital | MGH COVID-19 qPCR assay |
| Medical Systems Biotechnology | Coronavirus Disease 2019 Antibody (IgM/IgG) Combined Test Kit |
| Mesa Biotech | Accula SARS-CoV-2 Test |
| Mount Sinai Labs | COVID-19 ELISA IgG Antibody Test |
| Nanjing Liming Bio-products | SARS-CoV-2 IgM/IgG Antibody Rapid Test Kit |
| NanoResearch | NanoMedicina SARS-CoV-2 IgM/IgG Antibody Rapid Test |
| Nantong Diagnos Biotechnology | (2019-nCoV) New coronavirus Antibody Test (Colloidal Gold) |
| NeuMoDx Molecular | NeuMoDx SARS-CoV-2 Assay |
| Nirmidas Biotech | COVID-19 (SARS-CoV-2) IgM/IgG Antibody Detection Kit |
| Northwestern Medicine Diagnostic | SARS-Cov-2 Assay |
| Molecular Laboratory | |
| Novacyt/Primerdesign | COVID-19 Genesig Real-Time PCR assay |
| NY State Department of Health | New York SARS-CoV-2 Real-time Reverse Transcriptase (RT)- |
| (performed at Wadsworth Center and | PCR Diagnostic Panel |
| New York City Department of Health and | |
| Mental Hygiene, Public Health | |
| Laboratories) | |
| Orig3n | Orig3n 2019 Novel Coronavirus (COVID-19) Test |
| Ortho Clinical Diagnostics | Vitros Immunodiagnostic Products Anti-SARS-CoV-2 Total |
| | Reagent Pack and Calibrators |
| Osang Healthcare | GeneFinder COVID-19 Plus RealAmp Kit |
| PathoFinder | RealAccurate Quadruplex Corona-plus PCR Kit |
| PCL | COVID19 IgG/IgM Rapid Gold |
| PerkinElmer | PerkinElmer New Coronavirus Nucleic Acid Detection Kit |
| Phamatech | COVID19 IgG/IgM Rapid Test |
| Promedical | COVID-19 Rapid Test, Wondfo SARS-CoV-2 Antibody Test |
| | (Lateral Flow Method) |
| Qiagen | QiaStat-Dx Respiratory SARS-CoV-2 Panel |
| Quest Diagnostics | Coronavirus Disease 2019 (COVID-19) Test |
| Quidel | Lyra SARS-CoV-2 Assay |
| Rendu Biotechnology | 2019-nCoV detection kit |
| Roche | Cobas SARS-CoV-2 Test |
| Rutgers University Clinical Genomics | ThermoFisher - Applied Biosystems TaqPath COVID-19 Combo Kit |
| Laboratory | |
| ScienCell Research Laboratories | ScienCell SARS-CoV-2 Coronavirus Real-time RT-PCR (RT- |
| | qPCR) Detection Kit |
| SD Biosensor | Standard Q COVID-19 IgM/IgG Duo |
| Seegene | Allplex 2019-nCoV Assay |
| Sentinel Diagnostics | STAT-NAT COVID-19 HK kit, B kit |
| Shanghai Fosun Long March Medical | novel coronavirus nucleic acid detection kit |
| Science/Shanghai Fosun Pharmaceutical | |
| Shenzhen Landwind Medical | COVID-19 IgG/IgM Rapid Test Device |
| Snibe Diagnostics | Maglumi 2019-nCoV (SARS-CoV-2) IgM/IgG kits |
| SolGent | DiaPlexQ Novel Coronavirus (2019-nCoV) Detection kit |
| Specialty Diagnostic (SDI) Laboratories | SDI SARS-CoV-2 Assay |
| Stanford Health Care Clinical Virology | SARS-CoV-2 PCR Assay |
| Laboratory | |
| SureScreen Diagnostics | SureScreen COVID19 IgM/IgG Rapid Test Cassette |
| Suzhou Kangheshun Medical Technology | SARS-CoV-2 IgG/IgM Rapid Test Cassette |
| Systaaq Diagnostic Products | 2019-Novel Coronavirus (COVID-19) Real Time PCR Kit |
| Telepoint Medical Services | SARS-CoV-2 IgG/IgM Rapid Qualitative Test |
| Thermo Fisher Scientific | TaqPath COVID-19 Combo Kit, RT-PCR CE-IVD Kit |
| Tianjin Beroni Biotechnology | SARS-CoV-2 IgG/IgM Antibody Detection Kit |
| TIB Molbiol Syntheselabor | Sarbecovirus E-gene |
| Trax Management Services | Phoenix Dx 2019-CoV |
| United Biomedical | UBI SARS-CoV-2 ELISA |
| University of North Carolina Medical | UNC Health SARS-CoV-2 real-time RT-PCR test |
| Center | |
| Vela Diagnostics | ViroKey SARS-CoV-2 RT-PCR Test |
| Viracor Eurofins | Viracor SARS-CoV-2 assay |
| Vision Medicals | SARS-CoV-2 Clinical Sequencing assay |
| VivaChek Biotech (Hangzhou) | VivaDiag COVID-19 IgM/IgG Rapid Test |
| Yale New Haven Hospital Clinical | SARS-CoV-2 PCR test |
| Virology Laboratory | |
| YD Diagnostics | MolecuTech Real-Time COVID-19 |
| Zhejiang Orient Gene Biotech | COVID-19 IgG/IgM Rapid Test Cassette |
| Zhengzhou Fortune Bioscience | IgG/IgM Antibody Rapid Test Kits (Colloidal Gold |
| | Immunochromatography method) |
| Zhongshan Bio-Tech | SARS-CoV-2 IgM/IgG (GICA) |
| Zhuhai Encode Medical Engineering | Novel Coronavirus (COVID-19) IgG/IgM Rapid Test Device |
| Zhuhai Livzon Diagnostics | Diagnostic Kit for IgM/IgG Antibody to Coronavirus (SARS-CoV- |
| | 2) (Colloidal Gold) |

Symptoms to be treated by the present methods, or evaluated during the practice of the present methods may include any of the symptoms of COVID-19, including those discussed below, for example, increased immune response, cytokine storm, ischemia, encephalitis, stroke, loss of smell or taste, respiratory distress, and kidney failure or injury.

Subjects with COVID-19 may suffer from deleterious immune response as a result of the virus. Subjects may have a cytokine storm. In some embodiments, the subject has an elevated cytokine selected from the group consisting of interferons (IFN) such as Type I IFNs (IFN-α and IFN-P), type II IFN (IFN-γ), Lambda IFNs (IFN-λ1, IFN-λ2, and IFN-λ3); interleukins such as IL-1α and IL-1β; chemokines such as CXCL8 (IL-8), CCL2 (monocyte chemoattractant protein 1 [MCP-1]), and CCL11 (eotaxin); colony-stimulating factors (CSFs), such as granulocyte-macrophage colony-stimulating factor (GM-CSF), macrophage colony-stimulating factor (M-CSF), and granulocyte colony-stimulating factor (G-CSF); tumor necrosis factor (TNF); and combinations thereof. The elevated cytokine level may be determined using any medically recognized technique, for example, mass spectrometry, enzyme-linked immunosorbant assay (ELISA), or immunohistochemistry. The elevation may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the subject before infection by coronavirus or relative to a healthy subject. In some embodiments, the method reduces the elevated cytokine by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, of its elevation relative to the subject before infection by coronavirus or relative to a healthy subject or a subject without COVID-19.

In some embodiments, the method further comprises administering one or more immunosuppressant drugs with the aminosterol. The immunosuppressant drug may treat or ameoleorate the immune response to coronavirus and reduce or eliminate the cytokine storm. In some embodiments, the immunosuppressant drug is one or more of tocilizumab; sarilumab; calcineurin inhibitors such as Tacrolimus and Cyclosporine; Antiproliferative agents such as Mycophenolate Mofetil, Mycophenolate Sodium and Azathioprine; mTOR inhibitors such as Sirolimus; and steroids such as Prednisone.

In some embodiments, the subject has reduced blood O₂. The reduction may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the subject before infection by coronavirus or relative to a healthy subject or a subject without COVID-19. In some embodiments, the method increases the reduced blood O₂ by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, of its reduction relative to the subject before infection by coronavirus or relative to a healthy subject. The increase in blood O₂ may be measured using a medically recognized technique, for example, pulse oximeter or blood gas (ABG test).

In some embodiments the subject is suffering from acute respiratory distress syndrome (ARDS). Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. In some embodiments, the subject with COVID-19 is suffering from ischemia. In some embodiments die ischemia comprises ischemia of the kidney or myocardial ischemia.

In some embodiments, the subject is suffering from a blood clot. The blood club may comprise lung thrombi, arterial or white thrombi, venous or red thrombi. In some embodiments, the subject has elevated D-dimer, relative to the subject before infection by coronavirus or relative to a healthy subject or a subject without COVID-19. The elevation may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the subject before infection by coronavirus or relative to a healthy subject. In some embodiments, the method reduces the elevated D-dimer by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, of its elevation relative to the subject before infection by coronavirus or relative to a healthy subject or a subject without COVID-19.

In some embodiments, the subject is in renal failure. Kidney failure is common in COVID-19 subjects and 27% of 85 hospitalized patients in Wuhan, China had kidney failure (Diao et al., 2020). Those with acute kidney injury (AKI), were more than five times as likely to die as COVID-19 patients without it. In some embodiments, the subject has AKI. Diagnosis of kidney failure in COVID-19 subjects can be confirmed by blood tests such as BUN, creatinine, and GFR that measure the buildup of waste products in the blood. Urine tests may be ordered to measure the amount of protein, detect the presence of abnormal cells, or measure the concentration of electrolytes. In some embodiments, the subject is diabetic, thus being at increased risk for kidney injury.

In some embodiments the subject is suffering from encephalitis. Encephalitis is inflammation of the brain. Encephalopathy occurring in COVID-19 patients has been observed (Wadman et al., 2020). Encephalitis symptoms may be assessed in subjects using a medically recognized technique, for example, Magnetic resonance imaging (MRI), Computed tomography scan (also called a CT or CAT scan), blood tests, urine and stool tests, sputum culture, electroencephalogram (EEG), or spinal tap (also called a lumbar puncture). Antiviral medications are often used to treat encephalitis. In one embodiment, the method further comprises administration of one or more antiviral encephalitis drugs. Antiviral encephalitis drugs may include, without limitation acyclovir, ganciclovir, and foscarnet.

Seizures, “sympathetic storm,” loss of sense of smell have also been observed in patients. Sympathetic storm includes an increase in circulating corticoids and catecholamines or a stress response and symptoms can include alterations in level of consciousness, increased posturing, dystonia, hypertension, hyperthermia, tachycardia, tachypnea, diaphoresis, and agitation.

Ocular manifestations of COVID-19 have also been identified as symptoms. These include epiphora, conjunctival congestion, or chemosis (Wu et al., 2020). Viral conjunctivitis is known to present with upper respiratory infections (colds, flus, etc.) and may be a symptom of COVID-19. In some embodiments, the symptom to be treated, prevented, or evaluated is conjunctivitis. Conjunctivitis may be assessed by a medically recognized technique such as slit-lamp, acuity testing, visual analogue scale, McMonnies/Chapman-Davies scale (MC-D), Validated bulbar redness scale (VBR), and Institute for Eye Research scale (IER).

More than half of COVID-19 patients hospitalized in two Chinese centers had elevated levels of enzymes indicating injury to the liver or bile ducts (Zhang et al., 2020). Alanine transaminase (ALT), Aspartate transaminase (AST), Alkaline phosphatase (ALP), and Gamma-glutamyl transpeptidase (GGT) are liver enzymes that may be elevated. The elevation may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the subject before infection by coronavirus or relative to a healthy subject. In some embodiments, the method reduces the elevated liver enzyme by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, of its elevation relative to the subject before infection by coronavirus or relative to a healthy subject or a subject without COVID-19.

Subjects suffering from COVID-19 may require mechanical ventilation. In some embodiments, of the present method, the subject is a mechanically ventilated subject. Administration in this case may comprise pulmonary administration and may be coupled with mechanical ventilation. In one aspect, the composition can be administered to a mechanically ventilated subject. For example, the composition can be administered to the subject using an inhaler or nebulizer device connected to the ventilator via an actuator device. In another aspect, the device can be connected to the ventilator using a spacer chamber. In one embodiment, the composition is administered to the mechanically ventilated subject using a dry powder inhaler, soft mist inhaler, or intratracheal nebulizing catheter adapted for in-line use.

In another aspect, the subject has a comorbidity with COVID-19 selected from the group consisting of diabetes, hypertension, cardiovascular disease, cancer, prior cancer treatment, cerebrovascular disease, chronic obstructive pulmonary disease (COPD), chronic kidney disease, sarcoidosis, obstructive lung disease, idiopathic pulmonary fibrosis (IPF), asthma, chronic bronchitis, emphysema, cystic fibrosis/bronchiectasis, and pneumonia. In another aspect, the subject has a lung disease or respiratory disorder. For all of the methods described herein, the subject can have a lung disease or respiratory disorder. In another embodiment, the coronavirus infection is correlated with pneumonia and/or a lung infection.

The method can additionally comprise administering one or more compounds selected from the group consisting of bronchodilators, inhaled corticosteroids, antibiotics, pulmonary surfactant, mucolytics, biologicals, genes, prostanoids, surfactants, heparin, morphine, furosemide, and combinations thereof. For example, (the bronchodilator can be albuterol, formoterol, arformoterol, fenoterol, metaproterenol, or ipratropium; (b) the inhaled corticosteroid can be budesonide, beclomethasone, or fluticasone; (c) the antibiotic can be tobramycin, amikaci, amikacin, fosfomycin, colistin, ciprofloxacin, ribavirin, and amphotericin B; (d) the surfactant can be Exosurf, Survanta, Curosurf, Infasurf, and KL4; (e) the mucolytic can be N-acetylcysteine or dornase alfa; (f) the biological can be a monoclonal antibody; (g) the gene can be an siRNA; and (h) the prostanoid can be epoprostenol, iloprost, or treprostinil.

In another aspect, the COVID-19 subject has a comorbidity selected from the group consisting of diabetes, hypertension, cardiovascular disease, cancer, prior cancer treatment, cerebrovascular disease, chronic obstructive pulmonary disease (COPD), chronic kidney disease, sarcoidosis, obstructive lung disease, idiopathic pulmonary fibrosis (IPF), asthma, chronic Bronchitis, emphysema, cystic fibrosis/bronchiectasis, and pneumonia. In another aspect, the subject has a lung disease or respiratory disorder.

In an exemplary aspect, the disclosure encompasses a method of treating a subject in need, wherein the subject has been diagnosed with a coronavirus infection, is suspected of having a coronavirus infection, and/or is at risk of developing a coronavirus infection, wherein the coronavirus is SARS-CoV-2, wherein the method comprises administering to the subject a therapeutically effective amount of a composition comprising at least one aminosterol via inhalation, pulmonary, and/or nasal administration, wherein the aminosterol is in a lactate or dilactate salt form. In another expect, the disclosure encompasses a method of inhibiting viral replication of SARS-CoV-2 in a subject, wherein the method comprises administering to the subject a therapeutically effective amount of a composition comprising at least one aminosterol via inhalation, pulmonary and/or nasal administration, wherein the aminosterol is in a lactate or dilactate salt form.

In such methods, following administration, viral load in the subject can be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the subject before administration of aminosterol, wherein the reduction is measured by a medically recognized technique selected from the group consisting of tunable resistive pulse sensing (TRPS), flow cytometry, quantitative polymerase chain reaction (qPCR), enzyme-linked immunosorbent assay (ELISA), and transmission electron microscopy (TEM).

In addition, inhalation, pulmonary and/or nasal administration can be via a device or method selected from the group consisting of a nebulizer, pressurized nebulizer, a jet nebulizer, constant-output jet nebulizer, inspiratory synchronized jet nebulizer, ultrasonic nebulizer, constant-output ultrasonic nebulizer, vibrating-mesh nebulizer, constant-output vibrating-mesh nebulizer, vibrating mesh nebulizer, soft mist inhaler, metered dose inhaler (MDI), pressurized metered-dose inhaler, Dry Powder Inhaler, and Intratracheal Nebulizing Catheter. In one aspect, the method uses a single dose Dry Powder Inhaler (DPI) device for administration. In another aspect, the DPI device delivers the composition in the form of a dry powder contained in a capsule. Further, the device can be a breath actuated inhaler.

The aminosterol composition can be in a dry powder dosage form. Alternatively, the aminosterol composition can be in a liquid dispersion dosage form.

In one aspect, the aminosterol composition can be administered to a mechanically ventilated subject. For example, the composition can be administered to the subject using an inhaler or nebulizer device connected to the ventilator via an actuator device. In another aspect, the device can be connected to the ventilator using a spacer chamber. In yet a further aspect, the ventilation comprises delivery of a tidal volume selected from the group consisting of about 100 mL to about 200 ml, about 200 ml to about 300 ml, about 300 ml to about 400 ml, about 400 ml to about 500 ml, about 500 ml to about 600 mL, about 600 ml to about 700 mL, or greater than about 700 ml. In another aspect, the ventilation comprises inspiratory flow of about 10 L/min to about 20 L/min, about 20 L/min to about 30 L/min, about 30 L/min to about 40 L/min, about 40 L/min to about 50 L/min, about 50 L/min to about 60 L/min, or greater than about 60 L/min.

In one aspect, the composition comprising at least one aminosterol can be heated before administering.

In the methods described herein, the composition can be administered to the mechanically ventilated subject using a dry powder inhaler, soft mist inhaler, or intratracheal nebulizing catheter adapted for in-line use.

In another aspect, the coronavirus infection can be correlated with pneumonia and/or a lung infection.

In one aspect, the method additionally comprises administering one or more compounds selected from the group consisting of bronchodilators, inhaled corticosteroids, antibiotics, pulmonary surfactant, mucolytics, biologicals, genes, prostanoids, surfactants, heparin, morphine, furosemide, and combinations thereof. For example, the bronchodilator can be one or more selected from albuterol, formoterol, arformoterol, fenoterol, metaproterenol, and ipratropium. In addition, the inhaled corticosteroid can be one or more selected from budesonide, beclomethasone, and fluticasone. Further, the antibiotic can be one or more selected from tobramycin, amikaci, amikacin, fosfomycin, colistin, ciprofloxacin, ribavirin, and amphotericin B. In addition, the surfactant can be one or more selected from Exosurf, Survanta, Curosurf, Infasurf, and KL4. In one aspect, the mucolytic can be one or more selected from N-acetylcysteine and dornase alfa. The biological can be a monoclonal antibody, and the gene can be an siRNA. The prostanoid can be one or more selected from epoprostenol, iloprost, and treprostinil.

In one aspect, the subject has a comorbidity selected from one or more of diabetes, hypertension, cardiovascular disease, cancer, prior cancer treatment, cerebrovascular disease, chronic obstructive pulmonary disease (COPD), chronic kidney disease, sarcoidosis, obstructive lung disease, idiopathic pulmonary fibrosis (IPF), asthma, chronic Bronchitis, emphysema, cystic fibrosis/bronchiectasis, and pneumonia. Further, the subject can have a lung disease or respiratory disorder.

### VI. Patient Populations

The disclosed aminosterols and compositions comprising the same can be used to treat a range of subjects, including human and non-human animals, including mammals, as well as immature and mature animals, including human children and adults. The human subject to be treated can be an infant, toddler, school-aged child, teenagers, young adult, adult, or elderly patient.

In embodiments disclosed herein relating to prevention, particular patient populations may be selected based on being “at risk for” the development of any of the conditions disclosed herein. For example, genetic markers of the condition or family history may be used as signs to identify subjects likely to develop the particular condition. Thus, in some embodiments, prevention may involve first identifying a patient population at risk of developing the condition. Alternatively, certain symptoms are considered early signs of particular disorders. Thus, in some embodiments, a patient population may be selected for being “at risk” for developing the condition based on age and experiencing symptoms associated with the condition. Further genetic or hereditary signs may be used to refine the patient population.

### VII. Kits

Aminosterol formulations or compositions of the disclosure may be packaged together with or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The disclosure also provides a pharmaceutical pack or kit comprising one or more containers filled with one or more aminosterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat-sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

### VIII. Combination Therapy

In the methods of the disclosure, the aminosterol compositions may be administered alone or in combination with one or more other therapeutic agents. An example of an additional therapeutic agent is one known to treat the condition the aminosterol is being administered to treat.

In some embodiments, the aminosterol compositions may be administered alone or in combination with one or more other therapeutic agents, such as an antimicrobial agent. The additional therapeutic or antimicrobial agent can be administered via any pharmaceutically acceptable method, including but not limited to pulmonary or inhaled administration. In addition, an example of an additional therapeutic agent is one known to treat the condition the aminosterol is being administered to treat.

In some embodiments, the other therapeutic agent is an antimicrobial agent selected from the group comprising, but not limited to, antibiotics, antibodies, antibacterial enzymes, peptides, and lanthione-containing molecules. In some embodiments, the antimicrobial interferes with or inhibits cell wall synthesis. In some embodiments, the antimicrobial is selected from the group including, but not limited to, β-lactams, cephalosporins, glycopeptides, aminoglycosides, sulfonomides, macrolides, folates, polypeptides and combinations thereof. In some embodiments, the antimicrobial interferes with protein synthesis (e.g., glycosides, tetracyclines and streptogramins).

In some embodiments, co-administration with a composition comprising aminosterol permits administering a lower dose of an antimicrobial agent than would be administered without such co-administration.

Examples of drugs that are typically administered via aerosol to mechanically ventilated subjects are shown in Table 1B below.

TABLE 1B
Therapeutic Aerosols in Mechanically [symbol] *

| Drugs | Delivery Device for Administration |
|---|---|
| [symbol] | pMDI with spacer/ [symbol] nebulizer/ [symbol] nebulizer |
| Corticosteroids: | |
| [symbol] | [symbol] nebulizer |
| [symbol] | pMDI with spacer |
| [symbol] | pMDI with spacer |
| Antibiotics: | |
| [symbol] | [symbol] nebulizer/ [symbol] nebulizer |
| [symbol] | Small partical aerosol [symbol] |
| [symbol] | [symbol] nebulizer |
| [symbol] | [symbol] nebulizer/ [symbol] nebulizer |
| [symbol] | [symbol] nebulizer |
| [symbol] | [symbol] nebulizer/ [symbol] nebulizer |
| [symbol] | [symbol] nebulizer/ [symbol] nebulizer |
| [symbol] | [symbol] nebulizer |
| [symbol] | [symbol] nebulizer |

*

[symbol]

[symbol]

[symbol]

[symbol]

[symbol]

indicates data missing or illegible when filed

In methods of treating, preventing, and/or slowing the onset or progression of AD or a related symptom, the aminosterol composition can be co-administered or combined with drugs commonly prescribed to treat PD or related symptoms, such as levodopa (usually combined with a dopa decarboxylase inhibitor or COMT inhibitor), dopamine agonists and MAO-B inhibitors. Exemplary dopa decarboxylase inhibitors are carbidopa and benserazide. Exemplary COMT inhibitors are tolcapone and entacapone. Dopamine agonists include, for example, bromocriptine, pergolide, pramipexole, ropinirole, piribedil, cabergoline, apomorphine, lisuride, and rotigotine. MAO-B inhibitors include, for example, selegiline and rasagiline. Other drugs commonly used to treat PD include, for example, amantadine, anti cholinergi cs, clozapine for psychosis, cholinesterase inhibitors for dementia, and modafinil for daytime sleepiness.

In methods of treating, preventing, and/or slowing the onset or progression of AD or related symptoms associated with AD, the aminosterol composition can be co-administered or combined with drugs commonly prescribed to treat AD or related symptoms, such as glutamate, antipsychotic drugs, huperzine A, acetylcholinesterase inhibitors and NMDA receptor antagonists such as memantine (Akatinol®, Axura®, Ebixa®/Abixa®, Memox® and Namenda®). Examples of acetylcholinesterase inhibitors are donepezil (Aricept®), galantamine (Razadyne®), and rivastigmine (Exelon®).

In methods of treating, preventing, and/or slowing the onset or progression of diabetes or related symptoms associated with diabetes and/or diabetes mellitus, including both Type 1 and Type 2 diabetes, or neuropathy of diabetes, the aminosterol composition can be co-administered or combined with drugs commonly prescribed to treat diabetes mellitus or related symptoms, such as insulin (NPH insulin or synthetic insulin analogs) (e.g., Humulin*, Novolin®) and oral antihyperglycemic drugs. Oral antihyperglycemic drugs include but are not limited to (1) biguanides such as metformin (Glucophage®); (2) Sulfonylureas such as acetohexamide, chlorpropamide (Diabinese®), glimepiride (Amaryl®), glipizide (Glucotrol®), tolazamide, Tolbutamide, and glyburide (Diabeta®, Micronase®); (3) Meglitinides such as repaglinide (Prandin®) and nateglinide (Starlix®); (4) Thiazolidinediones such as rosiglitazone (Avandia®) and pioglitazone (Actos®); (5) Alpha-glucosidase inhibitors such as acarbose (Precose®) and miglitol (Glyset®); (6) Dipeptidyl peptidase-4 inhibitors such as Sitagliptin (Januvia®); (7) Glucagon-like peptide agonists such as exenatide (Byetta®); and (8) Amylin analogs such as pramlintide (Symlin®).

In methods of treating, preventing, and/or slowing the onset or progression of HD or related symptoms associated with Huntington's chorea or disease, the aminosterol composition can be co-administered or combined with drugs commonly prescribed to treat Huntington's chorea or related symptoms, such as medications prescribed to help control emotional and movement problems associated with Huntington's chorea. Such medications include, but are not limited to, (1) antipsychotic drugs, such as haloperidol and clonazepam; (2) drugs used to treat dystonia, such as acetylcholine regulating drugs (trihexyphenidyl, benztropine (Cogentin®), and procyclidine HCl); GABA-regulating drugs (diazepam (Valium®), lorazepam (Ativan®), clonazepam (Klonopin®), and baclofen (Lioresal®)); dopamine-regulators (levodopa/carbidopa (Sinemet®), bromocriptine (parlodel), reserpine, tetrabenazine); anticonvulsants (carbamazepine (Tegretol®) and botulinum toxin (Botox®)); and (3) drugs used to treat depression (fluoxetine, sertraline, and nortriptyline). Other drugs commonly used to treat HD include amantadine, tetrabenazine, dopamine blockers, and co-enzyme Q₁₀.

In methods of treating, preventing, and/or slowing the onset or progression of peripheral sensory neuropathy or related symptoms associated with peripheral sensory neuropathy, the aminosterol composition can be co-administered or combined with drugs commonly prescribed to treat peripheral sensory neuropathy or related symptoms. Peripheral sensory neuropathy refers to damage to nerves of the peripheral nervous system, which may be caused either by diseases of or trauma to the nerve or the side-effects of systemic illness. Drugs commonly used to treat this condition include, but are not limited to, neurotrophin-3, tricyclic antidepressants (e.g., amitriptyline), antiepileptic therapies (e.g., gabapentin or sodium valproate), synthetic cannabinoids (Nabilone) and inhaled cannabis, opiate derivatives, and pregabalin (Lyrica®).

In methods of treating, preventing, and/or slowing the onset or progression of traumatic head and/or spine injury or related symptoms associated with traumatic head and/or spine injury, the aminosterol composition can be co-administered or combined with drugs commonly prescribed to treat traumatic head and/or spine injury or related symptoms, such as analgesics (acetaminophen, NSAIDs, salicylates, and opioid drugs such as morphine and opium) and paralytics.

In methods of treating, preventing, and/or slowing the onset or progression of stroke or related symptoms associated with stroke, the aminosterol composition can be co-administered or combined with drugs commonly prescribed to treat stroke or related symptoms, such as aspirin, clopidogrel, dipyridamole, tissue plasminogen activator (tPA), and anticoagulants (e.g., alteplase, warfarin, dabigatran).

In methods of treating, preventing, and/or slowing the onset or progression of ALS or related symptoms associated with ALS, the aminosterol composition can be co-administered or combined with drugs commonly prescribed to treat Amyotrophic lateral sclerosis or related symptoms, such as riluzole (Rilutek®), KNS-760704 (an enantiomer of pramipexole), olesoxime (TRO19622), talampanel, arimoclomol, medications to help reduce fatigue, ease muscle cramps, control spasticity, reduce excess saliva and phlegm, control pain, depression, sleep disturbances, dysphagia, and constipation.

In methods of treating, preventing, and/or slowing the onset or progression of MS or related symptoms associated with multiple sclerosis, the aminosterol composition can be co-administered or combined with drugs commonly prescribed to treat multiple sclerosis or related symptoms, such as corticosteroids (e.g., methylprednisolone), plasmapheresis, fingolimod (Gilenya®), interferon beta-la (Avonex®, CinnoVex®, ReciGen® and Rebif*), interferon beta-lb (Betaseron® and Betaferon®), glatiramer acetate (Copaxone®), mitoxantrone, natalizumab (Tysabri®), alemtuzumab (Campath®), daclizumab (Zenapax®), rituximab, dirucotide, BHT-3009, cladribine, dimethyl fumarate, estriol, fingolimod, laquinimod, minocycline, statins, temsirolimus teriflunomide, naltrexone, and vitamin D analogs.

In methods of treating, preventing, and/or slowing the onset or progression of cerebral palsy or related symptoms associated with cerebral palsy, the aminosterol composition can be co-administered or combined with drugs commonly prescribed to treat cerebral palsy or related symptoms, such as botulinum toxin A injections.

In methods of treating, preventing, and/or slowing the onset or progression of epilepsy or related symptoms associated with epilepsy, the aminosterol composition can be co-administered or combined with drugs commonly prescribed to treat epilepsy or related symptoms, such as anticonvulsants (e.g., carbamazepine (Tegretol®), clorazepate (Tranxene®), clonazepam (Klonopin®), ethosuximide (Zarontin®), felbamate (Felbatol®), fosphenytoin (Cerebyx®), gabapentin (Neurontin®), lacosamide (Vimpat®), lamotrigine (Lamictal®), levetiracetam (Keppra®), oxcarbazepine (Trileptal®), phenobarbital (Luminal®), phenytoin (Dilantin®), pregabalin (Lyrica®), primidone (Mysoline®), tiagabine (Gabitril®), topiramate (Topamax®), valproate semisodium (Depakote®), valproic acid (Depakene®), and zonisamide (Zonegran®), clobazam (Frisium®), vigabatrin (Sabril®), retigabine, brivaracetam, seletracetam, diazepam (Valium® and Diastat*), lorazepam (Ativan®), paraldehyde (Paral®), midazolam (Versed®), pentobarbital (Nembutal®), acetazolamide (Diamox®), progesterone, adrenocorticotropic hormone (ACTH and Acthar®), various corticotropic steroid hormones (prednisone), and bromide.

In methods of treating, preventing, and/or slowing the onset or progression of cognitive impairment or related symptoms associated with cognitive impairment, the aminosterol composition can be co-administered or combined with drugs commonly prescribed to treat cognitive impairment, such as donepezil (Aricept®), galantamine (Razadyne®), rivastigmine (Exelon®); and stimulants such as caffeine, amphetamine (Adderall®), lisdexamfetamine (Vyvanse®), and methylphenidate (Ritalin®); NMDA antagonists such as memantine (Nameda®); supplements such as ginko biloba, L-theanine, piracetam, oxiracitam, aniracetam, tolcapone, atomoxetine, ginseng, and salvia officinalis.

In the methods of treating, preventing, and/or slowing the onset or progression of malignancy or related symptoms associated with malignancies, the aminosterol composition can be co-administered or combined with drugs commonly used to treat malignancies. These include all known cancer drugs, such as but not limited to those listed at http://www.cancer.gov/cancertopics/druginfo/alphalist as of May 5, 2014, which is specifically incorporated by reference. In one embodiment, the drug commonly used to treat malignancies may be selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, 5FC, accutane hoffmann-la roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIB021, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and any combination thereof.

In the methods of treating, preventing, and/or slowing the onset or progression of depression or related symptoms associated with depression, the aminosterol composition can be co-administered or combined with drugs commonly used to treat depression. These include selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressin®, Vagran®), quinupramine (Kinupril®, Kevopril®), tiazesim (Altinil®), tofenacin (Elamol®, Tofacine®), amineptine (Survector®, Maneon®), tianeptine (Stablon®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); and N-methyl D-aspartate (NMDA) antagonists such ketamine (Ketalar®).

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents administered first, followed by the second. The regimen selected can be administered concurrently since activation of the aminosterol induced response does not require the systemic absorption of the aminosterol into the bloodstream and thus eliminates concern over the likelihood systemic of drug-drug interactions between the aminosterol and the administered drug.

### IX. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

The terms “pharmacologically effective amount” or “therapeutically effective amount” of a composition, aminosterol or agent, as provided herein, refer to a nontoxic but sufficient amount of the composition, aminosterol or agent to provide the desired response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., mass, temperature, time, and concentration, including ranges, are approximations which are varied (+) or (−) by increments of 1, 5, or 10%. It is to be understood, although not always explicitly stated that all numerical designations are preceded by the term “about.”

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

“Optional” or “optionally” means that the subsequently described circumstance may or may not occur, so that the description includes instances where the circumstance occurs and instances where it does not.

“Pharmaceutically acceptable excipient or carrier” refers to an excipient that may optionally be included in the compositions of the disclosure and that causes no significant adverse toxicological effects to the patient.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used in the description of the disclosure and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “aminosterol” refers to an amino derivative of a sterol. A non-limiting example of a suitable aminosterol for use in the composition and methods disclosed herein included Compound VI (ENT-06).

The term “administering” as used herein includes prescribing for administration as well as actually administering and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

A “subject in need” is a human or animal at risk of a microbial infection, or which has contracted a microbial infection. Preferably, the lactate or dilactate aminosterol salt is a pharmaceutical grade. The composition can further comprise one or more pharmaceutically acceptable excipients. Further, the aminosterol is present in an amount sufficient to produce an antimicrobial effect. As used herein, the terms “at risk for disease” and “at risk for infection” refer to a subject that is predisposed to experiencing a particular disease and/or infection. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk (e.g., a subject may be “at risk for disease” simply by being exposed to and interacting with other people that carry a risk of transmitting a pathogen), nor is it intended that the present invention be limited to any particular disease and/or infection.

As used herein, the term “microorganism” or “microbe” (including descriptors such as “microbial”) refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

“Prodrug” a prodrug is a medication or compound that, after administration, is metabolized (i.e., converted within the body) into a pharmacologically active drug, for example, Compound VI. In some embodiments, a prodrug comprises a derivative of Compound VI, wherein the alcohol and/or the carboxylate has been esterified.

“Optionally substituted” refers to a group selected from that group and a substituted form of that group. A “substituted” group, refers to that group substituted with a chemical substituent, for example be replacement of a C—H bond with a bond between that C and the substituent. In one embodiment, substituents are selected from, for example, CF₃, OCF₃, halo, haloaryl, C₁-C₆ alkoxy, acyl, propionyl, butyrl, acylamino, acyloxy, amino, substituted amino, aminocarbonyl, aminothiocarbonyl, aminocarbonylamino, aminothiocarbonylamino, carboxyl ester, carboxyl ester amino, (carboxyl ester)oxy, haloalkyl, aryloxy, haloalkoxy, hydroxyl, thiol, dihydroxy, aminohydroxy, carboxy, amido, sulfoxy, sulfonyl, haloaryloxy, aryl, benzyl, benzyloxy, heteroaryl, nitrile, C₁-C₆ alkyl, C₁-C₆ alkenyl, C₁-C₆ alkynyl, C₃-C₆ cycloalkyl, C₁-C₆ haloalkyl, C₁-C₆ haloalkenyl, C₁-C₆ haloalkynyl, C₃-C₆ halocycloalkyl, C₆-C₁₀ aryl, C₃-C₈ cycloalkyl, C₂-C₁o heterocyclyl, C₁-C₁₀ heteroaryl, —N₃, nitro, —CO₂H or a C₁-C₆ alkyl ester thereof, or combinations thereof.

### X. Examples

### Example 1: Rejuvenation of RNA Transcriptome in the Gut

Aging involves a depletion of gene expression in the gut. Comparison of the images showing mucosal tissue in the stomach of a young mouse (20 week, FIG. 1A) versus an old mouse (78 week, FIG. 1B) shows a reduced thickness of the mucosal layer in the older specimen. This reduction in mucosa is associated with a reduced RNA transcriptome in the stomach in aged mice vs. young mice, see Table 2 below.

The dosing schedule used to determine the effect of orally administered squalamine and ENT-02 (MSI-1436) on the GI tracts of young and old mice was as follows. Male C57B1/6 mice, aged 20 and 78 weeks, were obtained from Jackson labs. Animals were exposed to 12 hr light dark cycles and provided Teklad standard mouse diet and water ad lib. Animals were assigned to the treatment groups shown in Table 1C.

TABLE 1C

| | | | | | Number |
|---|---|---|---|---|---|
| | Dose | | Dose | Age on | of |
| | Level* | Concentration | Volume | Day 1 | Animals |
| Group | (mg/kg/day) | (mg/ml) | (ml/kg) | (weeks) | Male |
| 1. Control (vehicle) | 0 | 0 | 10.0 | 20 | 5 |
| 2. Squalamine | 40 | 4 | 10.0 | 20 | 5 |
| 3. ENT-02 (MSI-1436) | 40 | 4 | 10.0 | 20 | 5 |
| 4. Control (vehicle) | 0 | 0 | 10.0 | 78 | 5 |
| 5. Squalamine | 40 | 4 | 10.0 | 78 | 5 |
| 6. ENT-02 (MSI-1436) | 40 | 4 | 10.0 | 78 | 5 |

Animals were dosed once daily by oral gavage in the morning for a total of 14 days. Animals were fasted 3-4 hours prior to dosing and 1 hour following. The test article was dissolved in 0.5% hydroxypropylcellulose in water. On day 15 the animals were euthanized by C₀₂ asphyxiation, necropsied, and tissues prepared for histology and RNAseq analysis.

The GI tracts of the animals were sectioned into stomach, duodenum, jejunum, ileum, caecum, colon, and rectum. The tissues were then sent for histology, and the transcriptomes analyzed by RNAseq. Table 2 shows the respective mRNA amounts in young and old mouse stomach.

As shown in Table 3 below, mRNA levels for all of the genes in the table showed a significant increase after treatment with squalamine. This suggests that squalamine, and by extension structurally related aminosterols, such as Compound VI and derivatives thereof, have a rejuvenating effect in the gut.

TABLE 2
Respective mRNA amounts in young and old mouse stomach
Epithelial Barrier Functions

| Gene | Young | Old | P Value |
|---|---|---|---|
| caspase_14 | 9.228 | 0.835 | 8.45E−10 |
| collagen_type_XVII_alpha_1 | 9.835 | 2.804 | 2.07E−04 |
| corneodesmosin | 12.75 | 4.295 | 6.74E−04 |
| cornifelin | 23.74 | 6.442 | 3.01E−05 |
| cystatin_E/M | 9.41 | 1.551 | 5.68E−07 |
| dermokine | 117.5 | 23.86 | 1.34E−07 |
| desmocollin_1 | 16.09 | 4.175 | 2.6E−05 |
| desmoglein_1_beta | 9.592 | 2.625 | 1.41E−04 |
| Filaggrin | 21.67 | 2.207 | 3.81E−11 |
| gap_junction_protein_beta_4 | 1.882 | 0.239 | 2.52E−04 |
| gap_junction_protein_beta_6 | 2.125 | 0.298 | 2.11E−04 |
| H19_imprinted_maternally_expressed_transcript | 39.95 | 1.909 | 1.59E−17 |
| hornerin | 13.6 | 3.519 | 3.23E−05 |
| kallikrein_related- | 10.14 | 0.656 | 1.33E−11 |
| peptidase_7_(chymotryptic_stratum_{—} | | | |
| keratin_1 | 332.6 | 69.79 | 1.72E−07 |
| keratin_10 | 337.7 | 45.93 | 8.03E−11 |
| keratinocyte_differentiation_associated_protein | 184.6 | 37.58 | 1.21E−07 |
| keratinocyte_expressed_proline-rich | 12.93 | 2.267 | 3.87E−07 |
| late_cornified_envelope_1A1 | 17.85 | 2.625 | 1.42E−08 |
| late_cornified_envelope_1A2 | 23.43 | 4.056 | 6.01E−08 |
| late_cornified_envelope_1B | 9.774 | 1.491 | 2.05E−07 |
| late_cornified_envelope_1C | 10.93 | 1.73 | 1.90E−07 |
| late_cornified_envelope_1E | 5.767 | 1.074 | 1.23E−05 |
| late_cornified_envelope_1F | 8.742 | 1.551 | 1.67E−06 |
| late_cornified_envelope_1G | 8.378 | 1.611 | 4.59E−06 |
| late_cornified_envelope_1H | 5.282 | 1.312 | 2.14E−04 |
| late_cornified_envelope_1I | 8.682 | 1.074 | 3.37E−08 |
| late_cornified_envelope_1J | 5.16 | 0.895 | 1.01E−05 |
| late_cornified_envelope_1L | 4.553 | 0.596 | 1.82E−06 |
| late_cornified_envelope_1M | 16.76 | 1.133 | 5.22E−13 |
| late_cornified_envelope_3C | 5.525 | 1.491 | 3.92E−04 |
| late_cornified_envelope_3E | 5.889 | 1.491 | 3.92E−04 |
| late_cornified_envelope_3F | 10.81 | 3.281 | 2.66E−04 |
| lectin_galactose_binding_soluble_7 | 139.3 | 25.77 | 2.70E−08 |
| loricrin | 275.3 | 45.75 | 3.37E−09 |
| sciellin | 11.11 | 3.221 | 1.56E−04 |

Muscle Tissue

| | | | |
|---|---|---|---|
| myoglobin | 47.9 | 2.923 | 4.10E−16 |
| myosin_binding_protein_C_slow-type | 15.06 | 1.133 | 4.01E−12 |
| myosin_heavy_polypeptide_1_skeletal_muscle | 73.46 | 2.982 | 3.47E−20 |
| myosin_heavy_polypeptide_8_skeletal_muscle | 90.46 | 3.34 | 2.67E−21 |
| myosin_light_chain_phosphorylatable_fast_ske | 30.23 | 4.235 | 1.42E−09 |
| myosin_light_polypeptide_3 | 26.77 | 1.312 | 2.72E−16 |
| myozenin_1 | 6.617 | 0.418 | 3.87E−10 |
| myozenin_2 | 4.31 | 0.239 | 7.95E−09 |
| titin-cap | 17.36 | 0.418 | 3.66E−18 |

TABLE 3
respective mRNA amounts in old mice versus old mice treated with squalamine (ENT-01)
Epithelial Barrier Functions

| Gene | Old | Old + Ent-01 | P Value |
|---|---|---|---|
| caspase_14 | 0.795 | 6.077 | 2.25E−07 |
| collagen_type_XVII_alpha_1 | 2.668 | 9.509 | 1.43E−04 |
| corneodesmosin | 4.087 | 7.933 | 4.10E−02 |
| cornifelin | 6.131 | 22.17 | 4.07E−05 |
| cystatin_E/M | 1.476 | 8.721 | 6.89E−07 |
| dermokine | 22.71 | 88.45 | 5.65E−06 |
| desmocollin_1 | 3.974 | 8.102 | 2.83E−02 |
| desmoglein_1_beta | 2.498 | 7.089 | 2.14E−03 |
| filaggrin | 2.1 | 16.32 | 2.05E−09 |
| gap_junction_protein_beta_4 | 0.227 | 1.294 | 3.15E−03 |
| gap_junction_protein_beta_6 | 0.284 | 1.575 | 1.59E−03 |
| H19_imprinted_maternally_expressed_transcript | 1.817 | 13.73 | 5.79E−09 |
| hornerin | 3.349 | 11.82 | 1.09E−04 |
| kallikrein_related- | 0.624 | 5.908 | 3.74E−08 |
| peptidase_7_(chymotryptic_stratum_{—} | | | |
| keratin_1 | 66.42 | 348.9 | 3.28E−08 |
| keratin_10 | 43.71 | 310.3 | 1.56E−10 |
| keratinocyte_differentiation_associated_protein | 35.77 | 181.2 | 7.27E−08 |
| keratinocyte_expressed_proline-rich | 2.157 | 7.99 | 1.36E−04 |
| late_cornified_envelope_1A1 | 2.498 | 10.97 | 1.03E−05 |
| late_cornified_envelope_1A2 | 3.86 | 17.78 | 2.08E−06 |
| late_cornified_envelope_1B | 1.419 | 8.102 | 1.33E−06 |
| late_cornified_envelope_1C | 1.646 | 7.202 | 3.26E−05 |
| late_cornified_envelope_1E | 1.022 | 4.501 | 1.17E−04 |
| late_cornified_envelope_1F | 1.476 | 6.47 | 3.92E−05 |
| late_cornified_envelope_1G | 1.533 | 4.557 | 2.78E−03 |
| late_cornified_envelope_1H | 1.249 | 4.164 | 1.38E−03 |
| late_cornified_envelope_1I | 1.022 | 7.708 | 8.29E−08 |
| late_cornified_envelope_1J | 0.852 | 3.376 | 6.62E−04 |
| late_cornified_envelope_1L | 0.568 | 2.644 | 5.13E−04 |
| late_cornified_envelope_1M | 1.079 | 8.158 | 6.04E−08 |
| late_cornified_envelope_3C | 1.419 | 3.263 | 2.79E−02 |
| late_cornified_envelope_3E | 1.419 | 4.332 | 2.50E−03 |
| late_cornified_envelope_3F | 3.122 | 9.115 | 1.15E−03 |
| lectin_galactose_binding_soluble_7 | 24.52 | 142.5 | 7.67E−09 |
| loricrin | 43.54 | 209.5 | 1.63E−07 |
| sciellin | 3.066 | 9.79 | 4.25E−04 |

Muscle Tissue

| | | | |
|---|---|---|---|
| myoglobin | 2.782 | 17.72 | 2.49E−08 |
| myosin_binding_protein_C_slow-type | 1.079 | 3.173 | 1.42E−03 |
| myosin_heavy_polypeptide_1_skeletal_muscle | 2.838 | 11.03 | 4.18E−05 |
| myosin_heavy_polypeptide_8_skeletal_muscle | 3.179 | 14.97 | 2.20E−06 |
| myosin_light_chain_phosphorylatable_fast_ske | 4.031 | 15.92 | 1.79E−05 |
| myosin_light_polypeptide_3 | 1.249 | 10.58 | 4.49E−09 |
| myozenin_1 | 0.397 | 2.476 | 1.19E−04 |
| myozenin_2 | 0.227 | 1.519 | 8.72E−04 |
| titin-cap | 0.397 | 3.376 | 3.15E−06 |

### Example 2: Synthetic Methods for the Preparation of ENT-06

This example describes synthetic methods of making compounds described herein.

Preparation of Compound 2:

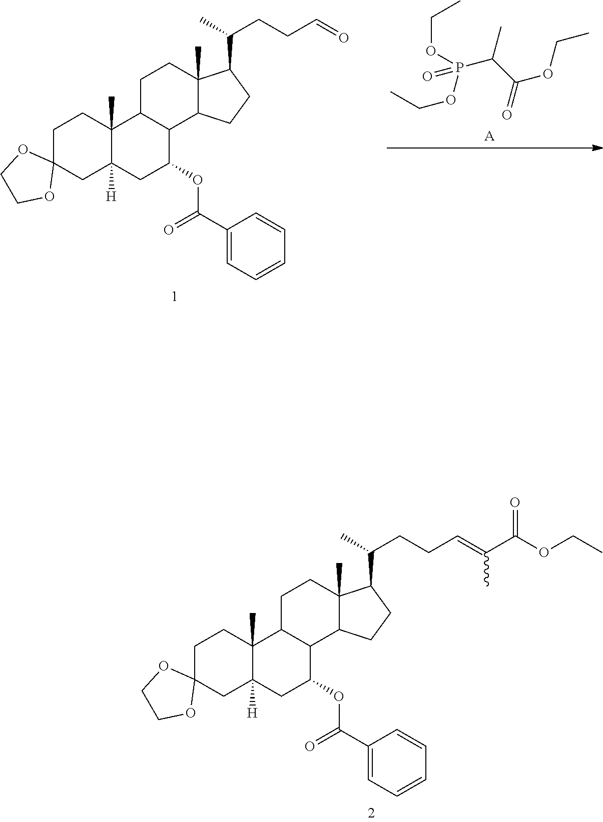

The phosphonate (A, 3.69 g, 15 mmol) was added to anhydrous tetrahydrofuran (100 mL) and chilled in a salt ice bath to −0° C. Potassium tert-butoxide (1.72 g, 15 mmol) was added with vigorous magnetic stirring under nitrogen and the reaction was allowed to stir for 30 min. Compound 1 (8.0 g, 15 mmol) was added and dissolved in tetrahydrofuran (80 mL), the ice bath was removed and the reaction was allowed to warm to RT overnight. The reaction after approximately 16 h total was worked up by partitioning between hexane/ethyl acetate (50/50, 400 mL) and water (400 mL). The organic layer was washed with an additional portion of water (100 mL) and the organic layer was dried over Na₂SO₄, filtered, and the solvent removed in vacuo. The residue was redissolved in a minimal amount of hexane/ethyl acetate 3/1 and passed through a plug of silica gel-3×9 in.

The eluant was then roto-evaporated to yield Compound 2 (8.3 g, 12.4 mmol, 83%) of satisfactory purity to utilize in the next step without further purification,

¹H NMR (CDCl3, 300 MHz) δ 8.10-8.07 (m, 2H), 7.60-7.57 (m, 1H), 7.52-7.47 (m, 2H), 6.7, 5.9 (t, 1H), 5.17 (m, 1H), 4.21-4.12 (m, 2H), 3.92-3.88 (m, 4H), 2.06 (s, 3H), 2.2-1.0 (m, 29H), 0.95 (d, 3H, J=7 Hz), 0.90 (s, 3H), 0.69 (s, 3H); MS (ES+) 485.45 (M-C₇H₇O₂+H).

Preparation of Compound 3:

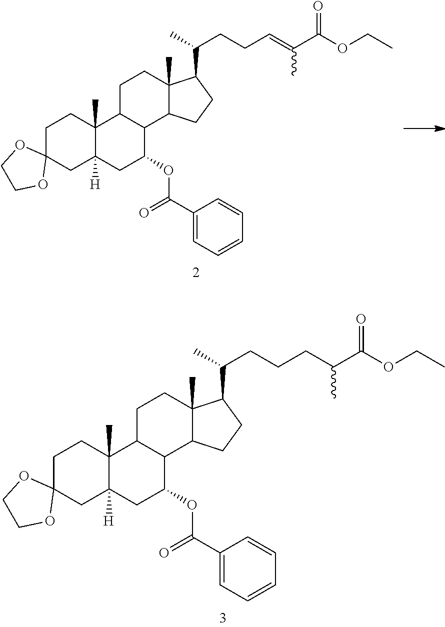

Compound 2 (8.25 g, 13.5 mmol) was dissolved in anhydrous ethanol and 10% Pd on C (400 mg) was added under N₂ in a Parr bottle (500 mL). The flask was flushed and filled 2× with vacuum and N₂ then hydrogenated at 50 psi for 24 h. The uptake of hydrogen had slowed to a near stop, but TLC showed a possible trace of starting material. An additional portion of catalyst (400 mg) was added, and the reaction was allowed an additional 12 h.

Filtration of catalyst and removal of the solvent in vacuo gave the saturated product in quantitative yield Compound 3 (8.25 g, 13.5 mmol),

¹H NMR (CDCl3, 300 MHz) δ 8.09-8.06 (m, 2H), 7.58-7.56 (m, 1H), 7.55-7.45 (m, 2H), 5.16 (m, 1H), 4.12-4.05 (m, 2H), 3.90-3.85 (m, 4H), 2.39-2.36 (m, 1H), 2.0-1.0 (m, 35H), 1.11 (d, 3H, J=7 Hz), 0.88 (s, 3H), 0.67 (s, 3H); MS (ES+) 487.46 (M-C₇H₇O₂+H).

Preparation of Compound 4:

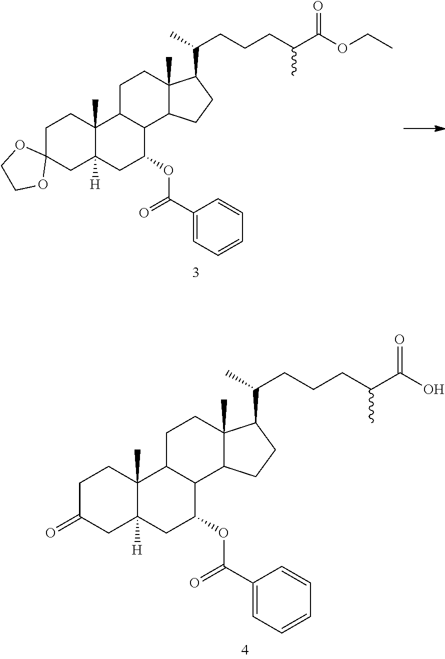

Compound 3 (8.2 g, 13.5 mmol) was dissolved in 3/1/1 tetrahydrofuran/methanol/1M KOH (˜100 mL) and stirred until hydrolysis of the ethyl ester appeared to be complete by TLC. There was no evidence of benzoate hydrolysis under these conditions. The solution was neutralized with 1 M hydrochloric acid solution, evaporated to remove the organic solvent, treated with acetone (˜100 mL), and evaporated again to ensure removal of any methanol. Acetone (˜250 mL) was added to the flask and 3M HCl was added to lower the pH to the point where it registered in the 1-2 range by pH paper. The hydrolysis of the ketal was carried out overnight at RT, water was then added to the flask, and the majority of the acetone was removed in vacuo. The material was partitioned between ethyl acetate and water, and then the organic layer washed with brine.

The organic layer was dried in vacuo to give Compound 4 (6.36 g, 11.9 mmol, 87%) of satisfactory purity to be utilized without further purification,

¹H NMR (CDCl3, 300 MHz) δ 8.05-8.02 (m, 2H), 7.60-7.57 (m, 1H), 7.51-7.45 (m, 2H), 5.21 (m, 1H), 2.4-1.0 (m, 32H), 1.15 (d, 3H, J=7 Hz), 1.09 (s, 3H), 0.91 (d, 3H, J=7 Hz), 0.67 (s, 3H); MS (ES+) 415.52 (M-C₇H₇O₂+H).

Preparation of Compound VI:

Compound VI may be prepared from 4, as shown in Scheme 1, using the methods described below for the synthesis of ENT-03, but instead of spermine being used during the reductive amination, a protected spermidine is instead used. As shown in Scheme 1, 4 may undergo reductive amination with azidospermidine (B) to produce Compound 7, using the method described below for the synthesis of 5 and substituting B for spermine. Compound7 may then be used to synthesize Compound VI via deprotection of the 7-hydroxyl group to produce Compound VI, using the procedure described below for the synthesis of ENT-03.

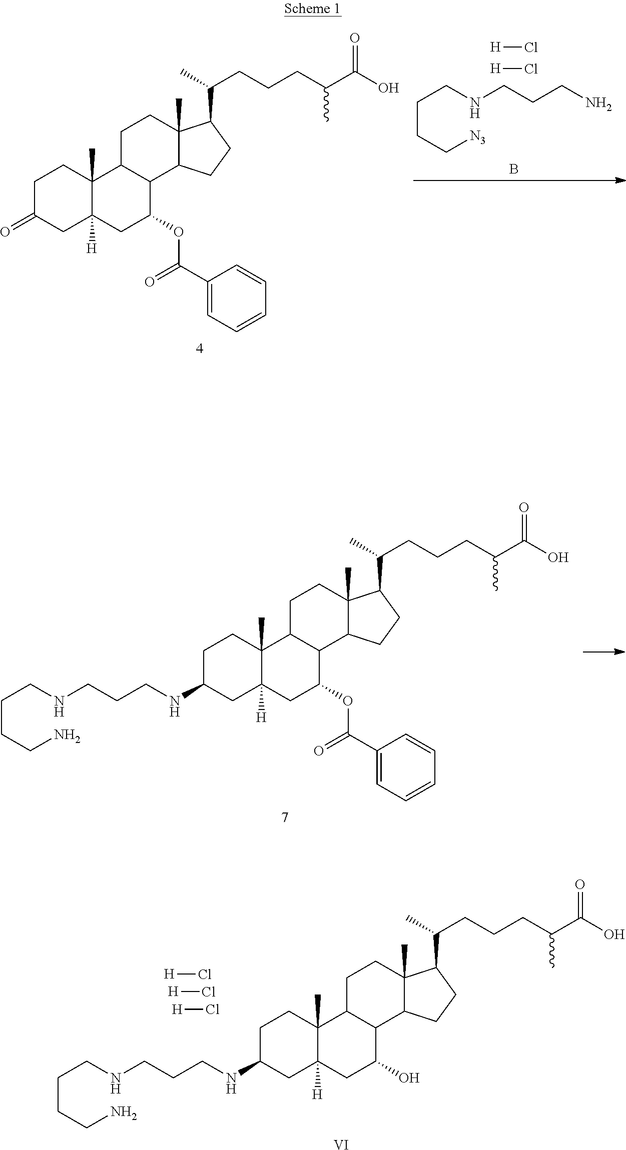

Preparation of Compound 5:

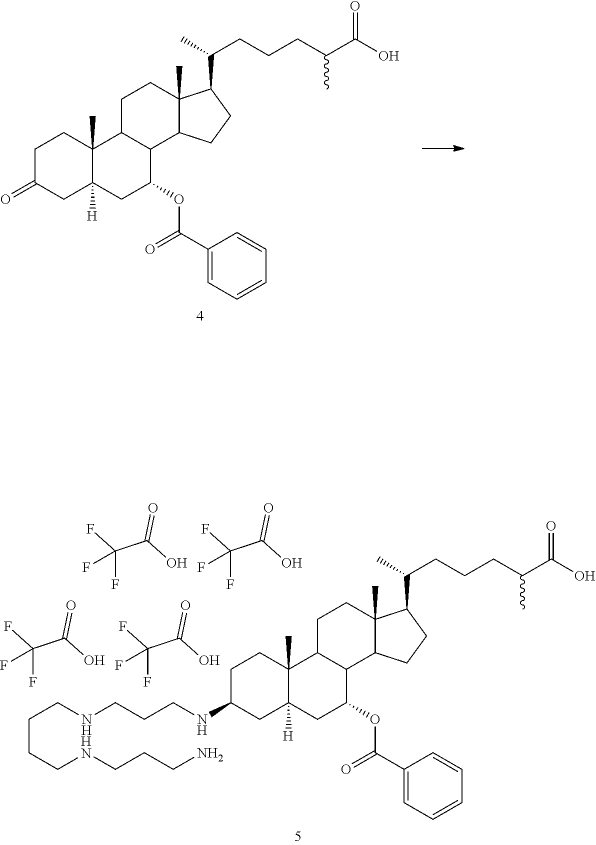

Compound 4 (3.5 g, 6.5 mmol) was dissolved in methanol (100 mL) and treated with spermine (5 g, 24.8 mmol) in methanol (˜10 mL). The mixture was stirred for 2 h at RT after which 2-propanol (100 mL) was added, and the majority of the solvent was removed in vacuo. The residue was redissolved in methanol (200 mL) and stirred overnight. Isopropyl alcohol (200 mL) was added and the mixture was evaporated to a thick residue. The residue was dissolved in anhydrous methanol (200 mL), and the solution chilled in a dry ice acetone bath under N₂ with vigorous magnetic stirring. When the internal temperature reached ˜−74° C., NaBH₄ (1.89 g, 50 mmol) was added. The temperature was maintained with the dry ice acetone bath for ˜4 h and then allowed to come to RT overnight. The reaction mixture was carefully acidified with 10% trifluoroacetic acid in water until pH paper showed pH 2-3 range. Water was added to the mixture, and the mixture was transferred to an oversized flask (to allow for frothing of the mixture on rotary evaporation) and the majority of the methanol removed in vacuo. The resulting solution was applied directly to amberchrome and eluted with a step gradient of acetonitrile in water with 0.5% TFA (10% increments 500 mL per increment) until aminosterol eluted (˜60% acetonitrile). The gradient was held at this point until all of the aminosterol eluted.

The fractions containing aminosterol were analyzed and the relatively clean fractions pooled and lyophilized to afford Compound 5 as the tetra-TFA salt (˜4.5 g, 3.8 mmol) of sufficient purity to carry on without further purification,

¹H NMR (CD₃OD, 300 MHz) δ 8.05-8.02 (m, 2H), 7.66-7.60 (m, 1H), 7.54-7.49 (m, 2H), 5.17 (m, 1H), 3.36-3.04 (m, 13H), 2.37 (m, 1H), 2.1-1.0 (m, 39H), 1.10 (d, 3H, J=7 Hz), 0.95 (m, 6H), 0.74 (s, 3H); MS (ES+) 723.78 (M+H).

Preparation of ENT-03:

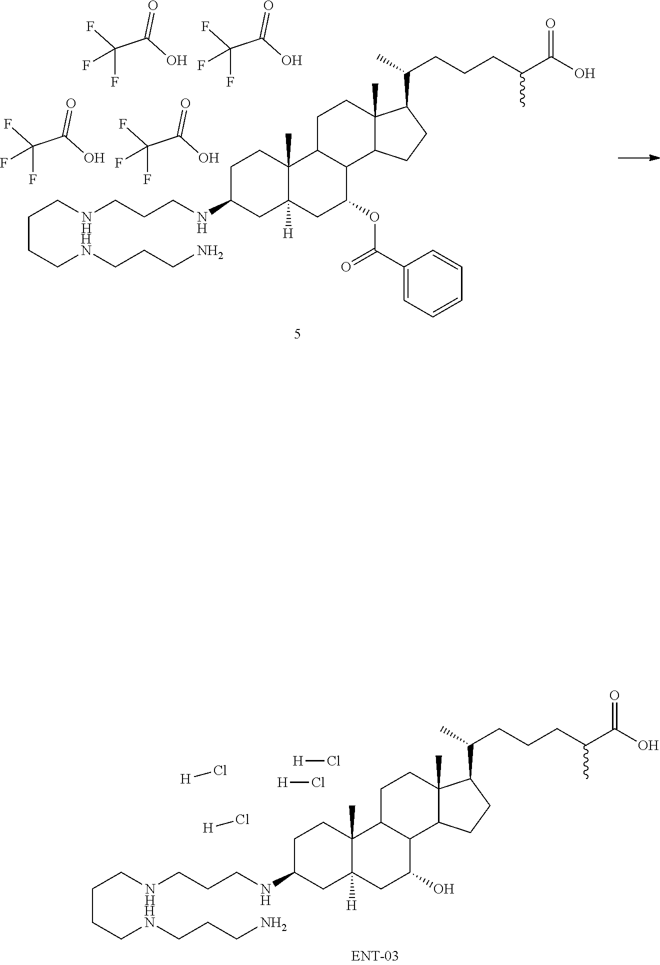

Compound 5 (3.0 g, 2.5 mmol) was added to of 5% methanolic potassium hydroxide (40 mL), and the solution was stirred for 2 days under N₂ at 110° C., and monitored with TLC (6:3:1 Chloroform, Methanol, conc. NH₄OH). After 2 days, the reaction was cooled to room temperature, evaporated under vacuum, and dissolved in H₂O (40 mL). This solution was then acidified with 6M HCl, and the white precipitate was forced back into solution with gentle heat and stirring. The solution was poured onto a large column of Amberchrome, and washed with H₂O (350 mL), increments (500 mL) of 10%, 15%, and 25% acetonitrile/water. Almost as soon as one column volume of 25% solution had passed through, the compound began to elute. The fractions (40 mL) that were collected were analyzed via LC/MS to separate away the 3-α side-product, which came off the column immediately following the desired 3-β product. Although there was some co-elution, a significant portion of the material came off cleanly.

These fractions were combined and lyophilized overnight to give (1.31 g, 1.7 mmol, 68%) of ENT-03 (Compound III) as the tetra-HCl salt,

¹H NMR (CD₃OD, 300 MHz) δ 3.80 (br s, 1H), 3.20-3.05 (m, 13H), 2.37 (m, 1H), 2.2-1.0 (m, 36H), 1.13 (d, 3H, J=7 Hz), 0.93 (d, 3H, J=7 Hz), 0.87 (s, 3H), 0.69 (s, 3H); MS (ES+) 619.31 (M+H).

Preparation of Compound 7:

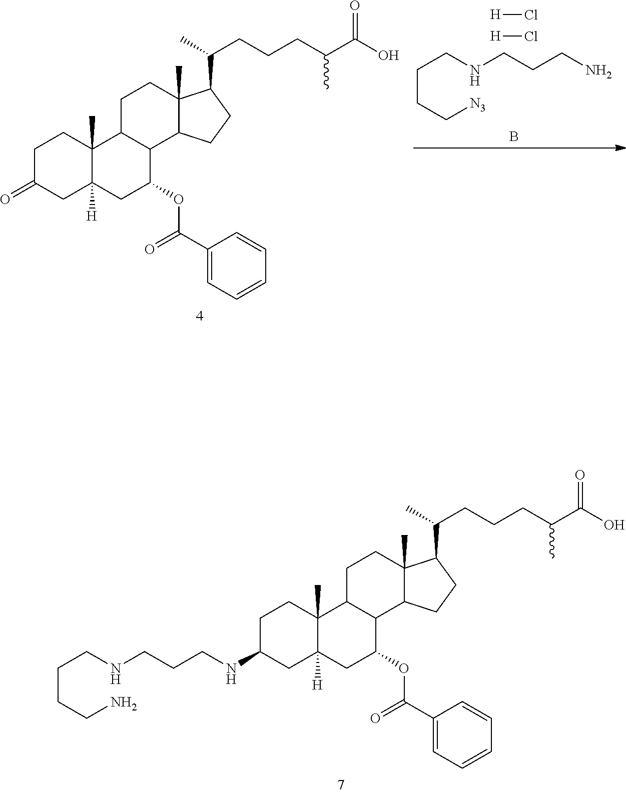

Azidospermidine di-HCl (B, 4.0 g, 16 mmol) was dissolved in methanol (50 mL) and potassium tert-butoxide (1.68 g, 15 mmol) of was added, and the mixture was stirred at 40° C. for 20 min. The resulting insoluble potassium chloride salt was filtered off, and the mother liquor was evaporated under vacuum. The resulting freebase azido-spermidine residue was dissolved in a 1:1:1 solution of methanol/isopropanol/pyridine (50 mL) and Compound 4 (2.3 g, 4.3 mmol) was added. The solution was stirred for 30 min, then stripped down under heat and vacuum to azeotrope away traces of water. The residue was re-dissolved and evaporated down in the same manner two more times. Finally, the imine residue was dissolved in 50 mL of anhydrous methanol and cooled down to −78° C. Sodium borohydride (1.0 gram, 26 mmol) was added in small portions over the course of 1 h. The solution was left to stir for an additional 2.5 h, then brought up to room temperature. TLC (20% IPA/Toluene) showed that no ketone remained, and no 3-OH side product appeared to have formed. The remaining borohydride was quenched with water, and two thirds of the methanol was evaporated. The mixture was poured onto Amberchrome, the salts washed away with water, and then the product was washed off the column with methanol. This fraction was concentrated down to 500 mL, then hydrogenated overnight at 45 psi with Raney Nickle catalyst.

The resulting solution was filtered through Celite® and concentrated to give Compound 7 (2.9 g),

¹H NMR (CD₃OD, 300 MHz) δ 8.03 (d, 2H, J=8 Hz), 7.6 (m, 1H), 7.5 (m, 2H), 5.2 (m, 1H), 3.2-2.9 (m, 9H), 2.1-1.0 (m, 38H), 1.07 (d, 3H, J=7 Hz), 0.97 (s, 3H), 0.75 (s, 3H); MS (ES+) 666.74 (M+H).

Preparation of Compound VI:

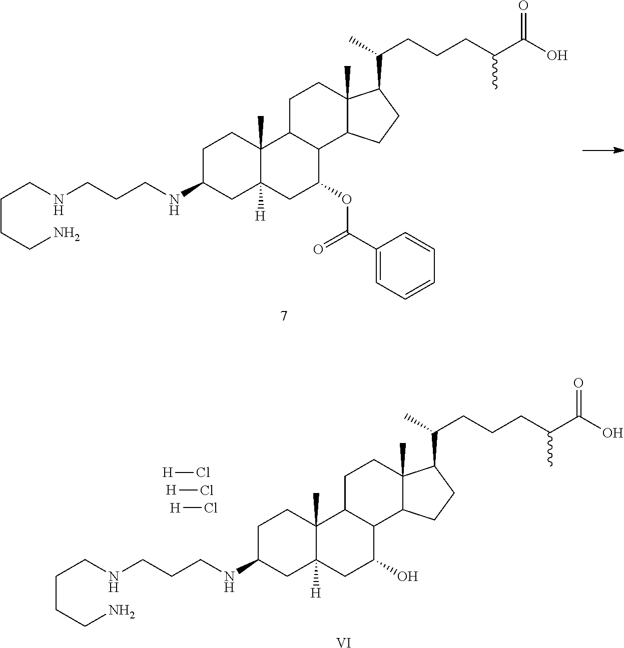

Compound 7 (2.9 g, ˜4.3 mmol) was dissolved in 6% methanolic potassium hydroxide (35 mL) and refluxed at 100° C. for 20 h. TLC analysis (6:3:1 chloroform/methanol/Ammonium hydroxide with Hannesian stain) indicated that none of the 7-benzoate material remained. Water (10 mL) was added, and the methanol was evaporated under vacuum. Additional water (40 mL) was added, and the solution was acidified with 6 M hydrochloric acid solution. This was poured onto a large column of Amberchrome, and washed with water (300 mL), 10% acetonitrile/water (400 mL), and then 25% acetonitrile/water (800 mL).

The appropriate fractions were collected, and lyophilized to afford Compound VI (0.95 g, 1.4 mmol, 33% two steps) of as the tri-HCl salt,

¹H NMR (CD₃OD, 300 MHz) δ 3.80 (br s, 1H), 3.21-2.97 (m, 9H), 2.40 (m, 1H), 2.2-1.0 (m, 35H), 1.14 (d, 3H, J=7 Hz), 0.94 (d, 3H, J=7 Hz), 0.88 (s, 3H), 0.70 (s, 3H); MS (ES+) 562.25 (M+H).

### Example 3: Treatment Induced Gene Expression in Mouse Stomach Tissues, Comparison of ENT-01 and ENT-06

The purpose of this example was to identify transcriptional changes between young and old mice and compare the effects of ENT-06 treatments on gene expression with those of ENT-01 (squalamine). Mice were treated with ENT-01, ENT-06 or a vehicle control. Samples of stomach tissues from the mice were analysed by RNA-sequencing on an Illumina platform.

To identify those genes that were significantly differentially expressed between groups, an arbitrary threshold was applied based on fold changes in expression. A four-fold change in expression between groups was used as a measure of pseudo-significance in the absence of replicate samples. At this threshold, differentially expressed genes were identified in all contrasts. For gene expression changes associated with ageing, or ENT-01 and ENT-02 (MSI-1436) treatments in young mice, the proportions of down-regulated genes (72-80%) were higher than the proportions of upregulated genes (20-28%). The trend was opposite for both treatments in old mice, where 82-89% of gene expression changes were up-regulation and 12-18% were down-regulation.

When comparing differentially expressed genes across contrasts, it was apparent that genes downregulated in aged mice overlapped significantly with genes up-regulated in response to ENT-01 treatment in old mice (hypergeometric P<0.0001). To a lesser extent, there was also significant overlap in genes down-regulated in aged mice and up-regulated in response to treatment in old mice. In young mice, genes that were down-regulated upon ENT-01 treatment significantly overlapped with the down-regulated genes associated with ageing. This result was also true for genes up-regulated in both contrasts.

For each comparison, the number of sample genes significant at various statistical thresholds and fold change 4 were tallied. As mentioned previously, for statistical robustness, only those with an adjusted p-value<0.05 should be considered.

The following statistical significance threshold was chosen to define differentially expressed genes: ·fold change≥4

TABLE 4
Summary of effect size evaluations: The number of significant features in each
comparison at a range of effect size thresholds. Each row relates to a single
comparison, while each column relates to a fold change threshold

| Contrast | Direction | Any | 1.3× | 2× | 4× | 8× | 16× |
|---|---|---|---|---|---|---|---|
| ENT-01 vs control (young) | Up-regulated | 7460 | 1430 | 232 | 58 | 38 | 28 |
| ENT-01 vs control (young) | Down-regulated | 7553 | 2367 | 642 | 144 | 47 | 12 |
| ENT-02 (MSI-1436) vs control (young) | Up-regulated | 7346 | 1860 | 232 | 30 | 7 | 1 |
| ENT-02 (MSI-1436) vs control (young) | Down-regulated | 7667 | 2429 | 531 | 120 | 43 | 16 |
| ENT-01 vs control (old) | Up-regulated | 7553 | 2430 | 766 | 317 | 168 | 116 |
| ENT-01 vs control (old) | Down-regulated | 7460 | 2235 | 431 | 66 | 25 | 11 |
| ENT-02 (MSI-1436) vs control (old) | Up-regulated | 7416 | 2109 | 657 | 298 | 139 | 42 |
| ENT-02 (MSI-1436) vs control (old) | Down-regulated | 7597 | 1395 | 203 | 38 | 7 | 1 |
| Old vs young (control) | Up-regulated | 7386 | 1715 | 312 | 76 | 28 | 11 |
| Old vs young (control) | Down-regulated | 7627 | 2486 | 734 | 218 | 85 | 39 |
| Old vs young (ENT-01) | Up-regulated | 7479 | 1647 | 559 | 263 | 178 | 135 |
| Old vs young (ENT-01) | Down-regulated | 7534 | 1606 | 267 | 69 | 36 | 27 |
| Old vs young (ENT-02 (MSI-1436)) | Up-regulated | 7669 | 2644 | 676 | 190 | 78 | 39 |
| Old vs young (ENT-02 (MSI-1436)) | Down-regulated | 7344 | 1986 | 314 | 63 | 8 | 4 |

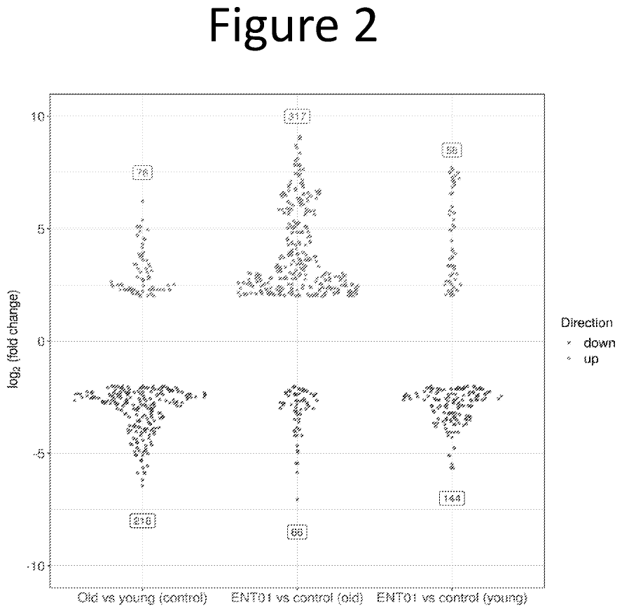

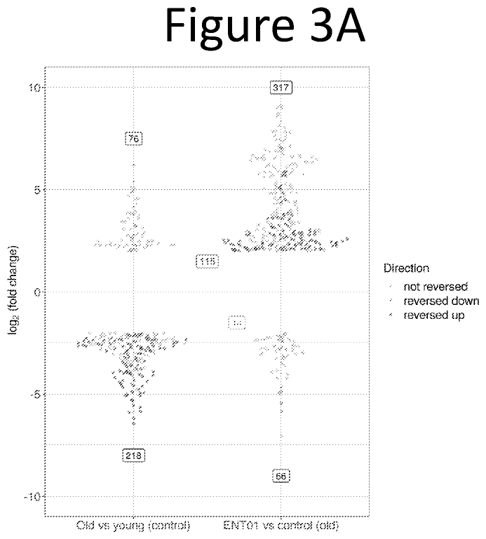

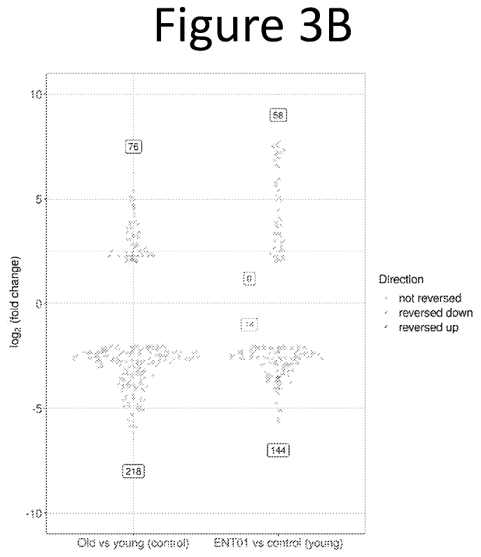

FIG. 2 shows transcriptional changes in response to ENT-01 in the stomach of old and young mice while FIG. 3 shows ageing associated gene expression changes reversed by ENT-01 treatment. In order to investigate the overlap between selected genes from the multiple contrasts performed, Fios Genomics counted the number of overlapping differentially expressed genes (defined using fold change 4) between all pairwise combinations of the comparisons performed. The amount of overlap is represented in FIG. 4A-4D. For each comparison, the value in the plot represents the number of intersecting selected genes and the colour represents the Jaccard index (the intersection over the union) for the two contrasts under consideration.

To investigate the overlap between significant genes from the contrasts performed, Fios Genomics generated a set of scatter plots comparing the fold change between pairs of contrasts. Functional enrichment analysis was performed upon those genes where expression differences were greater than four-fold in individual contrasts. Reactome and GO term databases were interrogated to identify relevant terms that were significantly enriched in differentially expressed genes (enrichment P<0.05). Pathways related to the immune system such as platelet degranulation, antimicrobial peptides and complement cascade were generally amongst the most enriched pathways across the contrasts. Pathways such as keratinisation and keratinocyte differentiation were enriched in genes changed upon ageing or ENT-01 treatments in old mice. Pathways such as muscle contractions and sarcomere organisation were enriched in genes changed upon ageing, but were also enriched in ENT-01 treatment-affected genes in young mice.

Significant genes (at fold change 4) from each contrast were analysed for enrichment of Reactome pathway membership using a hypergeometric test by mapping genes to genes (if appropriate). Enrichment (p-value<0.05) was assessed for the union of selected genes.

Genes that were differentially expressed in response to ENT-01 treatment, as identified in this study, were compared to those that were associated with ENT-06 treatments, respectively. ENT-06 treatment-associated gene expression changes were identified previously. Across all contrasts at the relevant statistical thresholds used to identify significant features, there were ten or less genes that changed in expression upon both treatments (ENT-01 and ENT-06). Note that this included the gene expression changes associated with ageing. Specifically, the expression of only one gene, Sypl2, was changed significantly between the old and young mice of both studies.

Comparisons of the genes which were down-regulated in ageing and up-regulated in treatment revealed that genes affected by ENT-01-treatment did not overlap with those affected by ENT-06.

Congruence analysis was performed between the contrasts Specifically, the analysis compared the effects of human- or shark-origin compound analogues on murine stomach tissue gene expression profile as follows: ENT-01 was compared to ENT-06. Scatter plots, upset plots, venn diagrams, hypergeometric tests and Spearman rank correlation tests were employed to assess the level of overlap. Note that significantly differentially expressed genes in ENT-06-specific contrasts were determined using a statistical threshold of FDR-adjusted P<0.05. Whereas the ENT-01 contrasts in this study defined significant genes using a cutoff of greater than four-fold change in expression.

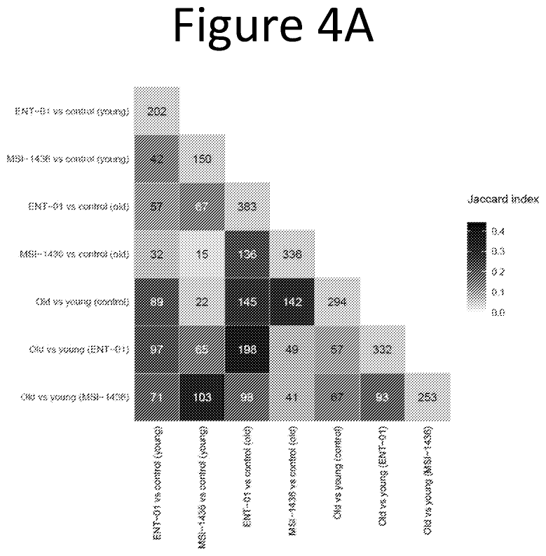

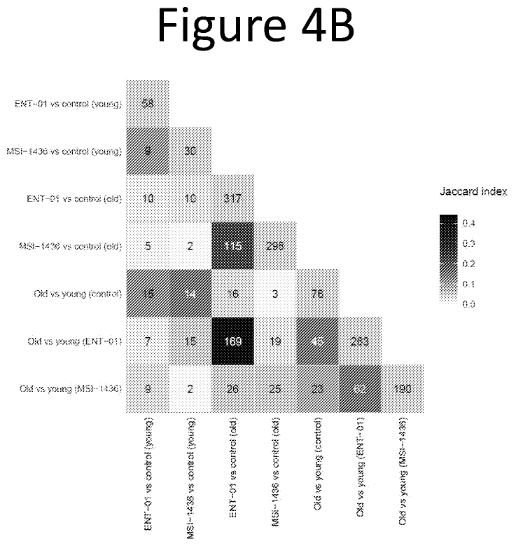

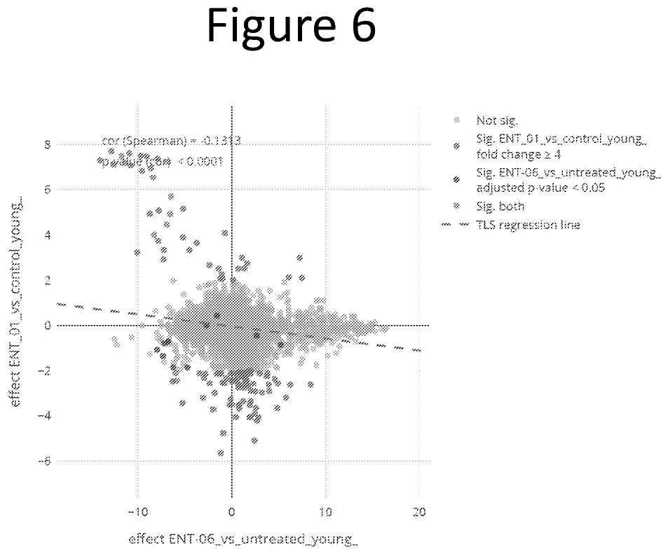

Transcripts that were significantly down-regulated in ageing and up-regulated by treatment of old mice were identified for each compound (See FIG. 5). Congruence between these sets of genes was then assessed by comparing ENT-01-affected genes to those of ENT-06. FIG. 6 shows a scatter plot comparing significant genes in ENT-01 vs control (young) against ENT-06 vs untreated (young). FIG. 8 shows Venn diagrams of significant genes in ENT-01 vs control (young) against ENT-06 vs untreated (young). Each plot considers a different interaction of sets; either ignoring direction of perturbation, considering only up-regulated genes, considering only down-regulated genes, or examining the overlap between those genes up-regulated in one contrast and those genes down-regulated in another. The symbol U denotes the universe. Overlap was assessed between ENT-01 vs control (young) (202 genes) and ENT-06 vs untreated (young) (26 genes). 10 genes overlapped between the two sets when not considering direction of change. This is significantly more than would be expected by chance (p=1.721e−16). The Spearman rank correlation between the genes in the two sets was −0.1313. This is significantly different to zero (p=5.678e−57).

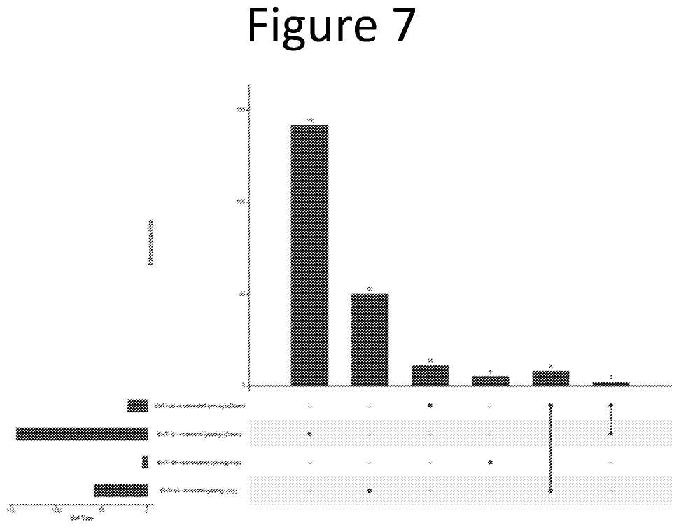

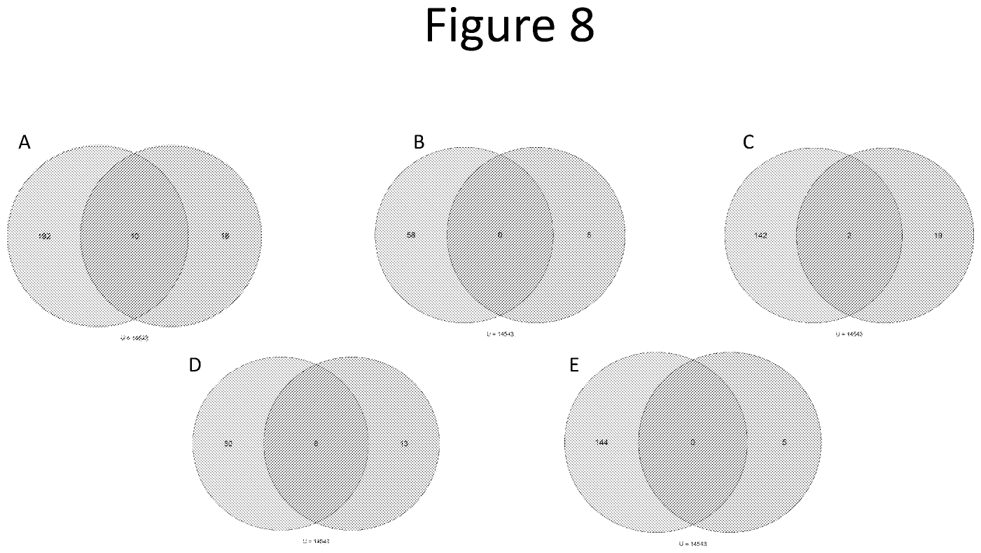

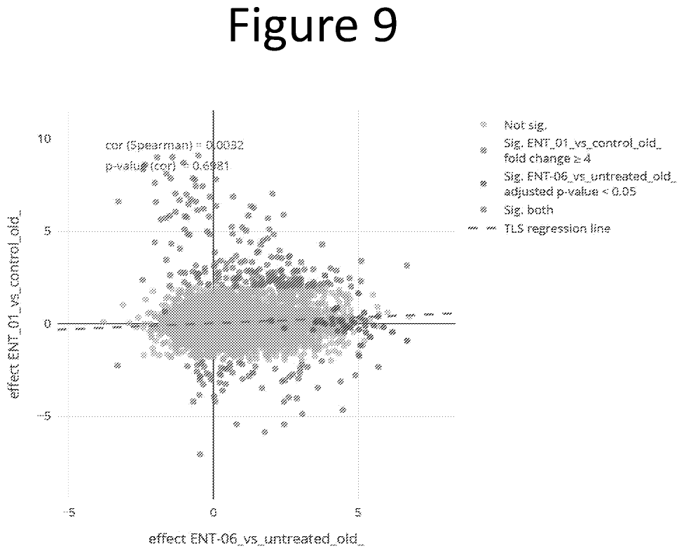

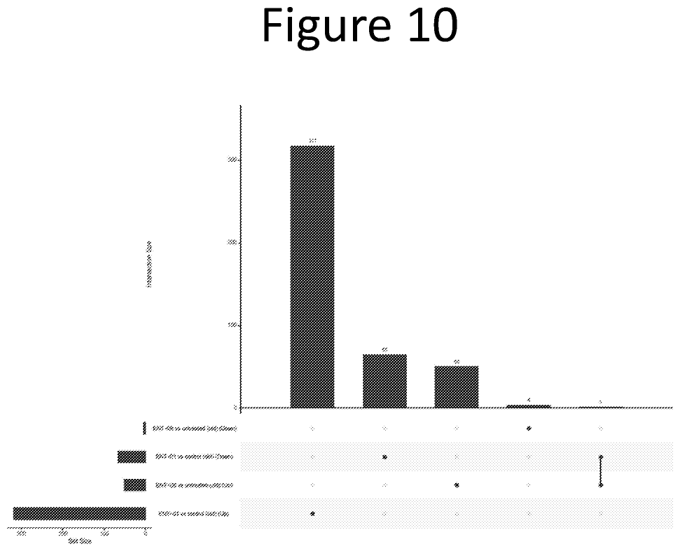

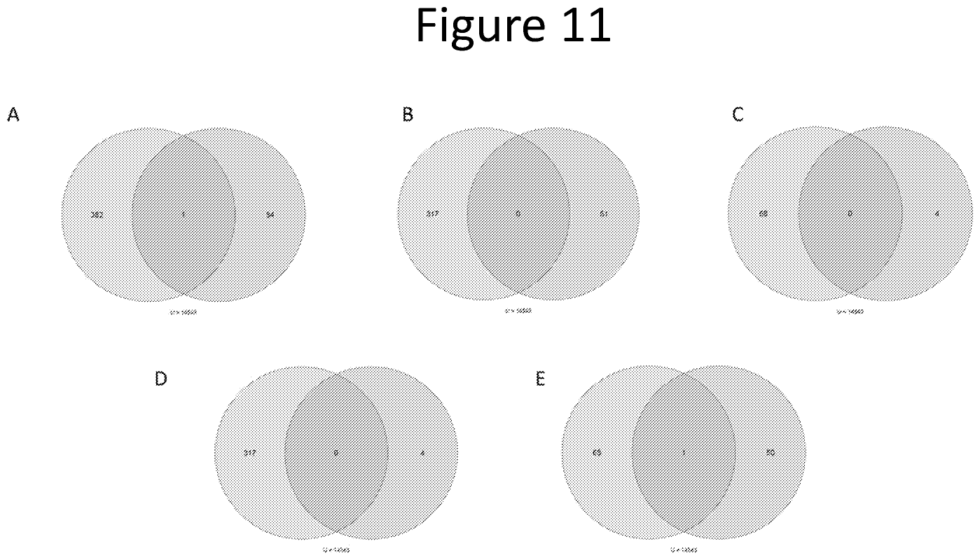

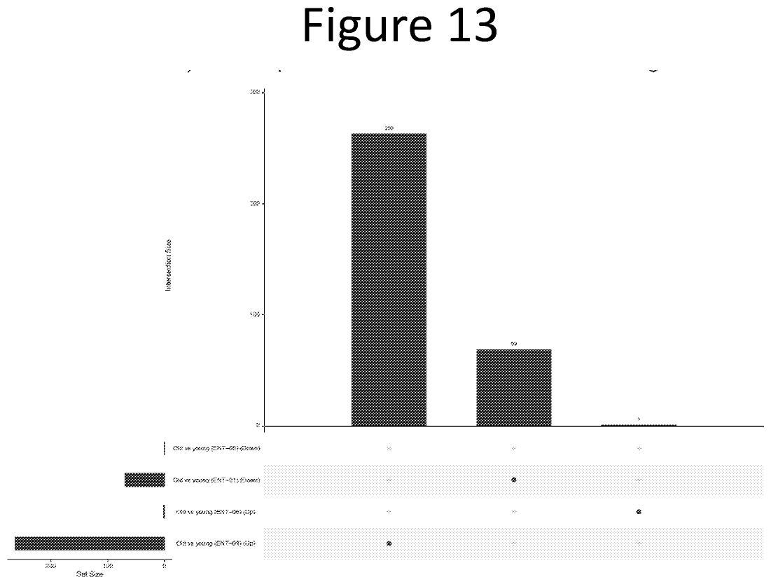

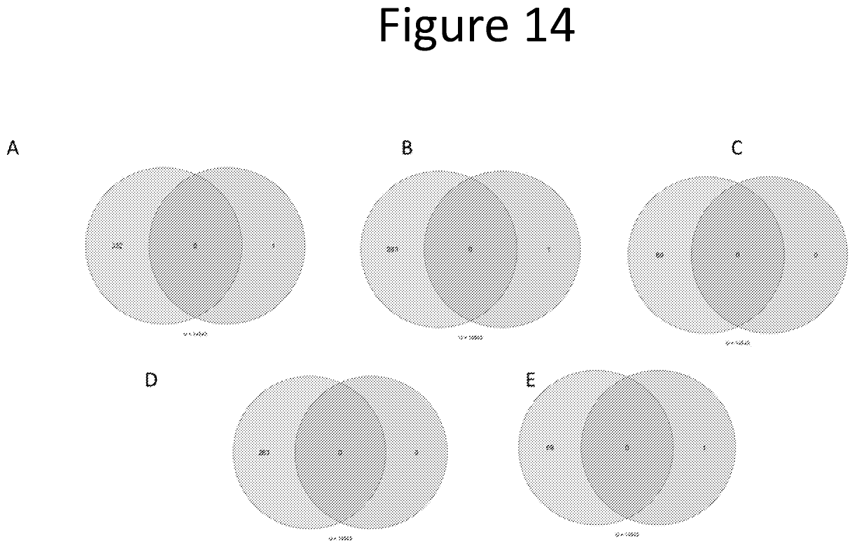

Congruence analysis for: ENT-01 vs ENT-06 (old) was also conducted. FIG. 9 shows a scatter plot comparing significant genes in ENT-01 vs control (old) against ENT-06 vs untreated (old). FIG. 10 shows the interactions between sets of up and down regulated genes. FIG. 11 shows Venn diagrams of significant genes in ENT-01 vs control (old) against ENT-06 vs untreated (old). Overlap was assessed between ENT-01 vs control (old) (383 genes) and ENT-06 vs untreated (old) (55 genes). 1 genes overlapped between the two sets when not considering direction of change. This is no more than would be expected by chance (p=0.5057). The Spearman rank correlation between the genes in the two sets was 0.003217. Congruence analysis of Old vs young (ENT-01 vs ENT-06) was conducted and the results are shown in FIG. 12. FIG. 14 shows Venn diagrams of significant genes in Old vs young (ENT-01) against Old vs young (ENT-06).

### Example 4: Cognitive Impairment

This prophetic example describes an exemplary method of (i) treating cognitive impairment and/or (ii) treating and/or preventing a disorder in which cognitive impairment is a known symptom (a cognitive impairment related disorder) in a subject having cognitive impairment.

Patients are selected based on having cognitive impairment. Patients are grouped based on having a particular cognitive impairment associated disorder or having cognitive impairment with no underlying disorder. The groups are then subdivided into a control subgroup and a treatment subgroup. An oral dose of an aminosterol or of a salt or derivative thereof between 50-500 mg per day is administered for each of the patients in the treatment subgroup until an improvement in one or more of the following tests is observed: ADASCog, Woodcock-Johnson Tests of Cognitive Abilities, Leiter International Performance Scale, Miller Analogies Test, Raven's Progressive Matrices, Wonderlic Personnel Test, IQ tests, Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA),

Patients are selected based on having hallucinations. Patients are grouped based on having a particular hallucination-associated disorder or having hallucinations with no underlying disorder. The groups are then subdivided into a control subgroup and a treatment subgroup. An intranasal dose of about 0.001 mg up to about 6 mg/day of an aminosterol or a salt or derivative thereof is administered for each of the patients in the treatment subgroup using the improvement of hallucination symptoms as an endpoint. Patients are monitored for changes in the severity or occurrence of the symptoms. Patients with an underlying disorder are also monitored for changes in other symptoms associated with the disorder. Patients with no underlying disorder are monitored for the development of a hallucination associated disorder. The improvement in hallucination symptoms is monitored using one or more tests collected from: The University of Miami Parkinson's Disease Hallucinations Questionnaire (UM-PDHQ), Unified Parkinson's Disease Scale (UPSRS), section 1.2 (Hallucinations and Psychosis), direct questioning, Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

### Example 5: Schizophrenia

This prophetic example describes an exemplary method of treating and/or preventing schizophrenia in a subject in need thereof.

Patients are selected based on being diagnosed with schizophrenia, i.e., having schizophrenia, or exhibiting known risk factors for schizophrenia, i.e., at risk for developing schizophrenia. Patients are grouped based on having schizophrenia or at risk for developing schizophrenia. The groups are then subdivided into a control subgroup and a treatment subgroup. An intranasal dose of about 0.001 mg up to about 6 mg/day of an aminosterol or a salt or derivative thereof is administered for each of the patients in the treatment subgroup using the improvement constipation or another symptom of schizophrenia as an endpoint. Patients are monitored for changes in the severity or occurrence of the symptoms. Patients having schizophrenia are monitored for changes in other symptoms associated with the disorder. Patients at risk for developing schizophrenia are monitored for the development of schizophrenia. Changes in symptoms associated with schizophrenia may be assessed using one or more tests collected from Positive and Negative Syndrome Scale (PANSS), the Psychotic Symptom Rating Scales (PSYRATS), the Quality of Life Scale (QLS), the Schizophrenia Cognition Rating Scale (SCoRS), the Drug Attitude Inventory (DAI), and the Abnormal Involuntary Movement Scale (AIMS).

While certain embodiments have been illustrated and described, it should be understood that changes and modifications can be made therein in accordance with ordinary skill in the art without departing from the technology in its broader aspects as defined in the following claims.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Aarsland et al., “Neuropsychiatric symptoms in patients with Parkinson's disease and dementia: frequency, profile and associated care giver stress,” J. Neurol. Neurosurg. Psychiatry, 78:36-42 (2007).

Acsadi et al., “Alpha-synuclein loss in spinal muscular atrophy,” J. Mol. Neurosci., 43(3):275-83 (2011).

Ahima RS, Patel HR, Takahashi N, Qi Y, Hileman SM, Zasloff MA. “Appetite suppression and weight reduction by a centrally active aminosterol,” Diabetes, 51(7):2099-104 (2002).

Akhter, S., S. K. Nath, et al. (1999). “Squalamine, a novel cationic steroid, specifically inhibits the brush-border Na+/H+exchanger isoform NHE3.” Am J Physiol 276(1 Pt 1): C136-44.

Andresen, et al., “Effect of 5 Days Linaclotide on Transit and Bowel Function in Females With Constipation-Predominant Irritable Bowel Syndrome,” Gastroenterology, Volume 133, Issue 3, September 2007, Pages 761-768.

Antonelou et al., “Decreased levels of alpha-synuclein in cerebrospinal fluid of patients with clinically isolated syndrome and multiple sclerosis,” J. Neurochem., 134(4):748-55 (2015).

Antonio-Rubio, et al., “Abnormal thermography in Parkinson's disease,” Parkinsonism Relat Disord., 2015 August;21(8):852-7.

Auyeung et al., “Ten year survival and outcomes in a prospective cohort of new onset Chinese Parkinson's disease patients,” J. Neurol. Neurosurg. Psychiatry, 83:607-11 (2012).

Beach et al Neurol Ther. 2017 July; 6(Suppl 1): 5-13

Berg et al., “MDS Research Criteria for Prodromal Parkinson's Disease,” Mov. Disord., 30:1600-1611 (2015).

Bhargava et al., “A phase I and pharmacokinetic study of squalamine, a novel antiangiogenic agent, in patients with advanced cancers,” Clin. Cancer Res., 7:3912-9 (2001).

Boeve et al., “Synucleinopathy pathology and REM sleep behavior disorder plus dementia or parkinsonism,” Neurology 61(1):40-5 (July 2003).

(a) Braak et al., “Idiopathic Parkinson's disease: possible routes by which vulnerable neuronal types may be subject to neuroinvasion by an unknown pathogen,” J. Neural. Transm. (Vienna), 110:517-36 (2003).

(b) Braak et al., “Staging of brain pathology related to sporadic Parkinson's disease,” Neurobiol. Aging, 24:197-211 (2003).

Breen, et al., “Sleep and circadian rhythm regulation in early Parkinson disease,” JAMA Neurol., 2014 May; 71(5):589-595.

Brundin, et al., “Prying into the Prion Hypothesis for Parkinson's Disease,” J Neurosci., 2017 Oct. 11;37(41):9808-9818.

Brunel et al., “Squalamine: A Polyvalent Drug of the Future?” Current Cancer Drug Targets, 5(4):267-272(6) (2005)

Claassen et al., “REM sleep behavior disorder preceding other aspects of synucleinopathies by up to half a century,” Neurology, 75(6):494-499 (August 2010).

Combs AP, “Recent advances in the discovery of competitive protein tyrosine phosphatase 1B inhibitors for the treatment of diabetes, obesity, and cancer” J. Med. Chem., 53(6):2333-44 (2010)

Connolly, et al., “Squalamine Lactate for Exudative Age-Related Macular Degeneration,” Ophthalmology Clinics of North America, 19, 381-391 (2006).

Corrochano et al., “α-synuclein levels modulate Huntington's disease in mice,” Hum. Mol. Genet., 21(3):485-94 (February 2012).

Demirel et al., “Decreased Expression of α-Synuclein, Nogo-A and UCH-L1 in Patients with Schizophrenia: A Preliminary Serum Study,” Psychiatry Invest., 14(3) (2017).

Diao, et al., “Human Kidney is a Target for Novel Severe Acute Respiratory Syndrome Coronavirus 2 (SARS-CoV-2) Infection” 2020 medRxiv Preprint. Posted Apr. 10, 2020.

Diederich et al., “Hallucinations in Parkinson disease,” Nat. Rev. Neurol., 5:331-42 (2006).

Elchebly et al., “Increased insulin sensitivity and obesity resistance in mice lacking the protein tyrosine phosphatase-1B gene” Science. 1999 Mar. 5;283(5407):1544-8.

Fahn S ER, Members of the UPDRS Development Committee. UNIFIED PARKINSON'S DISEASE RATING SCALE. Florham Park, N.J.: Macmillan Health Care Information (1987).

Frank, et al., “Psychometric validation of a constipation symptom assessment questionnaire,” Scand J Gastroenterol., 1999 September;34(9):870-7.

Frisinia, et al., “The neuropathological basis for depression in Parkinson's disease,” Parkinsonism Relat Disord., 2009 February; 15(2): 144-148.

Genaidy, et al., “Effect of Squalamine on Iris Neovascularization in Monkeys,” Retina, December 2002—Volume 22—Issue 6-p 772-778.

Gjerstad et al., “Excessive daytime sleepiness in Parkinson disease: is it the drugs or the disease?” Neurology, 67:853-8 (2006).

Goetz, et al., “Risk factors for nursing home placement in advanced Parkinson's disease,” Neurology, 43:2227-9 (1993).

Hao et al., “A Phase I and pharmacokinetic study of squalamine, an aminosterol angiogenesis inhibitor,” Clin. Cancer Res., 9:2465-71 (2003).

Heaton, et al., “Defecation frequency and timing, and stool form in the general population: a prospective study,” Gut., 1992 June; 33(6): 818-824.

Herbst, Roy S. et al., “A Phase/IIA Trial of Continuous Five-Day Infusion of Squalamine Lactate (MSI-1256F) Plus Carboplatin and Paclitaxel in Patients with Advanced Non-Small Cell Lung Cancer,” Clin. Cancer Res. 9(11) (2003).

Higgins, et al., “Regression of Retinopathy by Squalamine in a Mouse Model,” Pediatric Research, volume 56, pages 144-149 (2004).

Higgins, et al., “Squalamine Improves Retinal Neovascularization,” Investigative Ophthalmology & Visual Science, May 2000, Vol. 41, 1507-1512.

Holmqvist, et al., “Direct evidence of Parkinson pathology spread from the gastrointestinal tract to the brain in rats,” Acta neuropathological, 128. 10.1007/s00401-014-1343-6 (2014).

Hosokawa et al., “Accumulation of multiple neurodegenerative disease-related proteins in familial frontotemporal lobar degeneration associated with granulin mutation,” Scientific Reports, 7:1513 (2017).

Hughes, et al., “What features improve the accuracy of clinical diagnosis in Parkinson's disease,” Neurology, Jun. 1, 1992; 42 (6).

Kim et al., “Poststroke Induction of α-Synuclein Mediates Ischemic Brain Damage,” J. Neurosci., 36(26):7055-65 (2016).

Krishnan et al., “A potent, selective, and orally bioavailable inhibitor of the protein-tyrosine phosphatase PTP1B improves insulin and leptin signaling in animal models,” J. Biol. Chem., 293(5):1517-1525 (Feb. 2, 2018).

Lantz, et al., “Inhibition of PTP1B by trodusquemine (MSI-1436) causes fat-specific weight loss in diet-induced obese mice.” Obesity (Silver Spring). 2010 August; 18(8):1516-23.

Lewis, et al., “Stool form scale as a useful guide to intestinal transit time,”. Scand J Gastroenterol., 1997; 32:920-924.

Lin, et al., “Genetics and genomics of Parkinson's disease,” Genome Medicine, 2014 6:48.

MacDonald, D. (1995). “Squalamine for STDs.” Abstract no F7 35th ICAAC conference.

Madrid-Navarro, et al., “Multidimensional Circadian Monitoring by Wearable Biosensors in Parkinson's Disease,” FrontNeurol., 2018; 9: 157.

Magen et al., “Mouse Models of Cognitive Deficits Due to Alpha-Synuclein Pathology,” J. of Parkinson's Dis., 1:217-227 (2011).

Marquis, et al., “Development and validation of the Patient Assessment of Constipation Quality of Life questionnaire,” Scand J Gastroenterol., 2005 May; 40(5):540-51.

McDowell et al., “Sleep dysfunction and EEG alterations in mice overexpressing alpha-synuclein,” J. Parkinsons Dis., 4(3):531-539 (2014).

McKhann, et al., “The diagnosis of dementia due to Alzheimer's disease: recommendations from the National Institute on Aging-Alzheimer's Association workgroups on diagnostic guidelines for Alzheimer's disease,” Alzheimers Dement., 2011 May; 7(3):263-9.

Mearin et al., “Bowel Disorders,” Gastroenterology, 150(6):1393-1407 (2016).

Morairty, “Detecting Neurodegenerative Diseases Before Damage Is Done,” SRI International (Jul. 26, 2013).

Moussaud et al., “Alpha-synuclein and tau: teammates in neurodegeneration?” Mol. Neurodeg., 9:43 (2014).

Ondo et al., “Daytime sleepiness and other sleep disorders in Parkinson's disease,” Neurology, 57:1392-6 (2001).

Ondo, et al., “Placebo-controlled trial of lubiprostone for constipation associated with Parkinson disease,” Neurology, 2012 May 22;78(21):1650-4.

Ortiz-Tudela et al., “Ambulatory circadian monitoring (ACM) based on thermometry, motor activity and body position (TAP): a comparison with polysomnography,” Physiol. Behav., 126:30-8 (2014).

Palma et al., “Treatment of autonomic dysfunction in Parkinson disease and other synucleinopathies”. Mov. Disord. (Review), 33(3):372-90 (March 2018).

Palsetia et al., “The Clock Drawing Test versus Mini-mental Status Examination as a Screening Tool for Dementia: A Clinical Comparison,” Indian J. Psychol. Med., 40:1-10 (2018).

Papapetropoulos et al., “A questionnaire-based (UM-PDHQ) study of hallucinations in Parkinson's disease,” BMC Neurol., 8:21 (2008).

Pelkmans, L. and A. Helenius (2003). “Insider information: what viruses tell us about endocytosis.” Curr Opin CellBiol 15(4): 414-22.

Phillips, et al., “Alpha-synuclein-immunopositive myenteric neurons and vagal preganglionic terminals: autonomic pathway implicated in Parkinson's disease?,” Neuroscience. 2008 May 15;153(3):733-50.

Poyiadji et al., COVID-19-associated “Acute Hemorrhagic Necrotizing Encephalopathy: CT and MRI Features” Radiology. Published Online:Mar. 31 2020

Rocca et al., “The Role of T1-Weighted Derived Measures of Neurodegeneration for Assessing Disability Progression in Multiple Sclerosis,” Front Neurol., 8:433 (Sep. 4, 2017).

Salmi et al., “Squalamine: An Appropriate Strategy against the Emergence of Multidrug Resistant Gram-Negative Bacteria?” PLoS One. 3(7):e2765 (2008).

Sarabia et al., “Circadian rhythm of wrist temperature in normal-living subjects A candidate of new index of the circadian system,” Physiol. Behav., 95:570-80 (2008).

Schiller, JH and G. Bittner, “Potentiation of platinum antitumor effects in human lung tumor xenografts by the angiogenesis inhibitor squalamine: effects on tumor neovascularization,” Clin Cancer Res. 5(12):4287-94 (1999).

Shehata et al., “Neuronal stimulation induces autophagy in hippocampal neurons that is involved in AMPA receptor degradation after chemical long-term depression,” J. Neurosci., 32:10413-22 (2012).

Sills Jr., Allen K., et al., “Squalamine Inhibits Angiogenesis and Solid Tumor Growth in Vivo and Perturbs Embryonic Vasculature,” Cancer Research 58:2784-2792 (1998).

Sokoloff, et al., “Adjunctive therapy for men with high risk localized and locally advanced prostate cancer: targeting disseminated tumor cells,” J Urol., 2004 December;172(6 Pt 2):2539-44.

Sriwimol and Limprasert, “Significant Changes in Plasma Alpha-Synuclein and Beta-Synuclein Levels in Male Children with Autism Spectrum Disorder,” BioMed Research International, 2018, 7 pages (2018).

Steer et al., “Use of the Beck Depression Inventory-II with depressed geriatric inpatients,” Behav. Res. Ther., 38:311-8 (2000).

Stiasny-Kolster et al., “The REM sleep behavior disorder screening questionnaire—a new diagnostic instrument,” Movement disorders: Official J. of the Movement Dis. Soc., 22:2386-93 (2007).

Stoessl, “Neuroimaging in the early diagnosis of neurodegenerative disease,” Transl. Neurodegener., 1: 5 (2012).

Svensson, et al., “Vagotomy and subsequent risk of Parkinson's disease,” Ann. Neurol., 2015; 78:522-529.

The US Burden of Disease Collaborators, “The State of US Health, 1990-2016: Burden of Diseases, Injuries, and Risk Factors Among US States,” JAMA, 319(14):1444-1472 (2018)

Videnovic, et al., “Circadian Dysregulation in Parkinson's Disease,” Neurobiol Sleep Circadian Rhythms, 2017 January;2:53-58.

Wadman, Couzin-Frankel, Kaiser, Matacic “How does coronavirus kill? Clinicians trace a ferocious rampage through the body, from brain to toes,” Science (2020).

Williams, J I et al., “Squalamine treatment of human tumors in nu/nu mice enhances platinum-based chemotherapies,” Clin Cancer Res. (2001).

Wimo, et al., “The worldwide economic impact of dementia 2010,” Alzheimer's Dement., 9: 1-11 (2013).

Wu P, Duan F, Luo C, et al. Characteristics of Ocular Findings of Patients With Coronavirus Disease 2019 (COVID-19) in Hubei Province, China. JAMA Ophthalmol. Published online Mar. 31, 2020. doi:10.1001/jamaophthalmol.2020.1291

Yancopoulou et al., “Tau and alpha-synuclein inclusions in a case of familial frontotemporal dementia and progressive aphasia,” J. Neuropathol. Exp. Neurol., 64(3):245-53 (2005).

Yin, M., C. Gentili, et al. (2002). “Antiangiogenic treatment delays chondrocyte maturation and bone formation during limb skeletogenesis,” J. Bone Miner Res 17(1):56-65.

Zahodne, et al., “Components of Depression in Parkinson Disease,” J Geriatr Psychiatry Neurol., 2012 September; 25(3): 131-137.

Zasloff, et al., “A spermine-coupled cholesterol metabolite from the shark with potent appetite suppressant and antidiabetic properties,” Int J Obes Relat Metab Disord., 2001 May; 25(5):689-97.

Zhang, Chao et al. “Liver injury in COVID-19: management and challenges.” The lancet. Gastroenterology & hepatology vol. 5,5 (2020): 428-430. doi:10.1016/S2468-1253(20)30057-1.

Zhao et al., “A comparative study of the amount of α-synuclein in ischemic stroke and Parkinson's disease,” Neurol. Sci.,37(5):749-54 (2016).

Zinsmeister et al., “Pharmacodynamic and clinical endpoints for functional colonic disorders: statistical considerations,” Dig. Dis. Sci., 58:509-18 (2013).

1. An aminosterol compound having the formula: 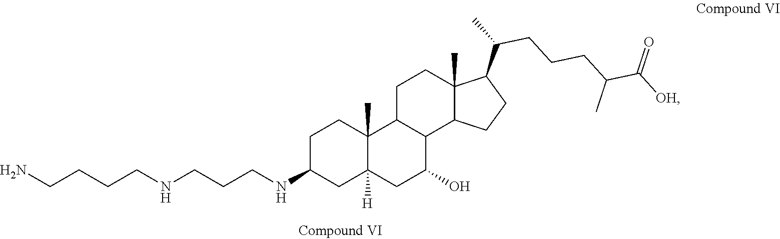 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]
or a pharmaceutically acceptable salt, solvate, prodrug, or derivative thereof.

2. The aminosterol compound of claim 1:
(a) having the formula: 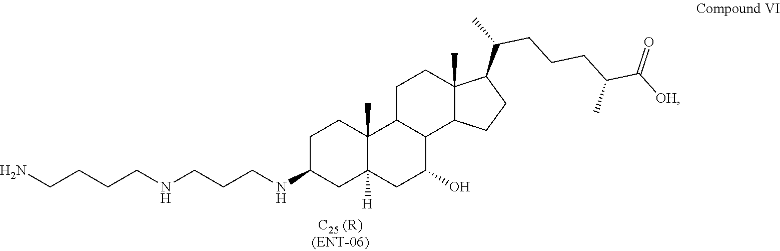 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]
or a pharmaceutically acceptable salt, solvate, prodrug, or derivative thereof; or
(b) having the formula: 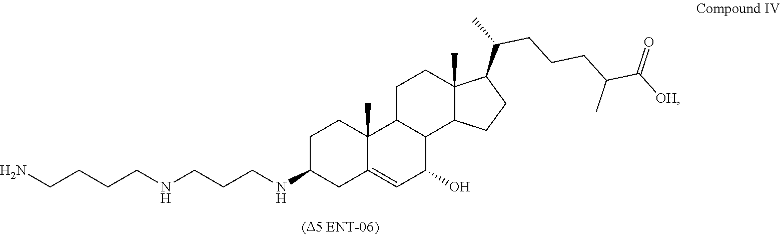 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]
or a pharmaceutically acceptable salt, solvate, prodrug, or derivative thereof;
(c) having the formula: 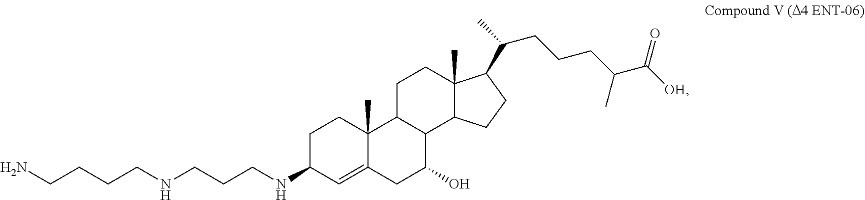 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]
or a pharmaceutically acceptable salt, solvate, prodrug, or derivative thereof; or
(d) having the formula: 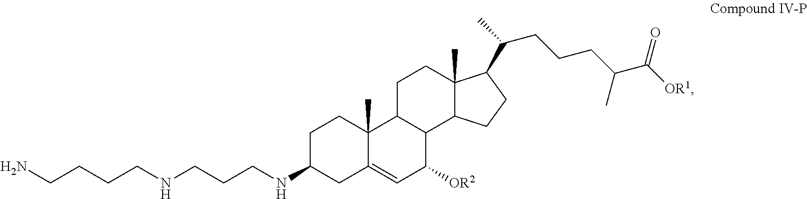
wherein:
R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and
R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl;
provided that at least one of R¹ and R² is not H,
or a pharmaceutically acceptable salt, solvate, prodrug, or derivative thereof; or
(e) having the formula: 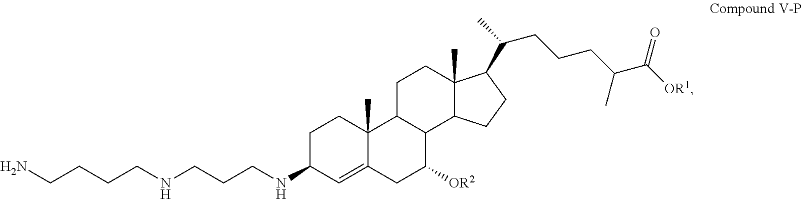
wherein:
R¹ is H, an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, and optionally substituted C₁-C₆ alkenyl; and
R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl;
provided that at least one of R¹ and R² is not H, or
a pharmaceutically acceptable salt, solvate, prodrug, or derivative thereof; or
(f) having the formula: 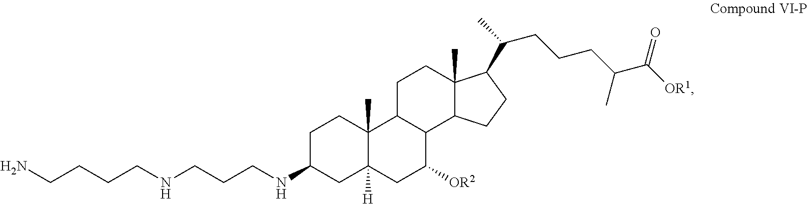
wherein:
R¹ is H, optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl; and
R² is H or —C(O)R³, wherein R³ is an optionally substituted aryl, optionally substituted heteroaryl, optionally substituted C₁-C₆ alkyl, optionally substituted C₁-C₆ alkynyl, optionally substituted heterocyclyl, optionally substituted C₃-C₈ cycloalkyl, or optionally substituted C₁-C₆ alkenyl;
provided that at least one of R¹ and R² is not H, or
a pharmaceutically acceptable salt, solvate, prodrug, or derivative thereof.

3. (canceled)

4. The aminosterol compound:
(a) of claim 2(b) having the formula: 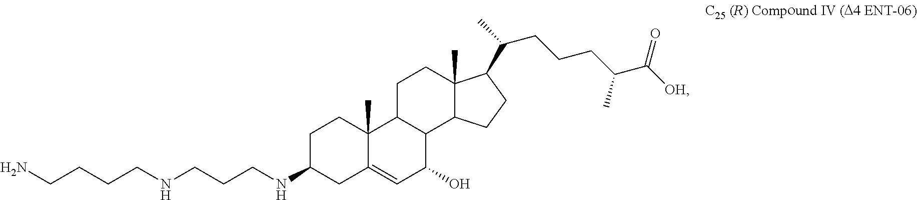 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]
or a pharmaceutically acceptable salt, solvate, prodrug, or derivative thereof; or
(b) of claim 2(c) having the formula: 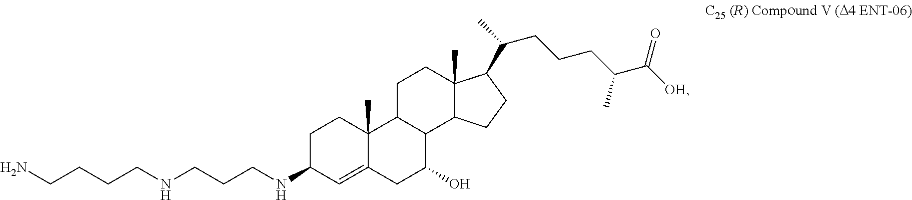 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]
or a pharmaceutically acceptable salt, solvate, prodrug, or derivative thereof; or
(c) of claim 2(d) having the formula: 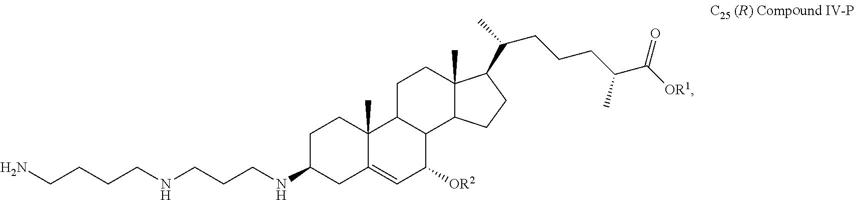
or a pharmaceutically acceptable salt, solvate, prodrug, or derivative thereof; or
(d) of claim 2(e) having the formula: 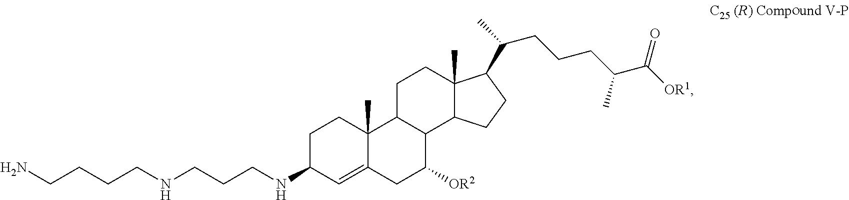
or a pharmaceutically acceptable salt, solvate, prodrug, or derivative thereof; or
(e) of claim 2(f) having the formula: 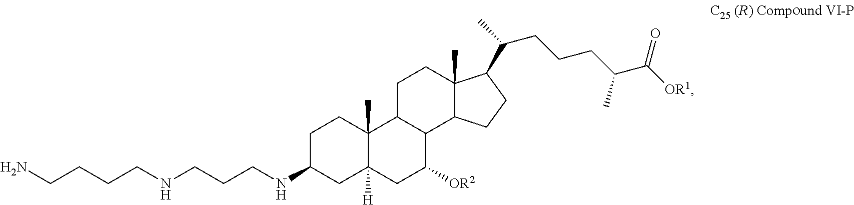
or a pharmaceutically acceptable salt, solvate, prodrug, or derivative thereof.

5. (canceled)

6. (canceled)

7. (canceled)

8. (canceled)

9. (canceled)

10. (canceled)

11. (canceled)

12. (canceled)

13. (canceled)

14. (canceled)

15. A composition comprising an aminosterol compound according to claim 1 and:
(a) at least one pharmaceutically acceptable carrier or excipient; or
(b) at least one pharmaceutically acceptable carrier or excipient selected from the group consisting of an aqueous carrier, a buffer, a sugar and a polyol compound; and/or
(c) wherein the aminosterol compound is formulated as a pharmaceutically acceptable salt; and/or
(d) wherein the aminosterol compound is formulated as a pharmaceutically acceptable salt which is a phosphate salt; and/or
(e) wherein the composition further comprises at least one additional active agent.

16. (canceled)

17. (canceled)

18. The composition of claim 15, wherein the composition is formulated:
(a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c); and/or
(e) for oral administration; and/or
(f) as an oral tablet or capsule; and/or
(g) for intranasal administration.

19. (canceled)

20. (canceled)

21. (canceled)

22. A method of:
(a) treating a subject in need having a condition susceptible to treatment with an aminosterol, comprising administering a therapeutically effective amount of the composition according to claim 15; or
(b) treating, preventing, and/or slowing the onset or progression of a condition or disorder, or a related symptom, correlated with abnormal alpha-synuclein pathology and/or dopaminergic dysfunction, in a subject in need, comprising administering a therapeutically effective amount of a composition according to claim 15; or
(c) treating, preventing, and/or slowing the onset or progression a cerebral or general ischemic disorder and/or a related symptom, correlated with abnormal alpha-synuclein pathology and/or dopaminergic dysfunction, in a subject in need, comprising administering a therapeutically effective amount of a composition according to claim 15; or
(d) inhibiting protein tyrosine phosphatase 1B (PTP1B) in a subject, the method comprising administering to the subject a therapeutically effective amount of a composition according to claim 15; or
(e) increasing gene transcription in the gut of a subject, comprising administering to the subject a therapeutically effective amount of the composition according to claim 15.

23. The method of claim 22(a), wherein the condition is correlated with abnormal alpha-synuclein pathology and/or dopaminergic dysfunction.

24. (canceled)

25. The method of claim 22(b), wherein:
(a) the symptom is selected from the group consisting of constipation, hallucinations, cognitive impairment, and inflammation;
(b) the symptom is correlated with a synucleopathy, a neurodegenerative disease, a neurological disease or disorder, a psychological and/or behavior disorder, or a cerebral or general ischemic disorder or condition; or
(c) the condition or disorder is a synucleopathy, neurodegenerative disease, or neurological disease or disorder;
(d) the condition or disorder is a psychological and/or behavior disorder; or
(e) the condition or disorder is a cerebral or general ischemic disorder or condition; or
(f) the symptom is correlated with a synucleopathy, a neurodegenerative disease, a neurological disease or disorder and wherein the synucleopathy, neurodegenerative disease, or neurological disease or disorder is selected from the group consisting of Parkinson's disease, Alzheimer's disease, schizophrenia, multiple system atrophy, Lewy body dementia, dementia with Lewy bodies, Huntington's Disease, Multiple Sclerosis, Amyotorphic Lateral Sclerosis, Friedreich's ataxia, vascular dementia, spinal muscular atrophy, supranuclear palsy, progressive nuclear palsy, fronto temperal dementia, progressive nuclear palsy, Guadeloupian Parkinsonism, spinocerebellar ataxia, parkinsonism, traumatic brain injury, degenerative processes associated with aging, and dementia of aging; or
(g) the symptom is correlated with a psychological and/or behavior disorder and wherein the psychological or behavior disorder is selected from the group consisting of depression, autism, autism spectrum disorder, down syndrome, Gaucher's disease, Krabbe's disease, lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, and sleep disorders such as REM sleep behavior disorder (RBD), sleep fragmentation, REM behavior disorder, circadian rhythm dysfunction, sleep apnea, and cognitive impairment; or
(h) the symptom is correlated with a cerebral or general ischemic disorder or condition and wherein the cerebral or general ischemic disorder or condition is selected from the group consisting of microangiopathy, intrapartum, cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure, and pulmonary edema.

26. (canceled)

27. (canceled)

28. The method of claim 22(c), wherein the cerebral or general ischemic disorder and/or a related symptom is selected from the group consisting of microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, low blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects (CCDs) and/or a related symptom, and pulmonary edema.

29. (canceled)

30. The method of claim 22, wherein:
(a) the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof; and/or
(b) the method of administration is nasal administration, oral administration, or a combination thereof; or
(c) the method of administration comprises oral administration and wherein the therapeutically effective amount of the aminosterol compound or a pharmaceutically acceptable salt, solvate, prodrug, or derivative thereof comprises:
(i) about 1 to about 300 mg/day; or
(ii) about 25 to about 500 mg/day; and/or
(d) administration of the composition comprises administration on an empty stomach, optionally within two hours of the subject waking; and/or
(e) no food is consumed by the subject after about 60 to about 90 minutes from administration of the composition; and/or
(f) the subject is a human; and/or
(g) the aminosterol compound or a pharmaceutically acceptable salt, solvate, prodrug, or derivative thereof is administered in combination with at least one additional active agent to achieve either an additive or synergistic effect.

31. (canceled)

32. The method of claim 22, wherein the therapeutically effective amount of the aminosterol compound or a pharmaceutically acceptable salt, solvate, prodrug, or derivative thereof comprises:
(a) about 0.1 to about 20 mg/kg body weight of the subject;
(b) about 0.1 to about 15 mg/kg body weight of the subject;
(c) about 0.1 to about 10 mg/kg body weight of the subject;
(d) about 0.1 to about 5 mg/kg body weight of the subject; or
(e) about 0.1 to about 2.5 mg/kg body weight of the subject.

33. The method of claim 22, wherein the therapeutically effective amount of the aminosterol compound or a pharmaceutically acceptable salt, solvate, prodrug, or derivative thereof comprises:
(a) about 0.001 to about 500 mg/day;
(b) about 0.001 to about 250 mg/day;
(c) about 0.001 to about 125 mg/day;
(d) about 0.001 to about 50 mg/day;
(e) about 0.001 to about 25 mg/day;
(f) about 0.001 to about 10 mg/day;
(g) about 0.001 to about 6 mg/day;
(h) about 0.001 to about 4 mg/day; or
(i) about 0.001 to about 2 mg/day.

34. (canceled)

35. (canceled)

36. (canceled)

37. (canceled)

38. (canceled)

39. (canceled)

40. The method of claim 22, wherein:
(a) the aminosterol, or a pharmaceutically acceptable salt, solvate, prodrug, or derivative thereof, is of pharmaceutically acceptable grade; and/or
(b) a phosphate salt of the aminosterol is administered.

41. (canceled)

42. (canceled)

43. The method of claim 22, further comprising:
(a) determining a dosage of the aminosterol or a pharmaceutically acceptable salt, solvate, prodrug, or derivative for the subject, wherein the aminosterol dosage is determined based on the effectiveness of the aminosterol dosage in improving or resolving a symptom being evaluated,
(b) followed by administering a composition comprising the dosage of the aminosterol to the subject for a period of time, wherein the method comprises:
(i) identifying a symptom to be evaluated, wherein the symptom is susceptible to treatment with an aminosterol;
(ii) identifying a starting dosage of an aminosterol thereof for the subject;
(iii) administering an escalating dosage of the aminosterol to the subject over a period of time until an effective dosage for the symptom being evaluated is identified, wherein the effective dosage is the aminosterol dosage where improvement or resolution of the symptom is observed, and fixing the aminosterol dosage at that level for that particular symptom in that particular subject.

44. The method of claim 43, wherein improvement or resolution of the symptom is measured using a clinically recognized scale or tool.

45. The method of claim 43, wherein the composition is administered orally and:
(a) the starting aminosterol dosage ranges from about 10 mg up to about 150 mg/day;
(b) the dosage of the aminosterol for the subject following escalation is fixed at a range of from about 25 mg up to about 500 mg/day; and/or
(c) the dosage of the aminosterol or a salt or derivative thereof is escalated in about 25 mg increments.

46. The method of claim 43, wherein the composition is administered intranasally and:
(a) the starting aminosterol dosage ranges from about 0.001 mg to about 3 mg/day;
(b) the dosage of the aminosterol for the subject following escalation is fixed at a range of from about 0.001 mg up to about 6 mg/day;
(c) the dosage of the aminosterol for the subject following escalation is a dosage which is subtherapeutic when given orally or by injection; and/or
(d) the dosage of the aminosterol is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

47. The method of claim 43, wherein:
(a) the dosage of the aminosterol is escalated every about 3 to about 5 days; and/or
(b) the starting aminosterol dosage is higher if the symptom being evaluated is severe; and/or
(c) the symptom is correlated with abnormal alpha-synuclein pathology and/or dopaminergic dysfunction; and/or
(d) the symptom to be evaluated is selected from the group consisting of:
(i) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue;
(ii) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing;
(iii) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor;
(iv) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia;
(v) constipation;
(vi) depression;
(vii) cognitive impairment;
(viii) sleep problems or sleep disturbances;
(ix) circadian rhythm dysfunction;
(x) hallucinations;
(xi) fatigue;
(xii) REM disturbed sleep;
(xiii) REM behavior disorder;
(xiv) erectile dysfunction;
(xv) apnea;
(xvi) postural hypotension;
(xvii) correction of blood pressure or orthostatic hypotension;
(xviii) nocturnal hypertension;
(xix) regulation of temperature;
(xx) improvement in breathing or apnea;
(xxi) correction of cardiac conduction defect;
(xxii) amelioration of pain;
(xxiii) restoration of bladder sensation and urination;
(xxiv) urinary incontinence; and/or
(xxv) control of nocturia; and/or
(e) the symptom to be evaluated is constipation, and wherein:
(i) the fixed escalated aminosterol dosage for constipation is defined as the aminosterol dosage that results in a complete spontaneous bowel movement (CSBM) within 24 hours of dosing on at least 2 of 3 days at a given dosage;
(ii) if average complete spontaneous bowel movement (CSBM) or average spontaneous bowel movement (SBM) is greater than or equal to 1 per week, then the starting aminosterol dosage prior to escalation is 75 mg/day; and/or
(iii) if average CSBM or SBM is less than 1 per week, then the starting aminosterol dosage prior to escalation is 150 mg/day.

48. (canceled)

49. (canceled)

50. (canceled)

51. (canceled)

52. (canceled)

53. The method of claim 22(e), wherein:
(a) the increase in gene transcription is for one or more genes selected from the group consisting of caspase 14, collagen type XVII alpha 1, corneodesmosin, cornifelin, cystatin E/M, dermokine, desmocollin 1, desmoglein 1 beta, filaggrin, gap junction protein beta 4, gap junction protein beta 6, H19 imprinted maternally expressed transcript, hornerin, kallikrein related-peptidase 7 chymotryptic stratum, keratin 1, keratin 10, keratinocyte differentiation associated protein, keratinocyte expressed proline-rich, late cornified envelope 1A1, late cornified envelope 1A2, late cornified envelope 1B, late cornified envelope 1C, late cornified envelope 1E, late cornified envelope 1F, late cornified envelope 1G, late cornified envelope 1H, late cornified envelope 1I, late cornified envelope 1J, late cornified envelope 1L, late cornified envelope 1M, late cornified envelope 3C, late cornified envelope 3E, late cornified envelope 3F, lectin galactose binding soluble 7, loricrin, sciellin, myoglobin, myosin binding protein C slow-type, myosin heavy polypeptide 1 skeletal muscle, myosin heavy polypeptide 8 skeletal muscle, myosin light chain phosphorylatable fast ske, myosin light polypeptide 3, myozenin 1, myozenin 2, and titin-cap; and/or
(b) the increase in gene transcription is selected from about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, about 250% to about 300%, about 300% to about 350%, about 350% to about 400%, about 400% to about 450%, about 500% to about 600%, about 600% to about 700%, about 700% to about 800%, about 800% to about 900%, about 900% to about 1000%, or about 1000% to about 1500%.

54. (canceled)

55. A method of producing an aminosterol;
(a) of formula: 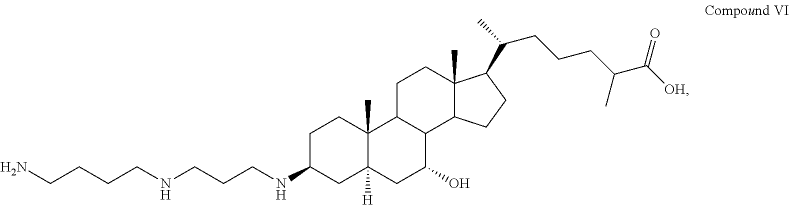 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]
comprising stimulating the addition of spermidine to Compound Ia: 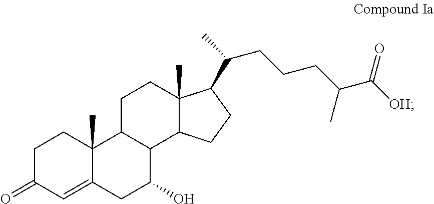 [SMILES: CC(CCC[C@@H](C)[C@H]1CCC2C3C(CC[C@@]21C)[C@@]1(C)CCC(=O)C=C1C[C@H]3O)C(=O)O]
or
(b) of formula: 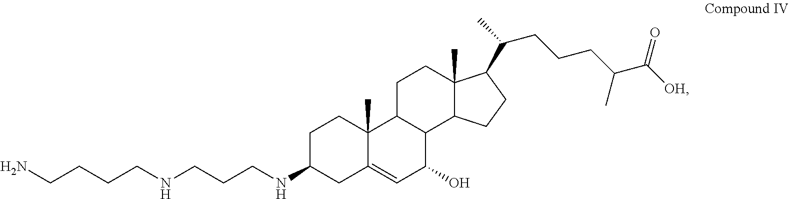 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]
comprising stimulating the addition of spermidine to Compound Ia: 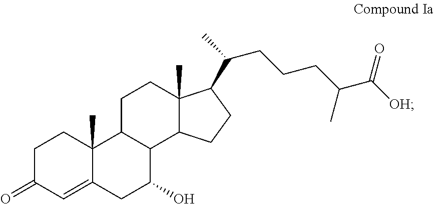 [SMILES: CC(CCC[C@@H](C)[C@H]1CCC2C3C(CC[C@@]21C)[C@@]1(C)CCC(=O)C=C1C[C@H]3O)C(=O)O]
or
(c) of formula: 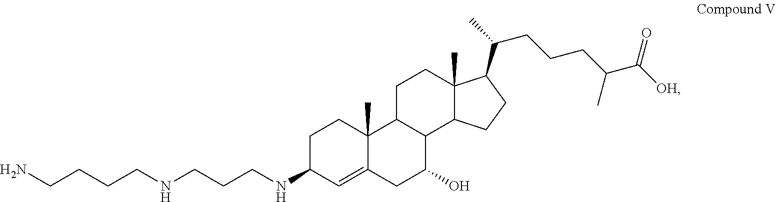 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]
comprising stimulating the addition of spermidine to Compound Ia:

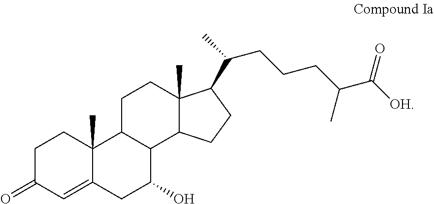 [SMILES: CC(CCC[C@@H](C)[C@H]1CCC2C3C(CC[C@@]21C)[C@@]1(C)CCC(=O)C=C1C[C@H]3O)C(=O)O]

56. The method of claim 55, wherein:
(a) the aminosterol is produced in vivo in a subject; or
(b) the aminosterol is produced in vitro.

57. A method of suppressing the formation of an aminosterol:
(a) of formula: 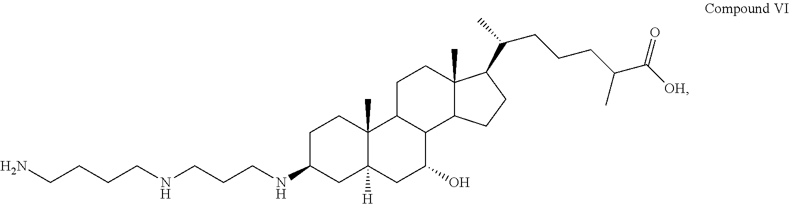 [SMILES: CC(CCC[C@@H](C)[C@H]1CCC2C3C(CC[C@@]21C)[C@@]1(C)CC[C@H](NCCCNCCCCN)C[C@@H]1C[C@H]3O)C(=O)O]
comprising suppressing the addition of spermidine to Compound Ia: 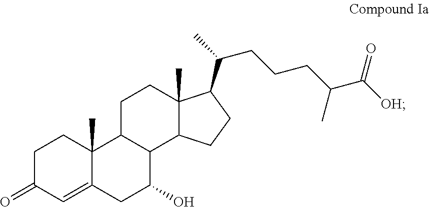 [SMILES: CC(CCC[C@@H](C)[C@H]1CCC2C3C(CC[C@@]21C)[C@@]1(C)CCC(=O)C=C1C[C@H]3O)C(=O)O]
or
(b) of formula: 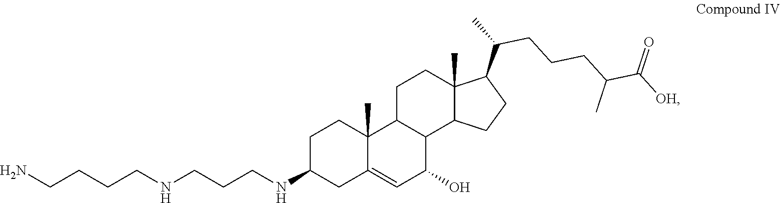 [SMILES: CC(CCC[C@@H](C)[C@H]1CCC2C3C(CC[C@@]21C)[C@@]1(C)CC[C@H](NCCCNCCCCN)CC1=C[C@H]3O)C(=O)O]
comprising suppressing the addition of spermidine to Compound Ia: 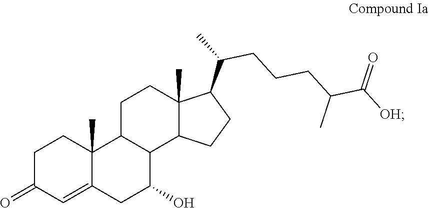 [SMILES: CC(CCC[C@@H](C)[C@H]1CCC2C3C(CC[C@@]21C)[C@@]1(C)CCC(=O)C=C1C[C@H]3O)C(=O)O]
or
(c) of formula: 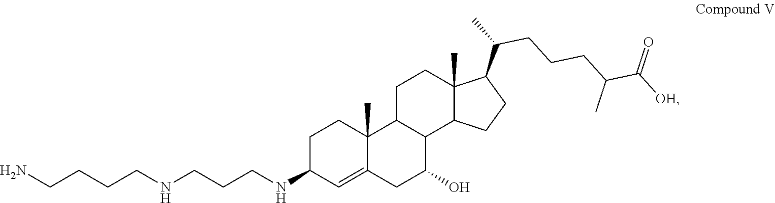 [SMILES: CC(CCC[C@@H](C)[C@H]1CCC2C3C(CC[C@@]21C)[C@@]1(C)CC[C@H](NCCCNCCCCN)C=C1C[C@H]3O)C(=O)O]
comprising suppressing the addition of spermidine to Compound Ia:

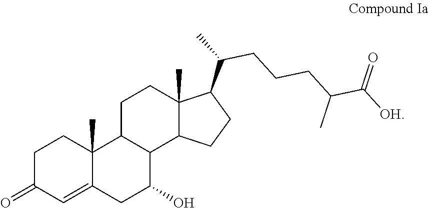 [SMILES: CC(CCC[C@@H](C)[C@H]1CCC2C3C(CC[C@@]21C)[C@@]1(C)CCC(=O)C=C1C[C@H]3O)C(=O)O]

58. method of claim 57, wherein:
(a) the addition of spermidine to Compound Ia is suppressed in vivo in a subject; or
(b) the addition of spermidine to Compound Ia is suppressed in vitro.

59. The method of claim 55(a), wherein Compound Ia has the formula: 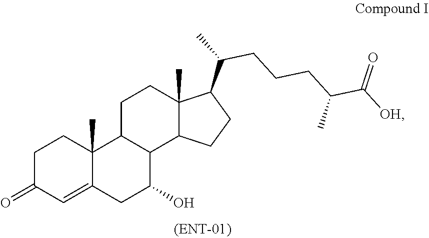 [SMILES: C[C@H](CCC[C@@H](C)[C@H]1CCC2C3C(CC[C@@]21C)[C@@]1(C)CCC(=O)C=C1C[C@H]3O)C(=O)O]
and Compound VI has the formula:

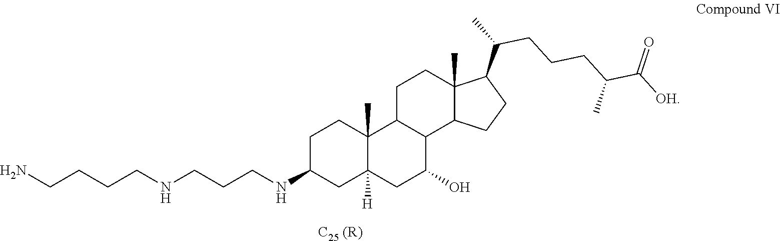 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

60. The method of claim 57(a), wherein compound Ia has the formula: 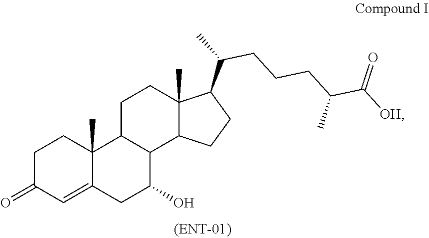 [SMILES: C[C@H](CCC[C@@H](C)[C@H]1CCC2C3C(CC[C@@]21C)[C@@]1(C)CCC(=O)C=C1C[C@H]3O)C(=O)O]
and Compound VI has the formula:

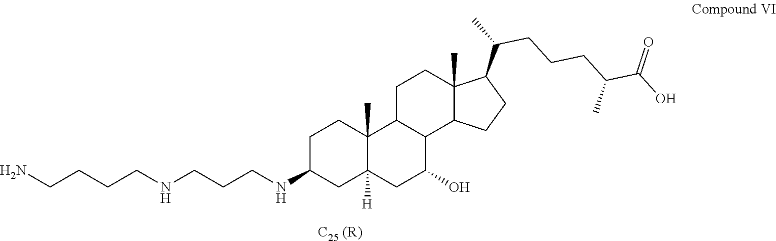 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

61. The method of claim 55(b), wherein:
Compound Ia has the formula: 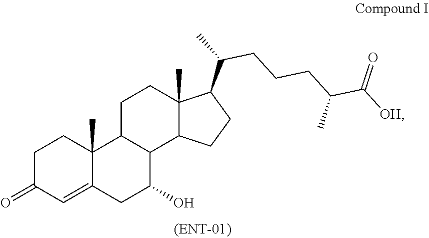 [SMILES: C[C@H](CCC[C@@H](C)[C@H]1CCC2C3C(CC[C@@]21C)[C@@]1(C)CCC(=O)C=C1C[C@H]3O)C(=O)O]
and Compound IV has the formula:

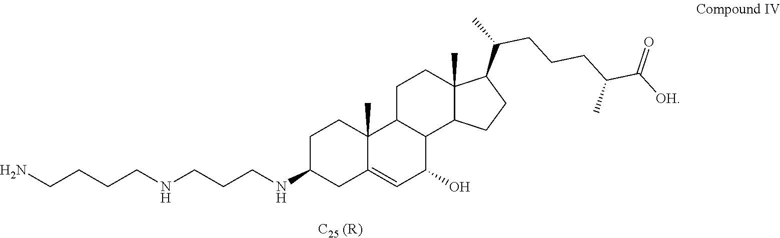 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]

62. (canceled)

63. (canceled)

64. The method of claim 57(b), wherein Compound Ia has the formula: 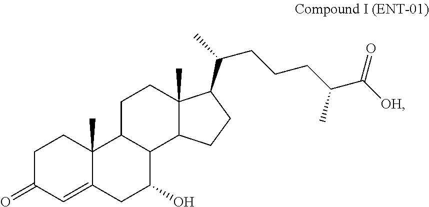 [SMILES: C[C@H](CCC[C@@H](C)[C@H]1CCC2C3C(CC[C@@]21C)[C@@]1(C)CCC(=O)C=C1C[C@H]3O)C(=O)O]
and Compound IV has the formula:

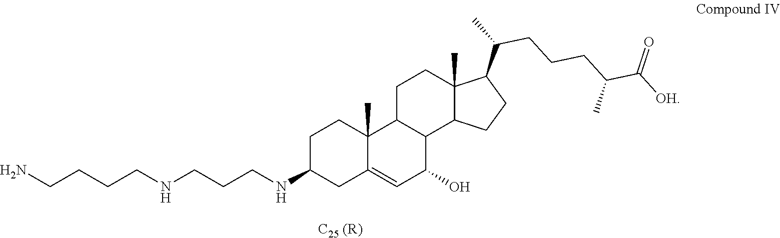 [SMILES: C[C@H](CCC[C@@H](C)[C@H]1CCC2C3C(CC[C@@]21C)[C@@]1(C)CC[C@H](NCCCNCCCCN)CC1=C[C@H]3O)C(=O)O]

65. (canceled)

66. (canceled)

67. (canceled)

68. The method of claim 55(c), wherein:
Compound Ia has the formula: 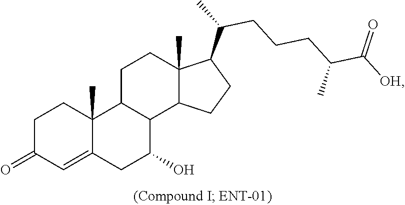 [SMILES: C[C@H](CCC[C@@H](C)[C@H]1CCC2C3C(CC[C@@]21C)[C@@]1(C)CCC(=O)C=C1C[C@H]3O)C(=O)O]
and Compound V has the formula:

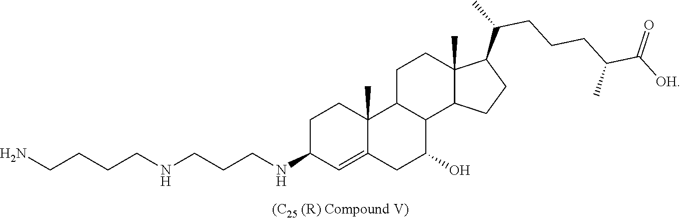 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]

69. The method of claim 57(c), wherein Compound Ia has the formula: 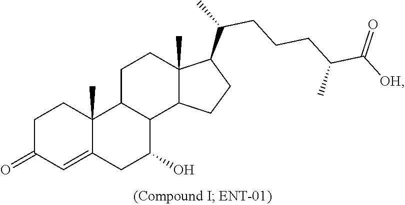 [SMILES: C[C@H](CCC[C@@H](C)[C@H]1CCC2C3C(CC[C@@]21C)[C@@]1(C)CCC(=O)C=C1C[C@H]3O)C(=O)O]
and Compound V has the formula: 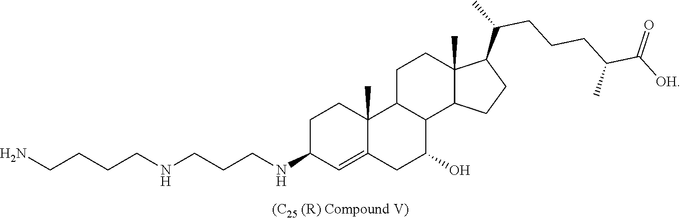 [SMILES: C[C@H](CCC[C@@H](C)[C@H]1CCC2C3C(CC[C@@]21C)[C@@]1(C)CC[C@H](NCCCNCCCCN)C=C1C[C@H]3O)C(=O)O]
